(19) United States
(12) Patent Application Publication
(10) Pub. No.: US 2002/0122768 A1
(43) Pub. Date: Sep. 5, 2002

(54) Stable radiopharmaceutical compositions and methods for preparation thereof

(76) Inventors: Shuang Liu, Chelmsford, MA (US); John A. Barrett, Groton, MA (US); Alan P. Carpenter JR., Carlisle, MA (US)

Correspondence Address: BRISTOL-MYERS SQUIBB PHARMA COMPANY, PATENT DEPARTMENT, P.O. BOX 4000, PRINCETON, NJ, 08543-4000, US

(21) Appl. No.: 09/899,629

(22) Filed: Jul. 5, 2001

Related U.S. Application Data
(60) Provisional application No. 60/216,396, filed on Jul. 6, 2000.

(51) Int. Cl. A61K 51/00

(52) U.S. Cl. 424/1.11

(57) ABSTRACT

The present invention provides stable radiopharmaceutical compositions including a therapeutic radionuclide and an effective stabilizing amount of an aromatic stabilizer (e.g., a polyhydroxylated aromatic compound, an aromatic amine, or a hydroxylated aromatic amine), alone or in combination with other antioxidants or stabilizers, to inhibit radiolytic degradation of radiopharmaceuticals. The present invention also provides improved radiopharmaceutical formulations by the use of an aromatic stabilizing agent (e.g., a polyhydroxylated aromatic compound, an aromatic amines, or a hydroxylated aromatic amine), and/or low temperature storage. The present invention also provides processes for making stable radiopharmaceutical compositions. The present invention also provides the use of the pharmaceutical compositions in medical therapy and/or medical diagnosis.

### FIELD OF THE INVENTION

This invention relates to stable radiopharmaceutical compositions comprising a therapeutic radionuclide and addition of an effective amount of an aromatic stabilizer. Particularly, this invention relates to the use of polyhydroxylated aromatic compounds, aromatic amines, and hydroxylated aromatic amines, alone or in combination with other antioxidants, as stabilizers to inhibit radiolytic degradation of radiopharmaceuticals. This invention provides improved radiopharmaceutical formulations by the use of an aromatic stabilizing agent and low temperature storage. This invention also relates to processes for making stable radiopharmaceutical compositions. The present invention is also directed to novel pharmaceutical compositions and combination therapy comprising a compound of the invention or a pharmaceutically acceptable salt thereof, and at least one agent selected from the group consisting of a chemotherapeutic agent and a radiosensitizer agent.

### BACKGROUND OF THE INVENTION

Radiopharmaceuticals are drugs that include one or more radionuclides. Radiopharmaceuticals are routinely used in nuclear medicine for the diagnosis and/or therapy of various diseases. Radiopharmaceuticals are typically small organic or inorganic compounds but can also be macromolecules, such as antibodies or antibody fragments that are not stoichiometrically labeled with a radionuclide. Radiopharmaceuticals form the chemical basis for techniques used for the diagnosis and therapy of various diseases in nuclear medicine. The in vivo diagnostic information is obtained by the intravenous injection of the radiopharmaceutical and the subsequent determination of the biodistribution using a gamma camera. The biodistribution of the radiopharmaceutical, which depends on the physical and chemical properties of the radiopharmaceutical, can be used to obtain information about the presence, progression, and/or the state of the disease.

Radiopharmaceuticals can be divided into two primary classes: those whose biodistribution is determined exclusively by their chemical and physical properties and those whose ultimate distribution is determined by their receptor binding or other biological interactions. The latter class is often called target-specific radiopharmaceuticals. In general, a target specific radiopharmaceutical can be divided into four parts: a targeting molecule, an optional linker, an optional bifunctional chelator (BFC), and a radionuclide.

The targeting molecule serves as a vehicle, which carries the radionuclide to the receptor site of the diseased tissue. The targeting molecules typically can be macromolecules, such as antibodies, or small biomolecules (BM), such as peptides, peptidomimetics, or non-peptide receptor ligands. The choice of biomolecule typically depends upon the targeted disease or the disease state.

The linker, if present, can typically be a simple hydrocarbon chain, a long poly(ethylene glycol) (PEG) chain, or a “native” poly anionic or cationic peptide sequence, which is often used for modification of pharmacokinetics. Sometimes, a metabolizeable linker is used to increase the blood clearance and to reduce the background activity, thereby improving the target-to-background ratio.

The bifunctional chelator (BFC) (i.e., metal chelator (Cₕ)) is employed to bind the metallic radionuclide to the pharmaceutical. The BFC binds strongly to the metallic radionuclide via several coordination bonds and is covalently attached to the targeting molecule either directly or through a linker. Selection of a BFC is largely determined by the nature and oxidation state of the metallic radionuclide.

The radionuclide (i.e., radioisotope) serves as the radiation source. The selection of radionuclide depends on the intended medical use (e.g., diagnostic or therapeutic) of the radiopharmaceutical. When the radionuclide is a metallic radionuclide, a BFC is typically employed to bind the metallic radionuclide to the pharmaceutical. When the radionuclide is a non-metallic radionuclide, the non-metallic radionuclide is typically linked directly, or via a linker, to the pharmaceutical.

The use of metallic radionuclides offers many opportunities for designing new radiopharmaceuticals by modifying the coordination environment around the metal with a variety of chelators. By modifying the coordination environment around the metal with a variety of chelators, the use of metallic radionuclides can offer many opportunities for designing new radiopharmaceuticals. The coordination chemistry of the metallic radionuclide will determine the geometry of the metal chelate and the solution stability of the radiopharmaceutical. Different metallic radionuclides have different coordination chemistries, and require BFCs with different donor atoms and ligand frameworks. For “metal essential” radiopharmaceuticals, the biodistribution is exclusively determined by the physical properties of the metal chelate. For target-specific radiopharmaceuticals, the “metal tag” is not totally innocent because the target uptake and biodistribution will be affected by the metal chelate, the linker, and the targeting biomolecule. This is especially true for radiopharmaceuticals based on small molecules due to the fact that in many cases the metal chelate contributes greatly to the overall size and molecular weight of the radiopharmaceutical. Therefore, the design and selection of the BFC is very important for the development of a new diagnostic or therapeutic radiopharmaceutical.

Radionuclides, such as ^{99m}Tc, ¹³¹I, ¹²³I, ^{117m}Sn, ¹¹¹In, ⁹⁷Ru, ²⁰³Pb, ⁶⁷Ga, ⁶⁸Ga, ⁸⁹Zr, and ⁶⁴Cu, have been proposed for diagnostic imaging. Nearly 80% of radiopharmaceuticals used in nuclear medicine are ^{99m}Tc-labeled compounds. The reason for such a preeminent position of ^{99m}Tc in clinical use is its extremely favorable physical and nuclear characteristics. The 6 h half-life is long enough to allow a radiochemist to carry out radiopharmaceutical synthesis and for nuclear medicine practitioners to collect useful images. At the same time, it is short enough to permit the administration of millicurie amounts of ^{99m}Tc radioactivity without significant radiation dose to the patient. The monochromatic 140 KeV photons are readily collimated to give images of superior spatial resolution. Furthermore, ^{99m}Tc is readily available from commercial ⁹⁹Mo—^{99m}Tc generators at low cost.

Radionuclides, such as ⁹⁰Y, ¹⁷⁷Lu, ¹⁴⁹Pm, ¹⁵³Sm, ¹⁶⁶Ho, ¹³¹I, ³²P, ²¹¹At, ⁴⁷Sc, ¹⁰⁹Pd, ¹⁰⁵Rh, ^{186/188}Re, and ⁶⁷Cu, are potentially useful for radiotherapy. Among these therapeutic radionuclides, lanthanide radioisotopes are of particular interest. There are several lanthanide isotopes to choose, including low energy β-emitter ¹⁷⁷Lu, medium energy β-emitters, ¹⁴⁹Pm and ¹⁵³Sm, and high-energy β-emitters, ¹⁶⁶Ho and ⁹⁰Y. Yttrium and lanthanide metals share similar coordination chemistry. The chelator technology and their coordination chemistry are well developed and well understood.

Rhenium has two isotopes (¹⁸⁶ Re and ¹⁸⁸Re) which might be useful in tumor therapy. Re has a half-life of 3.68 d with β-emission (Emax=1.07 MeV, 91% abundance) and a gamma-photon (E=137 keV, 9% abundance) which should allow imaging during therapy. ¹⁸⁸Re has a half-life of 16.98 h with an intense β-emission (Emax=2.12 MeV, 85% abundance) and 155 keV gamma photons (15% abundance). The related chemistry, medical applications, and antibody labeling with ^{186/188}Re by direct and indirect methods have recently been reviewed. Griffith, G. L., Goldenberg, D. M., Jones, A. L., Hansen, H. J.; Bioconjugate Chem. 1992, 3, 91-99 and Fritzberg, A. R., Berninger, R. W., Badley, S. W., Wester, D. W.; Pharmaceutical Res. 1988, 5, 325-334. Due to the periodic relationship, rhenium chemistry is very similar to technetium chemistry. As such, the methods used for antibody labeling with ^{99m}Tc should apply to that with ^{186/188}Re.

Identifying the most appropriate isotope for radiotherapy is often a difficult task and requires weighing a variety of factors. These include tumor uptake and retention, blood clearance, rate of radiation delivery, half-life and specific activity of the radionuclide, and the feasibility of large-scale production of the radionuclide in an economical fashion. The key point for a therapeutic radiopharmaceutical is to deliver the requisite amount of radiation dose to the tumor cells and to achieve a cytotoxic or tumoricidal effect while not causing unmanageable side-effects.

The physical half-life of the therapeutic radionuclide should match the biological half-life of the radiopharmaceutical at the tumor site. If the half-life of the radionuclide is too short, much of the decay will have occurred before the radiopharmaceutical has reached maximum target/background ratio. On the other hand, too long a half-life would cause unnecessary radiation dose to normal tissues. Ideally, the radionuclide should have a long enough half-life to attain a minimum dose rate (>0.4 Gy/h) and to irradiate all the cells during the most radiation sensitive phases of the cell cycle. In addition, the half-life of a radionuclide has to be long enough to allow adequate time for manufacturing, release, and transportation.

Other practical considerations in selecting a radionuclide for a given targeting biomolecule for tumor therapy are availability and quality. The purity has to be sufficient and reproducible, as trace amounts of impurities can affect the radiolabeling and radiochemical purity of the radiopharmaceutical. The target receptor sites in tumors are typically limited in number. This requires that the chosen radionuclide have high specific activity. The specific activity depends primarily on the production method. Trace metal contaminants must be minimized as they often compete with the radionuclide for the BFC and their metal complexes compete for receptor binding with the radiolabeled BFC-BM conjugate.

For tumor therapy, both α and β-emitters have been investigated. Alpha particles are particularly good cytotoxic agents because they dissipate a large amount of energy within one or two cell diameters. Most α-emitters are heavy elements that decay to hazardous daughter products and their penetration range is limited to only 50 μm in tissue. The short-ranged particle emitters are more attractive if the radiopharmaceutical is internalized into tumor cells. Auger electron emitters are shown to be very potent but only if they can cross the cell membrane and come into close proximity with the nucleus. This creates extra challenges for the design of new therapeutic metalloradiopharmaceuticals. The β-particle emitters have relatively long penetration range (2-12 mm in the tissue) depending the energy level. The long-range penetration is particularly important for solid tumors that have heterogeneous blood flow and/or receptor expression. The β-particle emitters yield a more homogeneous dose distribution even when they are heterogeneously distributed within the target tissue. Depending on the tumor size and location, the choice of the β-emitter may be different. For example, medium or low energy β-emitters such as ¹⁵³Sm and ¹⁷⁷Lu are better for smaller metastases while high-energy β-emitters such as ⁹⁰Y are used for larger tumors.

Therapeutic radiopharmaceutical compositions comprising β-emitting radionuclides may undergo radiolysis during preparation, release, transportation, and/or storage. During radiolysis, emissions from the radionuclide can attack other constituents of the complex or compound, or other compounds in proximity, which results in inter- and intramolecular decomposition. Radiolytic decay can result in decomposition or destruction of the radiometal chelate or the biologically active targeting molecule. Radioactivity which is not linked to the targeting biomolecule will accumulate in non-targeting tissues, and decomposition of the radiopharmaceutical composition prior to or during administration dramatically decreases the targeting potential and thus increases the toxicity of the therapeutic radiopharmaceutical composition. Thus, it is important to ensure that both the radionuclide is linked to the targeting moiety and specificity of the targeting agent is preserved.

ST receptors are found in cells of the intestine, in particular in cells in the duodenum, small intestine (jejunum and ileum), the large intestine, colon and rectum. Some metastasized intestinal cancer cells express ST receptors on their cell surfaces. Conjugated compounds which comprise an ST receptor binding moiety and a radiostable active moiety and their use in treating and radioimaging metastasized colorectal cancer are disclosed in Waldman, U.S. Pat. No. 6,060,037.

U.S. Pat. Nos. 5,093,105 and 5,306,482 disclose radiopharmaceutical compositions that include Technecium-99m radiopharmaceuticals and the radical scavenging antioxidants para-aminobenzoic acid (PABA), gentisic acid, and ascorbic acid.

U.S. Pat. No. 4,232,000 discloses Technecium-99m-based scintigraphic scanning agents that include gentisyl alcohol (2,5-dihydroxybenxyl alcohol), esters of gentisyl alcohol, salts of gentisyl alcohol, or phenolate salts of gentisyl alcohol.

U.S. Pat. No. 4,497,744 discloses Technecium-99m-based scintigraphic scanning agents that include gentisic acid (2,5-dihydroxybenzioc acid), salts of gentisic acid, or esters of gentisic acid.

U.S. Pat. No. 5,384,133 discloses gentisic acid (2,5-dihydroxybenzioc acid), salts of gentisic acid, or esters of gentisic acid alone or in combination with inositol and ascorbic acid to inhibit autoradiolysis of radiolabeled peptides.

U.S. Pat. No. 5,961,955 discloses povidone, alone or in combination with benzyl alcohol, cysteamine, cystamine, propylene glycol, dextran, and gentisic acid to be useful stabilizers for radiolabeled peptides.

U.S. Pat. No. 5,393,512 discloses therapeutic radionuclide compositions that include antioxidants such as ascorbic acid, gentisic acid, reductic acid, derivatives thereof, such as nicotinamide complexes thereof, and functionally similar compounds; challenging agents such as DTPA, EDTA, and the like; and salts, esters, amides, and mixtures thereof.

Capelle, S., et al., J. Chim. Phys., 1992, 89, 561-568, discloses the use of salicylic acid as a radical scavenging antioxidant.

Capelle, S., Redox Report, 1989, 1, 131-137, discloses the use of salicylic acid, and derivatives thereof, as a free radical (e.g., hydroxy) scavenging antioxidant.

Knapp, F., et al., Anticancer Research, 1997, 17, 1783-1796, discloses the use of ascorbic acid to stabilize radiopharmaceuticals.

PCT/US94/06276 discloses stabilizing agents such as ascorbic acid and water soluble salts and esters of ascorbic acid, gentisic acid, hydroquinone, erythrorbic acid and water soluble salts and esters of erythrorbic acid, and reductate stabilizers.

U.S. Pat. No. 6,066,309 discloses the use of ascorbic acid and derivatives thereof in stabilizing radiolabeled proteins and peptides against oxidative loss of radiolabels and autoradiolysis. Ascorbic acid is added after radiolabeling, including any required incubation period, but prior to patient administration. In addition, derivatives of ascorbic acid are defined as salts of ascorbic acid, esters of ascorbic acid, or mixtures thereof.

There continues to be a need for more effective treatment options for patients with solid tumors. This is especially true in cases of metastatic cancer in which current standard chemotherapy and external beam radiation regimens only result in marginal survival improvements.

Although improvements in cytotoxic chemotherapeutics have been made in recent years, the toxicity of these compounds to normal tissues has continued to severely limit their utility in extending survival in patients with solid tumors. Recently developed combinations of different therapeutic modalities, such as external beam irradiation and chemotherapy (i.e. chemoradiation), has provided some incremental benefit to the control of tumor progression and quality of life. However, neither systemic chemotherapeutics nor external beam irradiation have acceptable therapeutic indices, and are often limited due to unacceptable toxicity to normal tissues. The concept of combined therapy of cancer using anti-angiogenesis drugs in combination with chemotherapeutics is not new. Further, the concept of combining targeted in-vivo radiotherapy using radiolabeled antibodies and antibody fragments with chemotherapy has been reported (Stein R, Juweid M, Zhang C, et al., Clin. Cancer Res., 5: 3199s-3206s, 1999. However, the combination of a angiogenesis-targeted therapeutic radiopharmaceutical which is targeted to receptors, which are then upregulated in the neovasculature of tumors, together with chemotherapy has not been described before. Therefore, there is a need for a combination of a therapeutic radiopharmaceutical, which is targeted to localize in the neovasculature of tumors, with chemotherapeutics or a radiosensitizer agent, or a pharmaceutically acceptable salt thereof, to provide additive or synergistic therapeutic response without unacceptable additive toxicity in the treatment of solid tumors.

The major advantage of combined chemotherapy and angiogenesis-targeted therapeutic radiopharmaceuticals, over each therapeutic modality alone, is improved tumor response without substantial increases in toxicity over either treatment alone. The advantage of using neovascular-specific radiopharmaceuticals, versus a tumor-cell targeted antibody, is that there is much lower systemic radiation exposure to the subject being treated.

Further, if the receptor targets for the radiopharmaceutical compounds, used in this method of treatment, are expressed on the luminal side of tumor vessels, there is no requirement that these compounds traverse the capillary bed and bind to the tumor itself.

Thus, it is desirable to provide a combination of angiogenesis-targeted therapeutic radiopharmaceuticals and a chemotherapeutics or a radiosensitizer agent, or a pharmaceutically acceptable salt thereof, which target the luminal side of the neovasculature of tumors, to provide a surprising, and enhanced degree of tumor suppression relative to each treatment modality alone without significant additive toxicity.

There also remains a need for stable radiopharmaceutical compositions as well as stabilizing compositions for radiopharmaceuticals, particularly therapeutic radiopharmaceuticals that include radioisotope conjugates of protein, peptide, peptidomimetic or non-peptide biologically targeting groups.

### SUMMARY OF THE INVENTION

Typically, the radiopharmaceutical compositions of the present invention posses low or no toxicity when used for human administration, do not interference with the delivery or receptor binding of the radiolabeled compound to the target cells or tissue(s), and/or remain stable for a reasonable period of time (e.g., during the preparation, release, storage and/or transportation of the radiopharmaceutical composition). Typically, the stability is maintained, even when the radiopharmaceutical composition is frozen and thawed repeatedly during the period of time.

The present invention provides stable radiopharmaceutical compositions comprising a radiopharmaceutical and an effective stabilizing amount of one or more substituted aromatic compounds or substituted aromatic amines (e.g., ortho substituted hydroxy aromatic amines). The polyhydroxylated aromatic compounds and ortho-hydroxyl aromatic amines can form highly stable metal complexes with a variety of divalent and trivalent metals (e.g., Al³⁺, Ca²⁺, Fe²⁺, Fe³⁺, Cu²⁺, Ni²⁺, Zn²⁺, Pb²⁺, and La³⁺). Thus, the polyhydroxylated aromatic compounds and ortho-hydroxylated aromatic amines can act as metal scavengers to prevent trace metal contaminants from interfering in the synthesis of the radiopharmaceuticals. In addition, aromatic amines and hydroxylated aromatic amines can serve as buffers. The stabilizing agent alone or in combination with another stabilizer can provide protection against radiation induced degradation of the radiolabeled compound.

The present invention provides a pharmaceutical composition comprising:

(1.) a radiolabeled pharmaceutical agent of the formula (II):

RI-Ch-Lₙ-(BM)ₓ (II); and

an effective stabilizing amount of a compound of formula (I):

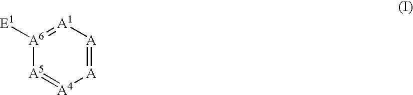

wherein

RI is ^{99m}Tc, ¹³¹I, ¹²⁵I, ¹²³I, ^{117m}Sn, ¹¹¹In, ⁹⁷Ru, ²⁰³Pb, ⁶⁷Ga, ⁶⁸Ga, ⁸⁹Zr, ⁹⁰Y, ¹⁷⁷Lu, ¹⁴⁹Pm, ¹⁵³Sm, ¹⁶⁶Ho, ¹³¹I, ³²P, ²¹¹At, ⁴⁷Sc, ¹⁰⁹Pd, ¹⁰⁵Rh, ¹⁸⁶Re, ¹⁸⁸Re, ⁶⁰Cu, ⁶²Cu, ⁶⁴Cu or ⁶⁷Cu;

Cₕ is a metal chelator or is a direct linkage (i.e., is absent);

Lₙ is a linking group or is a direct linkage (i.e., is absent);

each BM is independently a peptidomimetic or a non-peptide;

x is 1 to about 100;

E¹ is NH₂ or OH;

A¹, A², A³, A⁴ and A⁵are each independently N, C(OH) or CR¹;

provided at least one of A¹, A², A³, A⁴ and A⁵ is not CH;

each R¹ is independently —H, —C(O)R², —C(O)OR², —NHC(═O)NHR², —NHC(═S)NHR², —OC(═O)R², —OC(═O)OR², —S(O)₂OR², —C(O)NR³R⁴, —C(O)NR³OR⁴, —C(O)NR²NR³R⁴, —NR³R⁴, —NR³C(O)R⁴, —PO(OR³)(OR⁴), —S(O)₂NR³R⁴, —S(O)₂NR²NR³R⁴, —S(O)₂NR³OR⁴, C₁-C₁₀ alkyl substituted with 0-5 R⁵, C₃-C₁₀ cycloalkyl substituted with 0-5 R⁵, C₂-C₁₀ alkenyl substituted with 0-5 R⁵, or aryl substituted with 0-5 R⁵;

R², R³, and R⁴ are each independently —H, C₁-C₆ alkyl, C₃-C₆ cycloalkyl, C₁-C₆ alkenyl, benzyl, or phenyl; or R³ and R⁴ together form C₃-C₁₀ cycloalkyl or C₃-C₁₀ cycloalkenyl, optionally interrupted with —O—, —S—, —NH—, —S(═O)—, —S(O)₂—, —P(═O)(OH)—, —C(═O)NH—, —NHC(═O)—, —NHC(═O)NH—, or —NHC(═S)NH—; and

each R⁵ is independently —H, —NH₂, —OH, —CO₂H, —C(═O)NH₂, —C(═O)NHOH, —C(═O)NHNH₂, —NHC(═NH)NH₂, —NHC(═O)NH₂, —NHC(═S)NH₂, —PO₃H₂, —SO₃H, or —S(O)₂NH₂;

or a pharmaceutically acceptable salt thereof.

The pharmaceutical composition can optionally comprise an effective stabilizing amount of a second stabilizer selected from the group consisting of ascorbic acid, benzyl alcohol, gentisic acid, an ester of gentisic acid, gentisyl alcohol, an ester of gentisyl alcohol, p-aminobenzoic acid, cystamine, cystamine, 5-amino-2-hydroxybenzoic acid, nicotinic acid, nicotinamide, propylene glycol, dextran, inositol, an additional compound of formula (I), or a pharmaceutically acceptable salt thereof.

The present invention also provides a pharmaceutical composition comprising: (1.) a radiolabeled pharmaceutical agent of the formula (II):

RI-Ch-Lₙ-(BM)ₓ; (II); and

(2.) an effective stabilizing amount of a compound of formula (I):

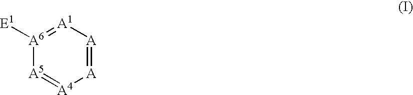

wherein

RI is ^{99m}TC, ¹³¹I, ¹²⁵I, ¹²³I, ^{117m}Sn, ¹¹¹In, ⁹⁷Ru, ²⁰³Pb, ⁶⁷Ga, ⁶⁸Ga, ⁸⁹Zr, ⁹⁰Y, ¹⁷⁷Lu, ¹⁴⁹Pm, ¹⁵³Sm, ¹⁶⁶Ho, ¹³¹I, ³²P, ²¹¹At, ⁴⁷Sc, ¹⁰⁹Pd, ¹⁰⁵Rh, ¹⁸⁶Re, ¹⁸⁸Re, ⁶⁰Cu, ⁶²Cu, ⁶⁴Cu or ⁶⁷Cu;

Cₕ is a metal chelator or is a direct linkage (i.e., absent);

Lₙ is a linking group or is a direct linkage (i.e., absent);

each BM is independently an antibody, an antibody fragment, a peptide, a peptidomimetic, or a non-peptide;

x is 1 to about 10;

E¹ is —NH₂ or —OH;

A¹, A², A³, A⁴ and A⁵ are each independently N, C(OH) or CR¹;

provided at least one of A¹, A², A³, A⁴ and A⁵ is not CH;

each R¹ is independently —H, —C(O)R², —C(O)OR², NHC(═O)NHR, —NHC(═S)NHR, —OC(═O)R, —OC(═O)OR², —S(O)₂OR, —C(O)NR³R⁴, —C(O)NR³OR⁴, —C(O)NR²NR³R⁴, —NR³R⁴, —NR³C(O)R⁴, —PO(OR³)(OR⁴), —S(O)₂NR³R⁴, —S(O)₂NR²NR³R⁴, —S(O)₂NR³OR⁴, C₁-C₁₀ alkyl substituted with 0-5 R⁵, C₃-C₁₀ cycloalkyl substituted with 0-5 R⁵, C₂-C₁₀ alkenyl substituted with 0-5 R⁵, or aryl substituted with 0-5 R⁵;

R², R³, and R⁴ are each independently H, C₁-C₆ alkyl, C₃-C₆ cycloalkyl, C₁-C₆ alkenyl, benzyl, or phenyl; or R³ and R⁴ together form C₃-C₁₀ cycloalkyl or C₃-C₁₀ cycloalkenyl, optionally interrupted with —O—, —S—, —NH—, —S(═O)—, —S(O)₂—, —P(═O)(OH)—, —C(═O)NH—, —NHC(═O)—, —NHC(═O)NH—, or —NHC(═S)NH—; and

each R⁵ is independently —H, —NH₂, —OH, —CO₂H, —C(═O)NH₂, —C(═O)NHOH, —C(═O)NHNH₂, —NHC(═NH)NH₂, —NHC(═O)NH₂, —NHC(═S)NH₂, —PO₃H₂, —SO₃H, or —S(O)₂NH₂;

or a pharmaceutically acceptable salt thereof;

provided the compound of formula (I) is not (1) a substituted monohydroxyl aromatic compound; (2) a substituted dihydroxyl aromatic compound, in which the two hydroxyl groups are not adjacent to each other; (3) a substituted monohydroxyl-monoamino aromatic compound, in which the hydroxyl group and amino group are not adjacent to each other; or (4) an ortho, meta, or para aminobenzioc acid.

The pharmaceutical composition can optionally comprise an effective stabilizing amount of a second stabilizer selected from the group consisting of ascorbic acid, benzyl alcohol, gentisic acid, an ester of gentisic acid, gentisyl alcohol, an ester of gentisyl alcohol, p-aminobenzoic acid, cystamine, cystamine, 5-amino-2-hydroxybenzoic acid, nicotinic acid, nicotinamide, propylene glycol, dextran, inositol, an additional compound of formula (I), or a pharmaceutically acceptable salt thereof.

The present invention also provides a method for preparing a stable radiopharmaceutical composition of the present invention. The method comprises combining in the absence of oxygen, the radiolabeled pharmaceutical agent of the formula RI-Ch-Lₙ-(BM)ₓ and an effective stabilizing amount of the stabilizer of the formula (I).

The present invention also provides a method for preparing a stable radiopharmaceutical composition of the present invention. The method comprises

a) combining in a container, in the absence of oxygen, the radiolabeled pharmaceutical agent of the formula RI-Ch-Lₙ-(BM)ₓ and an effective stabilizing amount of the stabilizer of the formula (I);

b) maintaining an oxygen free head-space in the container;

c) cooling the container to a temperature of less than about −20° C.; and

d) storing the container to a temperature of less than about −20° C.

The present invention also provides a method for treating or preventing thromboembolic disorders, atherosclerosis, infection, inflammation, transplant rejection, and/or cancer in an animal (e.g., mammalian) tissue inflicted with or at risk thereof comprising contacting the mammalian tissue with an effective amount of a composition of the present invention.

The present invention also provides a method for treating or preventing a disease state that is associated with the following receptors: a cyclic IIb/IIIa receptor, a fibrinogen receptor, a myocardial receptor, a renal receptor, LTβ4, selectin, growth factor (PDGF, VEGF, EGF, FGF, TNF, MCSF or an interleukin Il1-8), a receptor that is expressed or upregulated in angiogenic tumor vasculature, αvβ3, αvβ5, α5β1, α4β1, α1β1, or α2β2, α5β1, αvβ3, α5β1, or tyrosine kinases (e.g., epidermal growth factor receptor (EGFR) family in a mammalian tissue inflicted with or at risk thereof. The method comprises contacting the mammalian tissue with an effective amount of a composition of the present invention.

The present invention also provides a method for treating or preventing thromboembolic disorders, atherosclerosis, infection, inflammation, transplant rejection, and/or cancer in a mammal inflicted with or at risk thereof comprising administering to the mammal in need of such treatment or prevention an effective amount of a composition of the present invention.

The present invention also provides a method for treating or preventing a disease state that is associated with the following receptors: a cyclic IIb/IIIa receptor, a fibrinogen receptor, a myocardial receptor, a renal receptor, LTβ4, selectin, growth factor (PDGF, VEGF, EGF, FGF, TNF MCSF or an interleukin Il1-8), a receptor that is expressed or upregulated in angiogenic tumor vasculature, αvβ3, αvβ5, α5β1, α4β1, α1β1, or α2β2, α5β1, αvβ3, α5β1, or tyrosine kinases (e.g., epidermal growth factor receptor (EGFR) family in a mammal inflicted with or at risk thereof comprising administering to the mammal in need of such treatment or prevention an effective amount of a composition of the present invention.

The present invention also provides a method for imaging a tumor on or in a mammalian tissue inflicted with a tumor comprising contacting the mammalian tissue with an effective amount of a composition of the present invention; and detecting the presence of the radiolabeled pharmaceutical.

The present invention also provides a method for imaging a tumor in a mammal inflicted with a tumor comprising administering to the mammal an effective amount of a composition of the present invention; and detecting the presence of the radiolabeled pharmaceutical.

The present invention also provides a pharmaceutical composition for use in medical therapy or diagnosis.

The present invention also provides a pharmaceutical composition for the manufacture of a medicament for imaging and/or treating a tumor in a mammal.

The present invention also provides a diagnostic composition comprising an effective diagnostic amount of a radiolabeled agent RI-Ch-Ln-(BM)ₓ, an effective stabilizing amount of a compound of formula (I), and a physiologically acceptable carrier or excipient.

The present invention also provides a compound of formula (I) for use in preparing a stable radio-imaging composition.

The present invention also provides a scintigraphic diagnostic composition comprising an effective stabilizing amount of a compound of formula (I) and a radiolabeled agent RI-Ch-Ln-(BM)ₓ.

The present invention also provides a method of in vivo radio-imaging. The method comprises introducing a radioisotope (RI) to a solution comprising a compound Ch-Ln-(BM)ₓ and an effective stabilizing amount of a compound of formula (I) to form a labeled solution, administering the labeled solution in vivo; and detecting localization of the radioisotope in vivo.

The present invention also provides a method of in vitro radio-imaging a targeted receptor of a tissue. The method comprises administering an effective diagnostic amount of a radio-labeled composition of the present invention to the tissue and detecting localization of the radiolabeled agent at the targeted receptor.

The present invention also provides a method of radio-imaging a targeted site within a patient's body. The method comprises administering an effective diagnostic amount of a radio-labeled composition of the present invention; and detecting localization of the radiolabeled agent at the targeted site.

The present invention also provides a method of radio-imaging for prostate cancer or other tissues having an androgen receptor in a patient. The method comprises administering an effective diagnostic amount of a composition of the present invention; and

detecting the presence of the radiolabeled agent RI-Ch-Ln-(BM)ₓ bound to the androgen receptor.

The present invention also provides a method of radio-imaging metastasized cancer cells. The method comprises contacting an effective diagnostic amount of a radiolabeled agent RI-Ch-Ln-(BM)ₓ of the present invention and an effective stabilizing amount of a compound of formula (I) of the present invention, with a composition comprising ST receptor wherein the radiolabeled agent is capable of targeting a ST receptor.

The present invention also provides a method of radio-imaging a patient's organ. The method comprises administering an effective diagnostic amount of a radiolabeled agent RI-Ch-Ln-(BM)ₓ of the present invention, and an effective stabilizing amount of a compound of formula (I) of the present invention to a patient in need of such radioimaging; and detecting the presence of the radiolabeled agent bound to the organ.

The present invention also provides a method of delivering a radionuclide to a target location. The method comprises providing a radiolabeled agent RI-Ch-Ln-(BM)ₓ of the present invention and providing an effective stabilizing amount of a compound of formula (I) of the present invention.

The present invention also provides a kit for preparing a radio-imaging composition. The kit comprises a sealed vial containing a predetermined quantity of a radiolabeled agent RI-Ch-Ln-(BM)ₓ of the present invention and an effective stabilizing amount of a compound of formula (I) of the present invention.

The present invention also provides a kit comprising a plurality-vial system of a radio-imaging composition of the invention and a diluent. The kit comprises (a) a first vial comprising a predetermined quantity of a radiolabelled agent RI-Ch-Ln-(BM)ₓ of the present invention and an effective stabilizing amount of a compound of formula (I) of the present invention; and (b) a second vial comprising a pharmaceutically acceptable carrier or diluent.

The present invention also provides a pharmaceutical composition comprising a radiolabeled agent RI-Ch-Ln-(BM)ₓ of the present invention, an effective stabilizing amount of a compound of formula (I) of the present invention, and optionally an effective stabilizing amount of a second stabilizer selected from the group consisting of ascorbic acid, benzyl alcohol, gentisic acid, an ester of gentisic acid, gentisyl alcohol, an ester of gentisyl alcohol, p-aminobenzoic acid, cystamine, cystamine, 5-amino-2-hydroxybenzoic acid, nicotinic acid, nicotinamide, propylene glycol, dextran, inositol, a compound of formula (I), or a pharmaceutically acceptable salt thereof, and a pharmaceutically acceptable carrier.

The present invention also provides a method of preparing a stable radiopharmaceutical composition. The method comprises providing a radiolabeled agent RI-Ch-Ln-(BM)ₓ of the present invention and providing an effective stabilizing amount of a compound of formula (I) of the present invention to provide a stable radiopharmaceutical composition.

The present invention also provides a method of treating cancer. The method comprises administering to a patient, in need thereof, a therapeutically effective amount of a pharmaceutical composition of the present invention and optionally at least one agent selected from the group consisting of a chemotherapeutic agent and a radiosensitizer agent, or a pharmaceutically acceptable salt thereof.

The present invention also provides a kit for treating cancer. The kit comprises a therapeutically effective amount of a pharmaceutical composition of the present invention and optionally at least one agent selected from the group consisting of a chemotherapeutic agent and a radiosensitizer agent, or a pharmaceutically acceptable salt thereof.

### DETAILED DESCRIPTION OF THE INVENTION

The compounds described herein may have one or more asymmetric centers. Compounds described herein having one or more asymmetrically substituted atoms may be isolated in optically active or racemic forms. It is well known in the art how to prepare optically active forms, such as by resolution of racemic forms or by synthesis from optically active starting materials. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. All chiral, diastereomeric, racemic forms and all geometric isomeric forms of a structure are intended, unless the specific stereochemistry or isomeric form is specifically indicated. All processes used to prepare compounds described herein and intermediates made herein are considered to be part of the present invention.

The present invention is intended to include all isotopes of atoms occurring in the present compounds. Isotopes include those atoms having the same atomic number but different mass numbers. By way of general example and without limitation, isotopes of hydrogen include tritium and deuterium. Isotopes of carbon include Carbon-13 and Carbon-14.

The term “substituted,” as used herein, means that any one or more hydrogens on the designated atom is replaced with a selection from the indicated group, provided that the designated atom's normal valency is not exceeded, and that the substitution results in a stable compound. When a substituent is keto (i.e.,═O), then two hydrogens on the atom are replaced. Keto substituents are not present on aromatic moieties. When a ring system (e.g., carbocyclic or heterocyclic) is said to be substituted with a carbonyl group or a double bond, it is intended that the carbonyl group or double bond be part (i.e., within) of the ring.

When any variable (e.g., R⁵) occurs more than one time in any constituent or formula for a compound, its definition at each occurrence is independent of its definition at every other occurrence. Thus, for example, if a group is shown to be substituted with 0-2 R⁵, then the group may optionally be substituted with up to two R⁵ groups, and R⁵ at each occurrence is selected independently from the definition of the other R⁵. Also, combinations of substituents and/or variables are permissible only if such combinations result in stable compounds.

When a bond to a substituent is shown to cross a bond connecting two atoms in a ring, then such substituent may be bonded to any suitable atom on the ring. When a substituent is listed without indicating the atom via which such substituent is bonded to the rest of the compound of a given formula, then such substituent may be bonded via any atom in such substituent. Combinations of substituents and/or variables are permissible only if such combinations result in stable compounds.

As used herein, “alkyl” is intended to include both branched and straight-chain saturated aliphatic hydrocarbon groups having the specified number of carbon atoms. Examples of alkyl include, but are not limited to, methyl, ethyl, n-propyl, i-propyl, n-butyl, s-butyl, t-butyl, n-pentyl, and s-pentyl.

As used herein, “haloalkyl” is intended to include both branched and straight-chain saturated aliphatic hydrocarbon groups having the specified number of carbon atoms, substituted with 1 or more halogen (for example —CᵥF_{w} where v=1 to 3 and w=1 to (2v+1)). Examples of haloalkyl include, but are not limited to, trifluoromethyl, trichloromethyl, pentafluoroethyl, and pentachloroethyl.

As used herein, “alkoxy” represents an alkyl group as defined above with the indicated number of carbon atoms attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy.

As used herein, “cycloalkyl” is intended to include saturated ring groups, such as cyclopropyl, cyclobutyl, or cyclopentyl.

As used herein, “alkenyl” is intended to include hydrocarbon chains of either a straight or branched configuration having one or more unsaturated carbon-carbon bonds, which may occur in any stable point along the chain, such as ethenyl and propenyl.

As used herein, “alkynyl” is intended to include hydrocarbon chains of either a straight or branched configuration having one or more triple carbon-carbon bonds, which may occur in any stable point along the chain, such as ethynyl and propynyl. “Halo” or “halogen” as used herein refers to fluoro, chloro, bromo, and iodo; and “counterion” is used to represent a small, negatively charged species such as chloride, bromide, hydroxide, acetate, and sulfate.

As used herein, “carbocycle” or “carbocyclic residue” is intended to mean any stable 3- to 7-membered monocyclic or bicyclic, or 7- to 13-membered bicyclic or tricyclic ring system, any of which may be saturated (i.e. a cycloalkyl group), partially unsaturated (i.e. a cycloalkenyl group), or aromatic (i.e. an aryl group). Examples of such carbocycles include, but are not limited to, cyclopropyl, cyclobutyl, cyclopentyl, cyclohexyl, cycloheptyl, adamantyl, cyclooctyl, [3.3.0]bicyclooctane, [4.3.0]bicyclononane, [4.4.0]bicyclodecane, [2.2.2]bicyclooctane, fluorenyl, phenyl, naphthyl, indanyl, adamantyl, and tetrahydronaphthyl.

As used herein, “aryl” means aromatic carbocyclic radical containing about 6 to about 10 carbon atoms. Exemplary aryl include phenyl or naphthyl.

As used herein, “cycloalkyl” means a non-aromatic mono- or multicyclic ring system of about 3 to about 10 carbon atoms. Preferred monocyclic cycloalkyl rings include cyclopentyl, cyclohexyl, and cycloheptyl. Exemplary multicyclic cycloalkyl rings include 1-decalin, adamant-(1- or 2-)yl, [2.2.1]bicycloheptanyl (norbornyl).

As used herein, “cycloalkenyl” means a non-aromatic monocyclic or multicyclic ring system containing a carbon-carbon double bond and having about 3 to about 10 carbon atoms. Preferred unsubstituted or substituted monocyclic cycloalkenyl rings include cyclopentenyl, cyclohexenyl, and cycloheptenyl. Preferred multicyclic cycloalkenyl rings include [2.2.1 ]bicycloheptenyl (norbornenyl) and [2.2.2]bicyclooctenyl.

As used herein, the term “heterocycle” or “heterocyclic system” is intended to mean a saturated heterocycle group (i.e. a heterocyclyl group), a partially unsaturated heterocycle group (i.e. a heterocyclenyl group), or a unsaturated heterocycle group (i.e. a heteroaryl group) as described herein, which consists of carbon atoms and from 1 to 4 heteroatoms independently selected from the group consisting of N, O and S and including any bicyclic group in which any of the above-defined heterocyclic rings is fused to a benzene ring. The nitrogen and sulfur heteroatoms may optionally be oxidized. The heterocyclic ring may be attached to its pendant group at any heteroatom or carbon atom which results in a stable structure. The heterocyclic rings described herein may be substituted on carbon or on a nitrogen atom if the resulting compound is stable. If specifically noted, a nitrogen in the heterocycle may optionally be quaternized. It is preferred that when the total number of S and O atoms in the heterocycle exceeds 1, then these heteroatoms are not adjacent to one another. It is preferred that the total number of S and O atoms in the heterocycle is not more than 1.

Examples of heterocycles include, but are not limited to, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

Preferred heterocycles include, but are not limited to, pyridinyl, furanyl, thienyl, pyrrolyl, pyrazolyl, pyrrolidinyl, imidazolyl, indolyl, benzimidazolyl, 1H-indazolyl, oxazolidinyl, benzotriazolyl, benzisoxazolyl, oxindolyl, benzoxazolinyl, and isatinoyl. Also included are fused ring and spiro compounds containing, for example, the above heterocycles.

As used herein, “heterocyclenyl” means a non-aromatic monocyclic or multicyclic hydrocarbon ring system of about 3 to about 10 atoms, preferably about 4 to about 8 atoms, in which one or more of the carbon atoms in the ring system is/are hetero element(s) other than carbon, for example nitrogen, oxygen or sulfur atoms, and which contains at least one carbon-carbon double bond or carbon-nitrogen double bond. Preferred ring sizes of rings of the ring system include about 5 to about 6 ring atoms. The designation of the aza, oxa or thia as a prefix before heterocyclenyl define that at least a nitrogen, oxygen or sulfur atom is present respectively as a ring atom. The nitrogen or sulphur atom of the heterocyclenyl may also be optionally oxidized to the corresponding N-oxide, S-oxide or S,S-dioxide. “Heterocyclenyl” as used herein includes by way of example and not limitation those described in Paquette, Leo A.; “Principles of Modem Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and “J. Am. Chem. Soc.”, 82:5566 (1960). Exemplary monocyclic azaheterocyclenyl groups include 1,2,3,4-tetrahydrohydropyridine, 1,2-dihydropyridyl, 1,4-dihydropyridyl, 1,2,3,6-tetrahydropyridine, 1,4,5,6-tetrahydropyrimidine, 2-pyrrolinyl, 3-pyrrolinyl, 2-imidazolinyl, 2-pyrazolinyl, and the like. Exemplary oxaheterocyclenyl groups include 3,4-dihydro-2H-pyran, dihydrofuranyl, and fluorodihydrofuranyl. Preferred is dihydrofuranyl. An exemplary multicyclic oxaheterocyclenyl group is 7-oxabicyclo[2.2.1]heptenyl. Preferred monocyclic thiaheterocycleny rings include dihydrothiophenyl and dihydrothiopyranyl; more preferred is dihydrothiophenyl.

As used herein, “heterocyclyl” means a non-aromatic saturated monocyclic or multicyclic ring system of about 3 to about 10 carbon atoms, preferably about 4 to about 8 carbon atoms, in which one or more of the carbon atoms in the ring system is/are hetero element(s) other than carbon, for example nitrogen, oxygen or sulfur. Preferred ring sizes of rings of the ring system include about 5 to about 6 ring atoms. The designation of the aza, oxa or thia as a prefix before heterocyclyl define that at least a nitrogen, oxygen or sulfur atom is present respectively as a ring atom. The nitrogen or sulphur atom of the heterocyclyl may also be optionally oxidized to the corresponding N-oxide, S-oxide or S,S-dioxide. “Heterocyclyl” as used herein includes by way of example and not limitation those described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and “J. Am. Chem. Soc.”, 82:5566 (1960). Exemplary monocyclic heterocyclyl rings include piperidyl, pyrrolidinyl, piperazinyl, morpholinyl, thiomorpholinyl, thiazolidinyl, 1,3-dioxolanyl, 1,4-dioxanyl, tetrahydrofuranyl, tetrahydrothiophenyl, tetrahydrothiopyranyl, and the like.

As used herein, “heteroaryl” or “aromatic heterocyclic system” means an aromatic monocyclic or multicyclic ring system of about 5 to about 10 atoms, in which one or more of the atoms in the ring system is/are hetero element(s) other than carbon, for example nitrogen, oxygen or sulfur. Preferred ring sizes of rings of the ring system include about 5 to about 6 ring atoms. The designation of the aza, oxa or thia as a prefix before heteroaryl define that at least a nitrogen, oxygen or sulfur atom is present respectively as a ring atom. A nitrogen atom of an heteroaryl may be optionally oxidized to the corresponding N-oxide. Heteroaryl as used herein includes by way of example and not limitation those described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and “J. Am. Chem. Soc.”, 82:5566 (1960). Exemplary heteroaryl and substituted heteroaryl groups include pyrazinyl, thienyl, isothiazolyl, oxazolyl, pyrazolyl, furazanyl, pyrrolyl, 1,2,4-thiadiazolyl, pyridazinyl, quinoxalinyl, phthalazinyl, imidazo[1,2-a]pyridine, imidazo[2,1-b]thiazolyl, benzofurazanyl, azaindolyl, benzimidazolyl, benzothienyl, thienopyridyl, thienopyrimidyl, pyrrolopyridyl, imidazopyridyl, benzoazaindole, 1,2,4-triazinyl, benzthiazolyl, furanyl, imidazolyl, indolyl, indolizinyl, isoxazolyl, isoquinolinyl, isothiazolyl, oxadiazolyl, pyrazinyl, pyridazinyl, pyrazolyl, pyridyl, pyrimidinyl, pyrrolyl, quinazolinyl, quinolinyl, 1,3,4-thiadiazolyl, thiazolyl, thienyl and triazolyl. Preferred heteroaryl groups include pyrazinyl, thienyl, pyridyl, pyrimidinyl, isoxazolyl and isothiazolyl.

The term “amino acid” as used herein means an organic compound containing both a basic amino group and an acidic carboxyl group. Included within this term are natural amino acids (e.g., L-amino acids), modified and unusual amino acids (e.g., D-amino acids), as well as amino acids which are known to occur biologically in free or combined form but usually do not occur in proteins. Included within this term are modified and unusual amino acids, such as those disclosed in, for example, Roberts and Vellaccio, The Peptides, 1983, 5, 342-429.

Natural protein occurring amino acids include, but are not limited to, alanine, arginine, asparagine, aspartic acid, cysteine, glutamic acid, glutamine, glycine, histidine, isoleucine, leucine, lysine, methionine, phenylalanine, serine, threonine, tyrosine, tyrosine, tryptophan, proline, and valine. Natural non-protein amino acids include, but are not limited to arginosuccinic acid, citrulline, cysteine sulfinic acid, 3,4-dihydroxyphenylalanine, homocysteine, homoserine, omithine, 3-monoiodotyrosine, 3,5-diiodotryosine, 3,5,5′-triiodothyronine, and 3,3′,5,5′-tetraiodothyronine. Modified or unusual amino acids which can be used to practice the invention include, but are not limited to, D-amino acids, hydroxylysine, 4-hydroxyproline, an N-Cbz-protected amino acid, 2,4-diaminobutyric acid, homoarginine, norleucine, N-methylaminobutyric acid, naphthylalanine, phenylglycine, B-phenylproline, tert-leucine, 4-aminocyclohexylalanine, N-methyl-norleucine, 3,4-dehydroproline, N,N-dimethylaminoglycine, N-methylaminoglycine, 4-aminopiperidine-4-carboxylic acid, 6-aminocaproic acid, trans-4-(aminomethyl)cyclohexanecarboxylic acid, 2-, 3-, and 4-(aminomethyl)benzoic acid, 1-aminocyclopentanecarboxylic acid, 1-aminocyclopropanecarboxylic acid, and 2-benzyl-5-aminopentanoic acid.

The term “peptide” as used herein means a linear compound having two or more amino acids (as defined herein) that are linked by means of a peptide bond. A “peptide” as used in the presently claimed invention is intended to refer to a moiety with a molecular weight of less than 10,000 Daltons, preferable less than 5,000 Daltons, and more preferably less than 2,500 Daltons. The term “peptide” also includes compounds containing both peptide and non-peptide components, such as pseudopeptide, peptidomimetic residues, or other non-amino acid components. Such a compound containing both peptide and non-peptide components may also be referred to as a “peptide analog”.

A “pseudopeptide” or “peptidomimetic” is a compound which mimics the structure of an amino acid residue or a peptide, for example, by using linking groups other than amide linkages between the peptide mimetic and an amino acid residue (pseudopeptide bonds) and/or by using non-amino acid substituents and/or a modified amino acid residue. A “pseudopeptide residue” means that portion of an pseudopeptide or peptidomimetic that is present in a peptide.

The term “peptide bond” means a covalent amide linkage formed by loss of a molecule of water between the carboxyl group of one amino acid and the amino group of a second amino acid.

The term “pseudopeptide bonds” includes peptide bond isosteres which may be used in place of or as substitutes for the normal amide linkage. These substitute or amide “equivalent” linkages are formed from combinations of atoms not normally found in peptides or proteins which mimic the spatial requirements of the amide bond and which should stabilize the molecule to enzymatic degradation.

The term “non-peptide” refers to a compound having preferably less than three amide bonds in the backbone core compound or preferably less than three amino acids or amino acid mimetics.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making an acid or base salt thereof. Examples of pharmaceutically acceptable salts include, but are not limited to, mineral or organic acid salts of basic residues such as amines; and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic.

The pharmaceutically acceptable salts of the present invention can be synthesized from the parent compound having a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 1985, 17th ed., Mack Publishing Company, Easton, Pa., p. 1418.

“Stable compound” and “stable structure” are meant to indicate a compound that is sufficiently robust to survive isolation to a useful degree of purity from a reaction mixture, and formulation into an efficacious therapeutic agent.

“Stable radiopharmaceutical composition” refers to a radiopharmaceutical composition that includes one or more stabilizers, effective to inhibit, slow down, or diminish the degradation of radiopharmaceuticals that would occur in the absence of the one or more stabilizers.

The phrase “pharmaceutically acceptable prodrugs” as used herein means those prodrugs of the compounds useful according to the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the invention.

The term “prodrug” means compounds that are rapidly transformed in vivo to yield the parent compound of the above formula, for example by hydrolysis in blood. Functional groups which may be rapidly transformed, by metabolic cleavage, in vivo form a class of groups reactive with the carboxyl group of the compounds of this invention. They include, but are not limited to such groups as alkanoyl (such as acetyl, propionyl, butyryl, and the like), unsubstituted and substituted aroyl (such as benzoyl and substituted benzoyl), alkoxycarbonyl (such as ethoxycarbonyl), trialkylsilyl (such as trimethyl- and triethysilyl), monoesters formed with dicarboxylic acids (such as succinyl), and the like. Because of the ease with which the metabolically cleavable groups of the compounds useful according to this invention are cleaved in vivo, the compounds bearing such groups act as pro-drugs. The compounds bearing the metabolically cleavable groups have the advantage that they may exhibit improved bioavailability as a result of enhanced solubility and/or rate of absorption conferred upon the parent compound by virtue of the presence of the metabolically cleavable group.

A thorough discussion of prodrugs is provided in the following: Design of Prodrugs, H. Bundgaard, ed., Elsevier, 1985; Methods in Enzymology, K. Widder et al, Ed., Academic Press, 42, p.309-396, 1985; A Textbook of Drug Design and Development, Krogsgaard-Larsen and H. Bundgaard, ed., Chapter 5; “Design and Applications of Prodrugs” p.113-191, 1991; Advanced Drug Delivery Reviews, H. Bundgard, 8, p.1-38, 1992; Journal of Pharmaceutical Sciences, 77, p. 285, 1988; Chem. Pharm. Bull., N. Nakeya et al, 32, p. 692, 1984; Pro-drugs as Novel Delivery Systems, T. Higuchi and V. Stella, Vol. 14 of the A.C.S. Symposium Series, and Bioreversible Carriers in Drug Design, Edward B. Roche, ed., American Pharmaceutical Association and Pergamon Press, 1987.

Lyophilization aids useful in the preparation of diagnostic kits useful for the preparation of radiopharmaceuticals include but are not limited to mannitol, lactose, sorbitol, dextran, Ficoll, and polyvinylpyrrolidine (PVP).

Solubilization aids useful in the preparation of radiopharmaceuticals and in diagnostic kits useful for the preparation of said radiopharmaceuticals include but are not limited to ethanol, glycerin, polyethylene glycol, propylene glycol, polyoxyethylene sorbitan monooleate, sorbitan monoloeate, polysorbates, poly(oxyethylene)poly(oxypropylene)poly(oxyethylene) block copolymers (Pluronics) and lecithin. Preferred solubilizing aids are polyethylene glycol, and Pluronics.

Bacteriostats useful in the preparation of radiopharmaceuticals and in diagnostic kits useful for the preparation of said radiopharmaceuticals include but are not limited to benzyl alcohol, benzalkonium chloride, chlorbutanol, and methyl, propyl or butyl paraben.

Preferably, the compound of formula (I) is not include gentisic acid (2,5-dihydroxybenzioc acid), salts of gentisic acid, or esters of gentisic acid, as disclosed in U.S. Pat. Nos. 4,497,744 or 5,384,133. Esters of gentisic acid include those compounds wherein gentisic acid is esterified at the carboxyl moiety and/or the one or two hydroxyl groups, as disclosed in U.S. Pat. Nos. 4,497,744 and 5,384,133.

Furthermore, the compound of formula (I) is preferably not gentisyl alcohol (2,5-dihydroxybenxyl alcohol), esters of gentisyl alcohol, salts of gentisyl alcohol, or phenolate salts of gentisyl alcohol. Esters of gentisyl alcohol include those compounds wherein gentisyl alcohol is esterified at the primary hydroxyl group (i.e., CH₂OH) and/or the one or two secondary hydroxyl groups, as disclosed in U.S. Pat. No. 4,232,000.

Administration of a compound of the present invention in combination with such additional therapeutic agents, may afford an efficacy advantage over the compounds and agents alone, and may do so while permitting the use of lower doses of each. A lower dosage minimizes the potential of side effects, thereby providing an increased margin of safety. The combination of a compound of the present invention with such additional therapeutic agents is preferably a synergistic combination. Synergy, as described for example by Chou and Talalay, Adv. Enzvme Regul. 22:27-55 (1984), occurs when the therapeutic effect of the compound and agent when administered in combination is greater than the additive effect of the either the compound or agent when administered alone. In general, a synergistic effect is most clearly demonstrated at levels that are (therapeutically) sub-optimal for either the compound of the present invention, a chemotherapeutic agent or a radiosensitizer agent alone, but which are highly efficacious in combination. Synergy can be in terms of improved tumor response without substantial increases in toxicity over individual treatments alone, or some other beneficial effect of the combination compared with the individual components.

The compounds of the present invention, and a chemotherapeutic agent or a radiosensitizer agent, utilized in combination therapy may be administered simultaneously, in either separate or combined formulations, or at different times e.g., sequentially, such that a combined effect is achieved. The amounts and regime of administration will be adjusted by the practitioner, by preferably initially lowering their standard doses and then titrating the results obtained.

The present invention also provides kits or single packages combining two or more active ingredients useful in treating cancer. A kit may provide (alone or in combination with a pharmaceutically acceptable diluent or carrier), the compound of the present invention and additionally at least one agent selected from the group consisting of a chemotherapeutic agent and a radiosensitizer agent (alone or in combination with diluent or carrier).

The present invention encompasses the preferred values listed below for radicals, substituents, and ranges; as well as any and all combinations of the preferred values for radicals, substituents, and ranges disclosed below. In addition, the specific and preferred values listed below for radicals, substituents, and ranges do not exclude other defined values or other values within defined ranges for the radicals and substituents.

[1] One embodiment of the present invention provides a pharmaceutical composition comprising: (1.) a radiolabeled pharmaceutical agent of the formula RI-Ch-L,-(BM)x, and (2.) an effective stabilizing amount of a compound of formula (I):

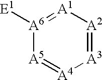

wherein

RI is ^{99m}Tc, ¹³¹I, ¹²⁵I, ¹²³I, ^{117m}Sn, ¹¹¹In, ⁹⁷Ru, ²⁰³Pb, ⁶⁷Ga, ⁶⁸Ga, ⁸⁹Zr, ⁹⁰Y, ¹⁷⁷Lu, ¹⁴⁹Pm, ¹⁵³Sm, ¹⁶⁶Ho, ¹³¹I, ³²P, ²¹¹At, ⁴⁷Sc, ¹⁰⁹Pd, ¹⁰⁵Rh, ¹⁸⁶Re, ¹⁸⁸Re, ⁶⁰Cu, ⁶²Cu, ⁶⁴Cu or ⁶⁷Cu;

Cₕ is a metal chelator or is a direct linkage (i.e., is absent);

Lₙ is a linking group or is a direct linkage (i.e., is absent);

each BM is independently a peptidomimetic or a non-peptide;

x is 1 to about 100;

E¹ is NH₂ or OH;

A¹, A², A³, A⁴ and A⁵ are each independently N, C(OH) or CR¹;

provided at least one of A¹, A², A³, A⁴ and A⁵ is not CH;

each R¹ is independently H, C(O)R , C(O)OR , NHC(═O)NHR², NHC(═S)NHR², OC(═O)R², OC(═O)OR², S(O)₂OR², C(O)NR³R⁴, C(O)NR³OR⁴, C(O)NR²NR³R⁴, NR³R⁴, NR³C(O)R⁴, PO(OR)(OR⁴), S(O)₂NR³R⁴, S(O)₂NR²NR³R⁴, S(O)₂NR³OR⁴, C₁-C₁₀ alkyl substituted with 0-5 R⁵, C₃-C₁₀ cycloalkyl substituted with 0-5 R⁵, C₂-C₁₀ alkenyl substituted with 0-5 R⁵, or aryl substituted with 0-5 R⁵;

R², R³, and R⁴ are each independently H, C₁-C₆ alkyl, C₃-C₆ cycloalkyl, C₁-C₆ alkenyl, benzyl, or phenyl; or R³ and R⁴ together form C₃-C₁₀ cycloalkyl or C₃-C₁₀ cycloalkenyl, optionally interrupted with O, S, NH, S(═O), S(O)₂, P(═O)(OH), C(═O)NH, NHC(═O), NHC(═O)NH, or NHC(═S)NH; and

each R⁵is independently H, NH₂, OH, CO₂H, C(═O)NH₂, C(═O)NHOH, C(═O)NHNH₂, NHC(═NH)NH₂, NHC(═O)NH₂, NHC(═S)NH₂, PO₃H₂, SO₃H, or S(O)₂NH₂;

or a pharmaceutically acceptable salt thereof.

[2] Another embodiment of the invention provides a pharmaceutical composition comprising: (1.) a radiolabeled pharmaceutical agent of the formula (II):

RI-Ch-Lₙ-(BM)ₓ (II); and

(2.) an effective stabilizing amount of a compound of formula (I):

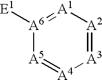

wherein

RI is ^{99m}Tc, ¹³¹I, ¹²⁵I, ¹²³I, ^{117m}Sn, ¹¹¹In, ⁹⁷Ru, ²⁰³Pb, ⁶⁷Ga, ⁶⁸Ga, ⁸⁹Zr, ⁹⁰Y, ¹⁷⁷Lu, ¹⁴⁹Pm, ¹⁵³Sm, ¹⁶⁶Ho, ¹³¹I, ³²P, ²¹¹At, ⁴⁷Sc, ¹⁰⁹Pd, ¹⁰⁵Rh, ¹⁸⁶Re, ¹⁸⁸Re, ⁶⁰Cu, ⁶²Cu, ⁶⁴Cu or ⁶⁷Cu;

Cₕ is a metal chelator or is a direct linkage (i.e., absent);

Lₙ is a linking group or is a direct linkage (i.e., absent);

each BM is independently an antibody, an antibody fragment, a peptide, a peptidomimetic, or a non-peptide;

x is 1 to about 10;

E¹ is NH₂ or OH;

A¹, A², A³, A⁴ and A⁵ are each independently N, C(OH) or CR¹;

provided at least one of A¹, A², A³, A⁴ and A⁵ is not CH;

each R¹ is independently H, C(O)R², C(O)OR², NHC(═O)NHR², NHC(═S)NHR², OC(═O)R², OC(═O)OR², S(O)₂OR², C(O)NR³R⁴, C(O)NR³OR⁴, C(O)NR²NR³R⁴, NR³R⁴, NR³C(O)R⁴, PO(OR³)(OR⁴), S(O)₂NR³R⁴, S(O)₂NR²NR³R⁴, S(O)₂NROR⁴, C₁-C₁₀ alkyl substituted with 0-5 R⁵, C₃-C₁₀ cycloalkyl substituted with 0-5 R⁵, C₂-C₁₀ alkenyl substituted with 0-5 R⁵, or aryl substituted with 0-5 R⁵;

R², R³, and R⁴ are each independently H, C₁-C₆ alkyl, C₃-C₆ cycloalkyl, C₁-C₆ alkenyl, benzyl, or phenyl; or R³ and R⁴ together form C₃-C₁₀ cycloalkyl or C₃-C₁₀ cycloalkenyl, optionally interrupted with O, S, NH, S(═O), S(O)₂, P(═O)(OH), C(═O)NH, NHC(═O), NHC(═O)NH, or NHC(═S)NH; and

each R⁵ is independently H, NH₂, OH, CO₂H, C(═O)NH₂, C(═O)NHOH, C(═O)NHNH₂, NHC(═NH)NH₂, NHC(═O)NH₂, NHC(═S)NH₂, PO₃H₂, SO₃H, or S(O)₂NH₂;

or a pharmaceutically acceptable salt thereof;

provided the compound of formula (I) is not (1) a substituted monohydroxyl aromatic compound; (2) a substituted dihydroxyl aromatic compound, in which the two hydroxyl groups are not adjacent to each other; (3) a substituted monohydroxyl-monoamino aromatic compound, in which the hydroxyl group and amino group are not adjacent to each other; or (4) an ortho, meta, or para aminobenzioc acid.

Another embodiment of the invention provides a pharmaceutical composition of the present invention wherein x is about 1 to about 100.

Another embodiment of the invention provides a pharmaceutical composition of the present invention wherein x is about 1 to about 50.

Another embodiment of the invention provides a pharmaceutical composition of the present invention wherein x is about 1 to about 25.

Another embodiment of the invention provides a pharmaceutical composition of the present invention wherein x is about 1 to about 10.

Another embodiment of the invention provides a pharmaceutical composition of the present invention wherein E¹ is NH₂.

Another embodiment of the invention provides a pharmaceutical composition of the present invention wherein E¹ is OH.

Another embodiment of the invention provides a pharmaceutical composition of the present invention wherein A¹ is C(OH) or CR¹.

Another embodiment of the invention provides a pharmaceutical composition of the present invention wherein A¹ is C(OH).

Another embodiment of the invention provides a pharmaceutical composition of the present invention wherein A¹ is CR¹.

Another embodiment of the invention provides a pharmaceutical composition of the present invention wherein A¹ is N.

Another embodiment of the invention provides a pharmaceutical composition of the present invention wherein A¹ is N, C(OH) or CR¹, wherein R¹ is not H.

Another embodiment of the invention provides a pharmaceutical composition of the present invention wherein A² is C(OH) or CR¹.

Another embodiment of the invention provides a pharmaceutical composition of the present invention wherein A¹ C(OH).

Another embodiment of the invention provides a pharmaceutical composition of the present invention wherein A² is CR¹.

Another embodiment of the invention provides a pharmaceutical composition of the present invention wherein A² is N.

Another embodiment of the invention provides a pharmaceutical composition of the present invention wherein A² is N, C(OH) or CR¹, wherein R¹ is not H.

Another embodiment of the invention provides a pharmaceutical composition of the present invention wherein A³ is C(OH) or CR¹.

Another embodiment of the invention provides a pharmaceutical composition of the present invention wherein A³ is C(OH).

Another embodiment of the invention provides a pharmaceutical composition of the present invention wherein A³ is CR¹.

Another embodiment of the invention provides a pharmaceutical composition of the present invention wherein A³ is N.

Another embodiment of the invention provides a pharmaceutical composition of the present invention wherein A³ is N, C(OH) or CR¹, wherein R¹ is not H.

Another embodiment of the invention provides a pharmaceutical composition of the present invention wherein A⁴ is C(OH) or CR¹.

Another embodiment of the invention provides a pharmaceutical composition of the present invention wherein A⁴ is C(OH).

Another embodiment of the invention provides a pharmaceutical composition of the present invention wherein A⁴ is CR¹.

Another embodiment of the invention provides a pharmaceutical composition of the present invention wherein A⁴ is N.

Another embodiment of the invention provides a pharmaceutical composition of the present invention wherein A⁴ is N, C(OH) or CR¹, wherein R¹ is not H.

Another embodiment of the invention provides a pharmaceutical composition of the present invention wherein A⁵ is C(OH) or CR¹.

Another embodiment of the invention provides a pharmaceutical composition of the present invention wherein A⁵ is C(OH).

Another embodiment of the invention provides a pharmaceutical composition of the present invention wherein A⁵ is CR¹.

Another embodiment of the invention provides a pharmaceutical composition of the present invention wherein A⁵ is N.

Another embodiment of the invention provides a pharmaceutical composition of the present invention wherein A⁵ is N or CR¹ (i.e., A⁵ is not C(OH)).

Another embodiment of the invention provides a pharmaceutical composition of the present invention wherein A⁵is N, C(OH) or CR¹, wherein R¹ is not H.

Another embodiment of the invention provides a pharmaceutical composition of the present invention wherein A⁶ is C(OH) or CR¹.

Another embodiment of the invention provides a pharmaceutical composition of the present invention wherein A⁶ is C(OH).

Another embodiment of the invention provides a pharmaceutical composition of the present invention wherein A⁶ is CR¹.

Another embodiment of the invention provides a pharmaceutical composition of the present invention wherein A⁶ is N.

Another embodiment of the invention provides a pharmaceutical composition of the present invention wherein A⁶ is N, C(OH) or CR¹, wherein R¹ is not H.

Another embodiment of the invention provides a pharmaceutical composition of the present invention wherein R¹ is H, C(O)R², C(O)OR², NHC(═O)NHR², NHC(═S)NHR, OC(═O)R, OC(═O)OR², S(O)₂OR², C(O)NR³R⁴, C(O)NR³OR⁴, C(O)NR²NR³R⁴, NR³R⁴, NR³C(O)R⁴, PO(OR³)(OR⁴), S(O)₂NR³R⁴, S(O)₂NR²NR³R⁴, S(O)₂NR³OR⁴, C₁-C₁₀ alkyl substituted with 0-3 R⁵, C₃-C₁₀ cycloalkyl substituted with 0-3 R⁵, C₂-C₁₀ alkenyl substituted with 0-3 R⁵, or aryl substituted with 0-5 R⁵.

Another embodiment of the invention provides a pharmaceutical composition of the present invention wherein R¹ is C(O)H, C(O)NH₂, C(O)NHNH₂, CO₂H, NHC(═O)NH₂, NHC(═S)NH₂, PO₃H₂, SO₃H, or S(O)₂NH₂.

Another embodiment of the invention provides a pharmaceutical composition of the present invention wherein R² is H, C₁-C₆ alkyl, C₃-C₆ cycloalkyl, benzyl, or phenyl.

Another embodiment of the invention provides a pharmaceutical composition of the present invention wherein R³ is H, C₁-C₆ alkyl, C₃-C₆ cycloalkyl, benzyl, or phenyl; or R³ and R⁴ together form C₃-C₁₀ cycloalkyl optionally interrupted with O, S, NH, S(═O), S(O)₂, P(═O)(OH), C(═O)NH, NHC(═O), NHC(═O)NH, or NHC(═S)NH.

Another embodiment of the invention provides a pharmaceutical composition of the present invention wherein R⁴ is H, C₁-C₆ alkyl, C₃-C₆ cycloalkyl, benzyl, or phenyl; or R³ and R⁴ together form C₃-C₁₀ cycloalkyl optionally interrupted with O, S, NH, S(═O), S(O)₂, P(═O)(OH), C(═O)NH, NHC(═O), NHC(═O)NH, or NHC(═S)NH.

Another embodiment of the invention provides a pharmaceutical composition of the present invention wherein R⁵ is H, NH₂, OH, CO₂H, C(═O)NH₂, PO₃H₂, SO₃H, or S(O)₂NH₂.

[3] A specific embodiment of the present invention is a composition of embodiment [1] or [2] wherein

E¹ is OH;

A¹, A², A³, and A⁴ are each independently C(OH) or CR¹;

A⁵ is C(OH);

each R¹ is independently H, C(O)R², C(O)OR², NHC(═O)NHR², NHC(═S)NHR², OC(═O)R², OC(═O)OR², S(O)₂OR², C(O)NR³R⁴, C(O)NR³OR⁴, C(O)NR²NR³R⁴, NR³R⁴, NR³C(O)R⁴, PO(OR³)(OR⁴), S(O)₂NR³R⁴, S(O)₂NR²NR³R⁴, S(O)₂NR³OR⁴, C₁-C₁₀ alkyl substituted with 0-3 R⁵, C₃-C₁₀ cycloalkyl substituted with 0-3 R⁵, C₂-C₁o alkenyl substituted with 0-3 R⁵, or aryl substituted with 0-5 R⁵;

R², R³, and R⁴ are each independently H, C₁-C₆ alkyl, C₃-C₆ cycloalkyl, benzyl, or phenyl; or R³ and R⁴ together form C₃-C₁₀ cycloalkyl optionally interrupted with O, S, NH, S(═O), S(O)₂, P(═O)(OH), C(═O)NH, NHC(═O), NHC(═O)NH, or NHC(═S)NH; and

each R⁵ is independently H, NH₂, OH, CO₂H, C(═O)NH₂, PO₃H₂, SO₃H, or S(O)₂NH₂;

or a pharmaceutically acceptable salt thereof.

[4] Another specific embodiment of the present invention is a composition of any one of embodiments [1] to [3] wherein

E¹ is OH;

A¹, A², and A³ are each independently C(OH) or CR¹;

A⁴ is C(OH);

A⁵is C(OH);

each R¹ is independently C(O)H, C(O)NH₂, C(O)NHNH₂, CO₂H, NHC(═O)NH₂, NHC(═S)NH₂, PO₃H₂, SO₃H, or S(O)₂NH₂;

or a pharmaceutically acceptable salt thereof.

[5] Another specific embodiment of the present invention is a composition of any one of embodiments [1] to [4] wherein

E¹ is NH₂;

A¹, A², A³, and A⁴ are each independently C(OH) or CR¹;

A⁵is C(OH);

each R¹ is independently H, C(O)R², C(O)OR², NHC(═O)NHR², NHC(═S)NHR², OC(═O)R², OC(═O)OR², S(O)₂OR², C(O)NR³R⁴, C(O)NR³OR⁴, C(O)NR²NR³R⁴, NR³R⁴, NR³C(O)R⁴, PO(OR³)(OR⁴), S(O)₂NR³R⁴, S(O)₂NR²NR³R⁴, S(O)₂NR³OR⁴, C₁-C₁₀ alkyl substituted with 0-3 R⁵, C₃-C₁₀ cycloalkyl substituted with 0-3 R⁵, C₂-C₁₀ alkenyl substituted with 0-3 R⁵, or aryl substituted with 0-5 R⁵;

R², R³, and R⁴ are each independently H, C₁-C₆ alkyl, C₃-C₆ cycloalkyl, benzyl, or phenyl; or R³ and R⁴ together form C₃-C₁₀ cycloalkyl optionally interrupted with O, S, NH, S(═O), S(O)₂, P(═O)(OH), C(═O)NH, NHC(═O), NHC(═O)NH, or NHC(═S)NH; and

each R⁵ is independently H, NH₂, OH, CO₂H, C(═O)NH₂, PO₃H₂, SO₃H, and S(O)₂NH₂;

or a pharmaceutically acceptable salt thereof.

[6] Another specific embodiment of the present invention is a composition of any one of embodiments [1] to [5] wherein

E¹ is NH₂;

A¹, A², A³, and A⁴ are each independently C(OH) or CR¹;

A⁵ is C(OH);

each R¹ is independently C(O)H, C(O)NH₂, C(O)NHNH₂, CO₂H, NHC(═O)NH₂, NHC(═S)NH₂, PO₃H₂, SO₃H, or S(O)₂NH₂;

or a pharmaceutically acceptable salt thereof.

[7] Another specific embodiment of the present invention is a composition of any one of embodiments [1] to [6] wherein

E¹ is NH₂ or OH;

A¹, A², A³, A⁴ and A⁵ are each independently N, C(OH) or CR¹, provided that A⁵ is not C(OH);

each R¹ is independently H, C(O)R², C(O)OR², NHC(═O)NHR², NHC(═S)NHR², OC(═O)R², OC(═O)OR², S(O)₂OR², C(O)NR³R⁴, C(O)NR³OR⁴, C(O)NR²NR³R⁴, NR³R⁴, NR³C(O)R⁴, PO(OR³)(OR⁴), S(O)₂NR³R⁴, S(O)₂NR²NR³R⁴, S(O)₂NR³OR⁴, C₁-C₁₀ alkyl substituted with 0-5 R⁵, C₃-C₁₀ cycloalkyl substituted with 0-5 R⁵, C₂-C₁₀ alkenyl substituted with 0-5 R⁵, or aryl substituted with 0-5 R⁵;

R², R³, and R⁴ are each independently H, C₁-C₆ alkyl, C₃-C₆ cycloalkyl, C₁-C₆ alkenyl, benzyl, or phenyl; or R³ and R⁴ together form C₃-C₁₀ cycloalkyl or C₃-C₁₀ cycloalkenyl optionally interrupted with O, S, NH, S(═O), S(O)₂, P(═O)(OH), C(═O)NH, NHC(═O), NHC(═O)NH, or NHC(═S)NH; and

each R⁵ is independently H, NH₂, OH, CO₂H, C(═O)NH₂, C(═O)NHOH, C(═O)NHNH₂, NHC(═NH)NH₂, NHC(═O)NH₂, NHC(═S)NH₂, PO₃H₂, SO₃H, or S(O)₂NH₂;

or a pharmaceutically acceptable salt thereof.

[8] Another specific embodiment of the present invention is a composition of any one of embodiments [1] to [7] wherein

E¹ is NH₂ or OH;

A¹, A², A³, A⁴, and A⁵ are each independently CR¹;

each R¹ is independently H, C(O)R², C(O)OR², NHC(═O)NHR², NHC(═S)NHR², OC(═O)R², OC(═O)OR², S(O)₂OR², C(O)NR³R⁴, C(O)NR³OR⁴, C(O)NR²NR³R⁴, NR³R⁴, NR³C(O)R⁴, PO(OR³)(OR⁴), S(O)₂NR³R⁴, S(O)₂NR²NR³R⁴, S(O)₂NR³OR⁴, C₁-C₁₀ alkyl substituted with 0-3 R⁵, C₃-C₁₀ cycloalkyl substituted with 0-3 R⁵, C₂-C₁₀ alkenyl substituted with 0-3 R⁵, or aryl substituted with 0-5 R⁵;

R², R³, and R⁴ are each independently H, C₁-C₆ alkyl, C₃-C₆ cycloalkyl, benzyl, or phenyl; or R³ and R⁴ together form C₃-C₁o cycloalkyl optionally interrupted with O, S, NH, S(═O), S(O)₂, P(═O)(OH), C(═O)NH, NHC(═O), NHC(═O)NH, or NHC(═S)NH; and

each R⁵ is independently H, NH₂, OH, CO₂H, C(═O)NH₂, PO₃H₂, SO₃H, or S(O)₂NH₂;

or a pharmaceutically acceptable salt thereof.

[9] Another specific embodiment of the present invention is a composition of any one of embodiments [1] to [8] wherein

E¹ is NH₂ or OH;

A¹, A², A³, A⁴, and A⁵ are each independently CR¹;

each R¹ is independently C(O)H, C(O)NH₂, C(O)NHNH₂, CO₂H, NHC(═O)NH₂, NHC(═S)NH₂, PO₃H₂, SO₃H, or S(O)₂NH₂;

or a pharmaceutically acceptable salt thereof.

[10] Another specific embodiment of the present invention is a composition of any one of embodiments [1] to [9] wherein the compound of formula (I) is not a substituted monohydroxyl aromatic compound, i.e., a compound of the formula:

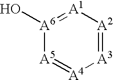

wherein

A¹, A², A³, A⁴ and A⁵ are each independently CR¹;

each R¹ is independently H, C(O)R², C(O)OR², NHC(═O)NHR², NHC(═S)NHR², OC(═O)R², OC(═O)OR², S(O)₂OR², C(O)NR³R⁴, C(O)NR OR⁴, C(O)NR²NR³R⁴, NR³R⁴, NR³C(O)R⁴, PO(OR³)(OR⁴), S(O)₂NR³R⁴, S(O)₂NR²NR³R⁴, S(O)₂NR³OR⁴, C₁-C₁₀ alkyl substituted with 0-5 R⁵, C₃-C₁₀ cycloalkyl substituted with 0-5 R⁵, C₂-C₁₀ alkenyl substituted with 0-5 R⁵, or aryl substituted with 0-5 R⁵;

R², R³, and R⁴ are each independently H, C₁-C₆ alkyl, C₃-C₆ cycloalkyl, C₁-C₆ alkenyl, benzyl, or phenyl; or R³ and R⁴ together form C₃-C₁₀ cycloalkyl or C₃-C₁₀ cycloalkenyl, optionally interrupted with O, S, NH, S(═O), S(O)₂, P(═O)(OH), C(═O)NH, NHC(═O), NHC(═O)NH, or NHC(═S)NH; and

each R⁵is independently H, NH₂, OH, CO₂H, C(═O)NH₂, C(═O)NHOH, C(═O)NHNH₂, NHC(═NH)NH₂, NHC(═O)NH₂, NHC(═S)NH₂, PO₃H₂, SO₃H, or S(O)₂NH₂;

or a pharmaceutically acceptable salt thereof.

[11] Another specific embodiment of the present invention is a composition of any one of embodiments [1] to [10] wherein the compound of formula (I) is not a substituted dihydroxyl aromatic compound, in which the two hydroxyl groups are not in the ortho position, i.e., a compound of the formula:

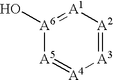

wherein

A¹, A², A³, A⁴ and A⁵ are each independently CR¹;

provided that any one of A², A³, and A⁴ is OH;

each R¹ is independently H, C(O)R², C(O)OR², NHC(═O)NHR², NHC(═S)NHR², OC(═O)R², OC(═O)OR², S(O)₂OR², C(O)NR³R⁴, C(O)NR³OR⁴, C(O)NR²NR³R⁴, NR³R⁴, NR³C(O)R⁴, PO(OR³)(OR⁴), S(O)₂NR³R⁴, S(O)₂NR²NR³R⁴, S(O)₂NR³OR⁴, C₁-C₁₀ alkyl substituted with 0-5 R⁵, C₃-C₁₀ cycloalkyl substituted with 0-5 R⁵, C₂-C₁₀ alkenyl substituted with 0-5 R⁵, or aryl substituted with 0-5 R⁵;

R², R³, and R⁴ are each independently H, C₁-C₆ alkyl, C₃-C₆ cycloalkyl, C₁-C₆ alkenyl, benzyl, or phenyl; or R³ and R⁴ together form C₃-C₁₀ cycloalkyl or C₃-C₁₀ cycloalkenyl, optionally interrupted with O, S, NH, S(═O), S(O)₂, P(═O)(OH), C(═O)NH, NHC(═O), NHC(═O)NH, or NHC(═S)NH; and

each R⁵ is independently H, NH₂, OH, CO₂H, C(═O)NH₂, C(═O)NHOH, C(═O)NHNH₂, NHC(═NH)NH₂, NHC(═O)NH₂, NHC(═S)NH₂, PO₃H₂, SO₃H, or S(O)₂NH₂;

or a pharmaceutically acceptable salt thereof.

[12] Another specific embodiment of the present invention is a composition of any one of embodiments [1] to [11] wherein the compound of formula (I) is not a substituted monohydroxyl-monoamino aromatic compound, in which the hydroxyl group and the arnino group are not in the ortho position, i.e., a compound of the formula:

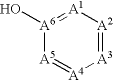

wherein

A¹, A², A³, A⁴ and A⁵ are each independently CR¹;

provided that any one of A², A³, and A⁴ is NH₂;

each R¹ is independently H, C(O)R², C(O)OR², NHC(═O)NHR², NHC(═S)NHR², OC(═O)R², OC(═O)OR², S(O)₂OR², C(O)NR³R⁴, C(O)NR³OR⁴, C(O)NR²NR³R⁴, NR³R⁴, NR³C(O)R⁴, PO(OR³)(OR⁴), S(O)₂NR³R⁴, S(O)₂NR²NR³R⁴, S(O)₂NR³OR⁴, C₁-C₁₀ alkyl substituted with 0-5 R⁵, C₃-C₁₀ cycloalkyl substituted with 0-5 R⁵, C₂-C₁₀ alkenyl substituted with 0-5 R⁵, or aryl substituted with 0-5 R⁵;

R², R³, and R⁴ are each independently H, C₁-C₆ alkyl, C₃-C₆ cycloalkyl, C₁-C₆ alkenyl, benzyl, or phenyl; or R³ and R⁴ together form C₃-C₁₀ cycloalkyl or C₃-C₁₀ cycloalkenyl, optionally interrupted with O, S, NH, S(═O), S(O)₂, P(═O)(OH), C(═O)NH, NHC(═O), NHC(═O)NH, or NHC(═S)NH; and

each R⁵ is independently H, NH₂, OH, CO₂H, C(═O)NH₂, C(═O)NHOH, C(═O)NHNH₂, NHC(═NH)NH₂, NHC(═O)NH₂, NHC(═S)NH₂, PO₃H₂, SO₃H, or S(O)₂NH₂;

or a pharmaceutically acceptable salt thereof.

[13] Another specific embodiment of the present invention is a composition of any one of embodiments [1] to [12] wherein the compound of formula (I) is not an ortho, meta, or para aminobenzioc acid, i.e., a compound of the formula:

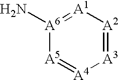

wherein

A¹, A², A³, A⁴ and A⁵ are each independently CH;

provided that any one of A¹, A², A³, A⁴ and A⁵ is COOH;

or a pharmaceutically acceptable salt thereof.

[14] Another specific embodiment of the present invention is a composition of any one of embodiments [1] to [13] wherein the compound of formula (I) is a compound of the formula:

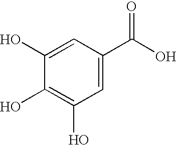

or a pharmaceutically acceptable salt thereof.

[15] Another specific embodiment of the present invention is a composition of any one of embodiments [1] to [14] wherein the compound of

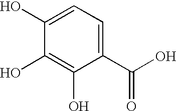

formula (I) is a compound of the formula:

or a pharmaceutically acceptable salt thereof.

[16] Another specific embodiment of the present invention is a composition of any one of embodiments [1] to [15] wherein the compound of formula (I) is compound of the formula:

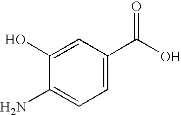

or a pharmaceutically acceptable salt thereof.

[17] Another specific embodiment of the present invention is a composition of any one of embodiments [1] to [16] wherein the compound of

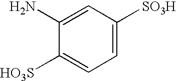

formula (I) is compound of the formula:

or a pharmaceutically acceptable salt thereof.

Metal Chelator

As used herein, a “metal chelator” (i.e., Cₕ) is a bifunctional chelator (BFC) employed to bind the metallic radionuclide to the pharmaceutical. Suitable metal chelator are disclosed, e.g., in Volkert, W. A. and Hoffman, T. J., Chem. Rev. 1999, 99, 2269-2292; Meeg, M. J. and Jurisson, S., Acc. Chem. Res., 1999, 32, 1053-1060; Kiu, S. And Edwards, D. S., Chem. Rev., 1999, 99, 2235-2268; Anderson, C. J. and Welch, M. J., Chem. Rev., 1999, 99, 2219-2234.

The metal chelator is selected to form stable complexes with the metallic radioisotope chosen for the particular application. Metal chelators for diagnostic radiopharmaceuticals are selected to form stable complexes with the metallic radioisotopes that have imageable garnma ray or positron emissions. In addition, metallic chelators for therapeutic radiopharmaceuticals are selected to form stable complexes with the metallic radioisotopes that have alpha particle, beta particle, Auger or Coster-Kronig electron emissions.

For example, metal chelators for technetium, copper and gallium isotopes are typically diaminedithiols, monoamine-monoamidedithiols, triamide-monothiols, monoamine-diamide-monothiols, diaminedioximes, or hydrazines. These metal chelators are generally tetradentate with donor atoms typically being nitrogen, oxygen or sulfur. These metal chelators will preferably have amine nitrogen and thiol sulfur donor atoms and hydrazine bonding units. The thiol sulfur atoms and the hydrazines may bear a protecting group which can be displaced either prior to using the reagent to synthesize a radiopharmaceutical or preferably in situ during the synthesis of the radiopharmaceutical.

[18] Another specific embodiment of the present invention is a composition of any one of embodiments [1] to [17] wherein the metal chelator (Cₕ) is a diaminedithiol, monoamine-monoamidedithiol, triamide-monothiol, monoamine-diamide-monothiol, diaminedioxime, or hydrazine.

Exemplary thiol protecting groups include those listed in Greene and Wuts, Protective Groups in Organic Synthesis, John Wiley & Sons, New York, 3rd Ed., 1999. Any thiol protecting group known in the art can be used. Examples of thiol protecting groups include, but are not limited to acetamidomethyl, benzamidomethyl, 1-ethoxyethyl, benzoyl, and triphenylmethyl.

Exemplary protecting groups for hydrazine bonding units are hydrazones which can be aldehyde or ketone hydrazones substituted with one or more alkyl, aryl and/or heterocycle. Particularly preferred hydrazones are described in U.S. Pat. No. 5,750,088, filed on Jun. 7, 1995.

The hydrazine bonding unit, when bound to a metallic radionuclide, is termed a hydrazido or diazenido group and serves as the point of attachment of the radionuclide to the remainder of the radiopharmaceutical. A diazenido group can be either terminal (only one atom of the group is bound to the metallic radionuclide) or can be a chelating diazenido group. In order to have a chelating diazenido group, at least one other atom of the group must also be bound to the radionuclide. The atom(s) bound to the metal are termed donor atom(s).

Metal chelators for yttrium, bismuth, and the lanthanide isotopes typically include cyclic and acyclic polyaminocarboxylates such as DTPA, DOTA, DO3A, 2-benzyl-DOTA, alpha-(2-phenethyl) 1,4,7,10-tetraazacyclododecane-1-acetic-4,7,10-tris(methylacetic)acid, 2-benzyl-cyclohexyldiethylenetriaminepentaacetic acid, 2-benzyl-6-methyl-DTPA, and 6,6″-bis[N,N,N″,N″-tetra(carboxymethyl)aminomethyl)-4′-(3-amino-4-methoxyphenyl)-2,2′:6′,2″-terpyridine. Procedures for synthesizing these metal chelators (i.e., those metallic chelators that are not commercially available) can be found, e.g., in Brechbiel, M. and Gansow, O., J. Chem. Soc. Perkin Trans. 1992, 1, 1175; Brechbiel, M. and Gansow, O., Bioconjugate Chem. 1991, 2, 187; Deshpande, S., et. al., J. Nucl. Med. 1990, 31, 473; Kruper, J., U.S. Pat. No. 5,064,956, and Toner, J., U.S. Pat. No. 4,859,777.

[18] Another specific embodiment of the present invention is a composition of any one of embodiments [1] to [17] wherein the metal chelator (Ch) is a cyclic or acyclic polyaminocarboxylate, such as DTPA, DOTA, DO3A, 2-benzyl-DOTA, alpha-(2-phenethyl) 1,4,7,10-tetraazacyclododecane-1-acetic-4,7,10-tris(methylacetic)acid, 2-benzyl-cyclohexyldiethylenetriaminepentaacetic acid, 2-benzyl-6-methyl-DTPA, and 6,6″-bis[N,N,N″,N″-tetra(carboxymethyl)aminomethyl)-4′-(3-amino-4-methoxyphenyl)-2,2′:6′,2″-terpyridine.

Metal chelators for rhenium, copper, palladium, platinum, iridium, rhodium, silver and gold isotopes typically include diaminedithiols, monoamine-monoamidedithiols, triamide-monothiols, monoamine-diamidemonothiols, diaminedioximes, and hydrazines.

[19] Another specific embodiment of the present invention is a composition of any one of embodiments [1] to [16] wherein the metal chelator (Cₕ) is a diaminedithiol, monoamine-monoamidedithiol, triamide-monothiol, monoamine-diamide-monothiol, diaminedioxime, or hydrazine.

When the radioisotope (RI) is a metallic radionuclide, the metal chelator (Cₕ) is typically present. When the radioisotope (RI) is a non-metallic radionuclide, the metal chelator (Cₕ) is typically absent (i.e., a direct linkage).

Metallic Radionuclide

As used herein, a “metallic radionuclide” is any suitable metallic radionuclide (i.e., radioisotope) useful in a therapeutic or diagnostic procedure in vivo or in vitro. The metallic radionuclide can be a metallic radioisotope that emits alpha particles, beta particles, gamma rays, positrons, or Auger electrons. The radiopharmaceuticals that include a gamma ray emitting isotope or positron emitting isotope are useful as imaging agents. The radiopharmaceuticals that include a beta particle, alpha particle or Auger electron emitting isotope are useful as therapeutic radiopharmaceuticals. The metallic radioisotope is complexed by the bifunctional metal chelator, which is attached directly or via a linker to one or more biologically active targeting molecules (biomolecules).

Suitable metallic radionuclides (i.e., metallic radioisotopes or metallic paramagnetic ions) include Antimony-124, Antimony-125, Arsenic-74, Barium-103, Barium-140, Beryllium-7, Bismuth-206, Bismuth-207, Bismuth-212, Cadmium-109, Cadmium-115m, Calcium-45, Cerium-1 39, Cerium-141, Cerium-144, Cesium-137, Chromium-51, Cobalt-55, Cobalt-56, Cobalt-57, Cobalt-58, Cobalt-60, Cobalt-64, Copper-60, Copper-62, Copper-64, Copper-67, Erbium-169, Europium-152, Gallium-64, Gallium-67, Gallium-68, Gadolinium-153, Gadolinium-157 Gold-195, Gold-199, Hafnium-175, Hafnium-175-181, Holmium-166, Indium-10, Indium-111, Iridium-192, Iron-55, Iron-59, Krypton-85, Lead-203, Lead-210, Lutetium-177, Manganese-54, Mercury-197, Mercury-203, Molybdenum-99, Neodymium-147, Neptunium-237, Nickel-63, Niobium-95, Osmium-185+191, Palladium-103, Palladium-109, Platinum-195m, Praseodymium-143, Promethium-147, Promethium-149, Protactinium-233, Radium-226, Rhenium-186, Rhenium-188, Rubidium-86, Ruthenium-97, Ruthenium-103, Ruthenium-105, Ruthenium-106, Samarium-153, Scandium-44, Scandium-46, Scandium-47, Selenium-75, Silver-110m, Silver-111, Sodium-22, Strontium-85, Strontium-89, Strontium-90, Sulfur-35, Tantalum-182, Technetium-99m, Tellurium-125, Tellurium-132, Thallium-204, Thorium-228, Thorium-232, Thallium-170, Tin-1 13, Tin-1 14, Tin-117m, Titanium-44, Tungsten-185, Vanadium-48, Vanadium-49, Ytterbium-169, Yttrium-86, Yttrium-88, Yttrium-90, Yttrium-91, Zinc-65, Zirconium-89, and Zirconium-95.

[20] Another specific embodiment of the present invention is a composition of any one of embodiments [1] to [19] wherein the radioisotope (RI) isTechnetium-99m(i.e., ^{99m}Tc), Tin-117m (i.e., ^{117m}Sn), Indium-111 (i.e., ¹¹¹In), Ruthenium-97 (i.e., ⁹⁷Ru), Lead-203 (i.e., ²⁰³Pb), Gallium-67 (i.e., ⁶⁷Ga), Gallium-68 (i.e., ⁶⁸Ga), Zirconium-89 (i.e., ⁸⁹Zr), Yttrium-90 (i.e., 90Y), Lutetium-177 (i.e., ⁷⁷Lu), Promethium-149 (i.e., ¹⁴⁹Pm), Samarium-153 (i.e., ¹⁵³S m), Holmium-166 (i.e., ¹⁶⁶Ho), Bismuth-212 (i.e., ²¹²Bi), Scandium-47 (i.e., ⁴⁷Sc), Palladium-109 (i.e., ¹⁰⁹Pd), Ruthenium-105 (i.e., ¹⁰⁵Rh), Rhenium-186 (i.e., ¹⁸⁶Re), Rhenium-188 (i.e., ¹⁸⁸Re), Copper-60 (i.e., ⁶⁰Cu), Copper-62 (i.e., ⁶²Cu), Copper-64 (i.e., ⁶⁴Cu), or Copper-67 (i.e., ⁶⁷Cu).

[21] Another specific embodiment of the present invention is a composition of any one of embodiments [1] to [19] wherein the radioisotope (RI) is Yttrium-90 (i.e., ⁹⁰Y) or Lutetium-177 (i.e., ¹⁷⁷Lu).

[22] Another specific embodiment of the present invention is a composition of any one of embodiments [1] to [21] wherein the radiopharmaceutical includes more than one metallic radionuclide.

Non-Metallic Radionuclide

As used herein, a “non-metallic radionuclide” is any suitable non-metallic radionuclide (i.e., radioisotope) useful in a therapeutic or diagnostic procedure in vivo or in vitro. The non-metallic radioisotope can emit alpha particles, beta particles, gamma rays, positrons, or Auger electrons. The radiopharmaceuticals that include a gamma ray emitting isotope or positron emitting isotope are useful as imaging agents. The radiopharmaceuticals that include a beta particle, alpha particle or Auger electron emitting isotope are useful as therapeutic radiopharmaceuticals. The non-metallic radioisotope can be covalently attached either directly to the biomolecule or to a linker. Suitable non-metallic radionuclides (i.e., metallic radioisotopes or metallic paramagnetic ions) include Iodine-131, Iodine-125, Iodine-123, Phosphorus-32, Astatine-211, Fluorine-18, Carbon-11, Oxygen-15, Bromine-76, and Nitrogen-13.

[23] Another specific embodiment of the present invention is a composition of any one of embodiments [1] to [22] wherein the radioisotope (RI) is Iodine-131 (i.e., ¹³¹I), Iodine-125 (i.e., ¹²⁵I), Iodine-123 (i.e., ¹²³I), Phosphorus-32 (i.e., ³²P), Astatine-211 (i.e., ²¹¹At), Fluorine-18 (i.e., ¹⁸F), Carbon-11 (i.e., ¹¹C), Oxygen-15 (i.e., ¹⁵O), or Nitrogen-13 (i.e., ¹³N).

[24] Another specific embodiment of the present invention is a composition of any one of embodiments [1] to [23] wherein the radiopharmaceutical includes more than one non-metallic radioisotope.

Linking Group

In addition to being directly linked to the metal chelator (Cₕ), the biomolecule (BM) can also be linked to the metal chelator (Cₕ) by a suitable linker (Lₙ). The linking group (Lₙ) can serve several roles. First, the linking group (Lₙ) can provide a spacing group between the non-metallic radionuclide and the one or more biomolecules (BM). Second the linking group (Lₙ) can provide a spacing group between the metal chelator (Cₕ), and the one or more biomolecules (BM). The spacing group can minimize the possibility that the radionuclide (RI) (e.g., non-metallic radionuclide or metallic radionuclide) will interfere with the interaction of the biomolecule (BM) with its biological target. The necessity of incorporating a linking group (Lₙ) in the biomolecule conjugate of the formula RI-Ch-Lₙ-(BM)ₓ is dependent on the identity of the biomolecule (BM), the radionuclide (RI), and the metal chelator (Cₕ), if present. For example, if the radionuclide (RI) and the optional metal chelator (Cₕ) cannot be attached to the biomolecule (BM) without substantially diminishing the biomolecule's affinity for its biological target, then a suitable linking group (Lₙ) can be used.

Third, a linking group (Lₙ) can provide a means of independently attaching multiple biomolecules (BMs) to one metal chelator, wherein the metal chelator includes one or more radionuclides. Specifically, up to 10 biomolecules (BMs) can be independently attached to one metal chelator.

Fourth, the linking group (Lₙ) can provide a means of incorporating a pharmacokinetic modifier into the radiopharmaceuticals of the present invention. The pharmacokinetic modifier serves to direct the biodistibution of the injected radiopharmaceutical other than by the interaction of the biomolecules (BM) with the biological target. The modifiers can be used to enhance or decrease hydrophilicity and to enhance or decrease the rate of blood clearance. The modifiers can also be used to direct the route of elimination of the pharmaceuticals. A wide variety of functional groups can serve as pharmacokinetic modifiers, including, but not limited to, carbohydrates, polyalkylene glycols, peptides or other polyamino acids, and cyclodextrins.

[25] Another specific embodiment of the present invention is a composition of any one of embodiments [1] to [24] wherein the linker (Lₙ) is a carbohydrate, cyclodextrin, polyalkylene glycol, amino acid, peptide (or other polyamino acid), or a poly(ethylene glycol) (PEG) chain.

[26] Another specific embodiment of the present invention is a composition of any one of embodiments [1] to [25] wherein the linker (Lₙ) separates the metal chelator (Cₕ) or the non-metallic radionuclide and the biomolecule (BM) by about 5 angstroms to about 200 angstroms, inclusive, in length.

[27] Another specific embodiment of the present invention is a composition of any one of embodiments [1] to [26] wherein the linker (Lₙ) separates the metal chelator (Cₕ) or the non-metallic radionuclide and the biomolecule (BM) by about 5 angstroms to about 100 angstroms, inclusive, in length.

[28] Another specific embodiment of the present invention is a composition of any one of embodiments [1] to [27] wherein the linker (Lₙ) separates the metal chelator (Cₕ) or the non-metallic radionuclide and the biomolecule (BM) by about 5 angstroms to about 50 angstroms, inclusive, in length.

[29] Another specific embodiment of the present invention is a composition of any one of embodiments [1] to [28] wherein the linker (Lₙ) separates the metal chelator (Cₕ) or the non-metallic radionuclide and the biomolecule (BM) by about 5 angstroms to about 25 angstroms, inclusive, in length.

Biomolecule

Each of the one or more biomolecules (BMs) can independently be a protein, antibody, antibody fragment, single chain antibody, polypeptide, oligonucleotide, peptide, peptidomimetic or non-peptide. Preferably, each of the one or more biomolecules is independently a peptide, peptidomimetic, or non-peptide of less than about 10,000 g/mol molecular weight. Exemplary preferred biomolecules (BMs) that can be part of the metallic radioisotope-metal chelator-linker-biomolecule conjugate or that can be part of the non-metallic radioisotope-linker-biomolecule conjugate include the following:

For the diagnosis of thromboembolic disorders or atherosclerosis, the biomolecule (BM) can be a cyclic IIb/IIIa receptor antagonist compound as described in U.S. Pat. No. 5,879,657; a RGD containing peptide as described in U.S. Pat. Nos. 4,578,079 or 4,792,525; PCT Application US88/04403, PCT US89/01742, PCT US90/03788, or PCT US91/02356; and as described by Ojima et. al., 204th Meeting of the Amer. Chem. Soc., 1992, Abstract 44; a peptide that is a fibrinogen receptor antagonist as described in European Patent Application 90202015.5, 90202030.4, 90202032.2, 90202032.0, 90311148.2, 90311151.6, or 90311537.6; a specific binding peptide or polypeptide described as a IIb/IIIa receptor ligand, a ligand for the polymerization site of fibrin, a laminin derivative, a ligand for fibrinogen or a thrombin ligand as described in PCT WO 93/23085 (excluding the technetium binding groups); an oligopeptide that corresponds to the IIIa protein described in PCT WO90/00178; a hirudin-based peptide as described in PCT WO90/03391; a IIb/IIIa receptor ligand as described in PCT WO90/15818; the thrombus platelet binding or atherosclerotic plaque binding peptide as described in PCT WO92/13572 (excluding the technetium binding group) or GB 9313965.7; a fibrin binding peptide as described in U.S. Pat. Nos. 4,427,646 or 5,270,030; a hirudin-based peptide as described in U.S. Pat. No. 5,279,812; a fibrin binding protein as described in U.S. Pat. No. 5,217,705; a guanine derivatives that binds to the IIb/IIIa receptor as described in U.S. Pat. No. 5,086,069; a tyrosine derivative as described in European Patent Application 0478328A1 or by Hartman et. al., J. Med. Chem., 1992, 35, 4640; or an oxidized low density lipoprotein (LDL).

For the diagnosis of infection, inflammation or transplant rejection, the biomolecule (BM) can be a leukocyte binding peptide as described in PCT WO93/17719 (excluding the technetium binding group), PCT WO92/13572 (excluding the technetium binding group) or U.S. Pat. No. 5,792,444 filed on Oct. 22, 1993; a chemotactic peptide as described in Eur. Pat. Appl. 90108734.6 or A. Fischman et. al., Semin. Nuc. Med., 1994, 24, 154; a leukostimulatory agent as described in U.S. Pat. No. 5,277,892; or an LTβ4 antagonist as described in WO 98/15295, filed on Oct. 3, 1997.

For the diagnosis of cancer, the biomolecule (BM) can be a somatostatin analog as described in UK Application 8927255.3 or PCT WO94/00489; a selectin binding peptide as described in PCT WO94/05269; a biological-function domain as described in PCT WO93/12819; Platelet Factor 4; or a growth factor (PDGF, VEGF, EGF, FGF, TNF MCSF or an interleukin Il1-8).

The biomolecule (BM) can also be a compound that binds a receptor that is expressed or upregulated in angiogenic tumor vasculature. For targeting the VEGF receptors, Flk-1/KDR, Flt-1, and neuropilin-1, the targeting moieties include peptides, polypeptides or peptidomimetics that bind with high affinity to the receptors. For example, peptides that include a 23 amino acid portion of the C-terminal domain of VEGF have been synthesized which competitively inhibit binding of VEGF to VEGFR (Soker, et. al., J. Biol. Chem., 1997, 272, 31582-8). Linear peptides of 11 to 23 amino acid residues that bind to the basic FGF receptor (bFGFR) are described by Cosic et. al., Mol. and Cell. Biochem., 1994, 130, 1-9. A preferred linear peptide antagonist of the bFGFR is the 16 amino acid peptide, Met-Trp-Tyr-Arg-Pro-Asp-Leu-Asp-Glu-Arg-Lys-GLₙ-GLₙ-Lys-Arg-Glu. Gho et. al. (Cancer Research, 1997, 57, 3733-40) describe the identification of small peptides that bind with high affinity to the angiogenin receptor on the surface of endothelial cells. A preferred peptide is Ala-GL,-Leu-Ala-Gly-Glu-Cys-Arg-Glu-Asn-Val-Cys-Met-Gly-lle-Glu-Gly-Arg, in which the two Cys residues form an intramolecular disulfide bond. Yayon et. al. (Proc. Natl. Acad. Sci, USA, 1993, 90, 10643-7) describe other linear peptide antagonists of FGFR, identified from a random phage-displayed peptide library. Two linear octapeptides, Ala-Pro-Ser-Gly-His-Tyr-Lys-Gly and Lys-Arg-Thr-Gly-GLₙ-Tyr-Lys-Leu are preferred for inhibiting binding of bFGF to it receptor.

Targeting moieties for integrins expressed in tumor vasculature include peptides, polypeptides and peptidomimetics that bind to αvβ3, αvβ5, α5β1, α4β1, α1β1, and α2β2. Pierschbacher and Rouslahti (J. Biol. Chem., 1987, 262, 17294-8) describe peptides that bind selectively to α5β1 and αvβ3. U.S. Pat. No. 5,536,814 describes peptides that bind with high affinity to the integrin α5β1. Burgess and Lim (J. Med. Chem., 1996, 39, 4520-6) disclose the synthesis of three peptides that bind with high affinity to αvβ3: cyclo[Arg-Gly-Asp-Arg-Gly-Asp], cyclo[Arg-Gly-Asp-Arg-Gly-D-Asp] and the linear peptide Arg-Gly-Asp-Arg-Gly-Asp. U.S. Pat. Nos. 5,770,565 and 5,766,591 disclose peptides that bind with high affinity to αvβ3. U.S. Pat. Nos. 5,767,071 and 5,780,426 disclose cyclic peptides that have an exocyclic Arg amino acid that have high affinity for αvβ3. Srivatsa et. al., (Cardiovascular Res., 1997, 36, 408-28) describe the cyclic peptide antagonist for αvβ3, cyclo[Ala-Arg-Gly-Asp-Mamb]. Tran et. al., (Bioorg. Med. Chem. Lett., 1997, 7, 997-1002) disclose the cyclic peptide cyclo[Arg-Gly-Asp-Val-Gly-Ser-BTD-Ser-Gly-Val-Ala] that binds with high affinity to αvβ3. Arap et. al. (Science, 1998, 279, 377-80) describe cyclic peptides that bind to αvβ3 and αvβ5, Cys-Asp-Cys-Arg-Gly-Asp-Cys-Phe-Cys, and cyclo[Cys-Asn-Gly-Asp-Cys]. Corbett et. al. (Biorg. Med. Chem. Lett., 1997, 7, 1371-6) describe a series of αvβ3 selective peptidomimetics. And Haubner et. al., (Angew. Chem., Int. Ed. Engl., 1997, 36, 1374-89) disclose peptides and peptidomimetic αvβ3 antagonists obtained from peptide libraries.

Alternative targeting moieties for tumor vasculature include compounds that interact with receptor tyrosine kinases. Receptor tyrosine kinases (TKs) are membrane proteins, which play a key role in the transduction of mitogenic signals across the cell to the nucleus (Rewcastle, G. W. et al, J. Med. Chem., 1995, 38, 3482-3487; Thompson, A. M. et al., J. Med. Clem., 1997, 40, 3915-3925). Of the many TKs that have been identified and characterized, those of the epidermal growth factor receptor (EGFR) family are particularly important, and have been implicated in a variety of ectopic cell proliferative processes. The over-expression of human EGF receptor is greatly amplified in several human tumors (Fry, D. W., Exp. Opin. Invest. Drugs, 1994, 3, 577-595; Jardines, L. et al., Pathobiology 1993, 61, 268-282), accompanied by an overphosphorylation of their protein targets. This increased phosphorylation of substrate tyrosine residues by oncogenic TK proteins is an essential step in the neoplastic transformation. Consequently, there has been great interest in developing inhibitors of TKs (TKIs) as anticancer drugs (Burke, T. R. Jr., Drugs Future, 1992, 17, 119-131; Chang, C. J. and Geahlen, R., J. Nat. Prod. 1992, 55, 1529-1560). The over-expression of EGF receptors in tumor cells also provides the foundation for the development of diagnostic and therapeutic radiopharmaceuticals by attaching a chelator and a radionuclide onto the TK receptor ligand (tyrosine kinase inhibitor).

Biomolecules (BMs) can also represent proteins, antibodies, antibody fragments, peptides, polypeptides, or peptidomimetics that bind to receptors or binding sites on other tissues, organs, enzymes or fluids. Examples include the β-amyloid proteins that have been demonstrated to accumulate in patients with Alzheimer's disease, atrial naturetic factor derived peptides that bind to myocardial and renal receptors, antimyosin antibodies that bind to areas of infarcted tissues, or nitroimidazole derivatives that localize in hypoxic areas in vivo.

[30] Another specific embodiment of the present invention is a composition of any one of embodiments [1] to [29] wherein the biomolecule (BM) is an antibody, an antibody fragment, a peptide, a peptidometic, or a non-peptide.

[31] Another specific embodiment of the present invention is a composition of any one of embodiments [1] to [29] wherein the biomolecule (BM) is a peptidometic or a non-peptide.

[32] Another specific embodiment of the present invention is a composition of any one of embodiments [1] to [29] wherein the biomolecule (BM) is an antibody fragment.

[33] Another specific embodiment of the present invention is a composition of any one of embodiments [1] to [29] wherein the biomolecule (BM) is a peptidometic.

[34] Another specific embodiment of the present invention is a composition of any one of embodiments [1] to [29] wherein the biomolecule (BM) is a non-peptide.

Combination Therapy

Another aspect of the present invention contemplates the combination of chemotherapeutics and angiogenesis-targeted therapeutic radiopharmaceuticals of the present invention. The combination of chemotherapeutics and angiogenesis-targeted therapeutic radiopharmaceuticals of the present invention can effectively target the luminal side of the neovasculature of tumors. Such targeting can provide a surprising and enhanced degree of tumor suppression, relative to each treatment modality alone without significant additive toxicity.

Another aspect of the present invention contemplates the compounds of the present invention (e.g., a compound comprising: a targeting moiety and a chelator). Specifically, the targeting moiety bound to the chelator is a benzodiazepine, benzodiazepinedione, or dibenzotrihydroannulene nonpeptide. In addition, the targeting moiety binds to a receptor that is upregulated during angiogenesis. The compound has 0-1 linking groups between the targeting moiety and chelator. Moreover, the compound is administered in combination therapy, with one or more suitable chemotherapeutic agent(s) (e.g., mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma, colony stimulating factor-1, colony stimulating factor-2, denileukin diftitox, interleukin-2, and/or leutinizing hormone releasing factor).

This combination therapy may further, optionally, include a suitable radiosensitizer agent, or a pharmaceutically acceptable salt thereof, to enhance the radiotherapeutic effect together with the chemotherapeutic agent. Suitable radiosensitizer agents include, e.g., 2-(3-nitro-1,2,4-triazol-1-yl)-N-(2-methoxyethyl)acetamide, N-(3-nitro-4-quinolinyl)-4-morpholinecarboxamidine, 3-amino-1,2,4-benzotriazine-1,4-dioxide, N-(2-hydroxyethyl)-2-nitroimidazole-1-acetamide, 1-(2-nitroimidazol-1-yl)-3-(1-piperidinyl)-2-propanol, and 1-(2-nitro-1-imidazolyl)-3-(1-aziridino)-2-propanol. A discussion of radiosensitizer agents is provided in Rowinsky-EK, Oncology-Huntingt., October 1999; 13(10 Suppl 5): 61-70; Chen-A Y et al., Oncology-Huntingt. October 1999; 13 (10 Suppl 5): 39-46; Choy-H, Oncology-Huntingt. October 1999; 13 (10 Suppl 5): 23-38; and Herscher-L L et al, Oncology-Huntingt. October 1999; 13 (10 Suppl 5): 11-22.

It is a further aspect of the invention to provide kits having a plurality of active ingredients (with or without carrier) which, together, may be effectively utilized for carrying out the novel combination therapies of the invention.

It is another aspect of the invention to provide a novel pharmaceutical composition which is effective, in and of itself, for utilization in a beneficial combination therapy because it includes compounds of the present invention, and a chemotherapeutic agent or a radiosensitizer agent, which may be utilized in accordance with the invention.

In another aspect of the invention, the present invention provides a method for treating cancer in a patient in need of such treatment. The method includes the steps of administering a therapeutically effective amount of a compound of the present invention to the patient and administering a therapeutically effective amount of at least one agent selected from the group consisting of a chemotherapeutic agent and a radiosensitizer agent, to the patient.

Additional Stabilizers

The radiopharmaceutical composition of the present invention can optionally include additional (i.e., one or more) stabilizers that can effectively stabilize the radiopharmaceutical composition. As such, one or more additional suitable stabilizers can be employed to stabilize the radiopharmaceutical composition. Suitable additional stabilizers include, e.g., ascorbic acid, benzyl alcohol, gentisic acid, an ester of gentisic acid, gentisyl alcohol, an ester of gentisyl alcohol, p-aminobenzoic acid, cystamine, cystamine, 5-amino-2-hydroxybenzoic acid, nicotinic acid, nicotinamide, propylene glycol, dextran, inositol, a compound of formula (I), as defined herein, or a pharmaceutically acceptable salt thereof; as well as stabilizers disclosed in U.S. Pat. Nos. 5,961,955; 5,384,113; 4,497,744; 4,332,000; and PCT/US94/06276.

[35] Another specific embodiment of the present invention is a composition of any one of embodiments [1] to [34] further comprising additional (e.g., one or more) stabilizers selected from the group consisting of ascorbic acid, benzyl alcohol, gentisic acid, an ester of gentisic acid, gentisyl alcohol, an ester of gentisyl alcohol, p-aminobenzoic acid, cystamine, cystamine, 5-amino-2-hydroxybenzoic acid, nicotinic acid, nicotinamide, propylene glycol, dextran, inositol, a compound of formula (I), as defined herein, a pharmaceutically acceptable salt of a compound of formula (I); a stabilizer disclosed in U.S. Pat. No. 5,961,955; a stabilizer disclosed in U.S. Pat. No. 5,384,113; a stabilizer disclosed in U.S. Pat. No. 4,497,744; a stabilizer disclosed in U.S. Pat. No. 4,332,000; and a stabilizer disclosed in PCT/US94/06276.

[36] Another specific embodiment of the present invention is a composition of embodiment [35] wherein each of the additional stabilizers are present in the composition at a concentration of about 0.1 mg/mL to about 20 mg/mL.

Ascorbic acid is known as vitamin C, and is a commonly used antioxidant to prevent radiolytic decomposition of ^{99m}Tc and ^{186/188}Re radiopharmaceuticals (WO95/33757; Anticancer Res. 1997, 17, 1783-1796; U.S. Pat. Nos. 5,093,105 and 5,306,482) or radiolabeled peptides (U.S. Pat. Nos. 5,393,512; 5,384,113; and 5,961,955). Ascorbic acid is readily available GRAS (generally recognized as safe) substance often used in pharmaceutical compositions and other formulations used for biological purposes and may be used at levels as high as about 10 mg/mL of the final formulation.

Specifically, the use of about 1-2 mg/mL of a polyhydroxylated aromatic compound such as 2,3,4-trihydroxylbenzoic acid or 3,4,5-trihydroxylbenzoic acid and about 10-20 mg/mL of a second stabilizer such as ascorbic acid or gentisic acid is particularly useful in some of the radiopharmaceutical compositions disclosed herein.

The combinational use of two different stabilizers is especially useful for stabilization of therapeutic radiopharmacutical compositions. There is a high level of radioactivity contained in the bulk formulation in comparison to the level in an individual vial. When working in bulk, the deposition of β-energy from radionuclide is much higher than small volume, i.e. there is increased deposition, and thus greater potential for damage to the radiolabeled biomolecule, per unit volume. Better stabilization may therefore be needed. An increase in the concentration of a polyhydroxylated aromatic compound or a water-soluble aromatic amine in this instance may not be desirable due to high “osmolarity”. Therefore, a second (or third) stabilizer is needed to provide increased stabilization for the bulk therapeutic radiopharmaceutical composition. Secondary (or tertiary) stabilizers are preferentially utilized in combination with a polyhydroxylated aromatic compound or a water-soluble aromatic amine when large amount of activity (>100 mCi) or a large activity concentration (>5 mCi/mL) is desired.

One major advantage of using the stabilizing agents disclosed herein is that the prepared radiopharrnaceutical composition can typically be stored for several hours to several days, while maintaining the RCP (>90%) of the radiopharmaceutical. A stabilizer is introduced to the therapeutic radiopharmaceutical preparation, which is preferably in aqueous solution suitable for in vivo administration to human patients. The addition of the stabilizer can be prelabeling or post labeling.

The integrity of a radiopharmaceutical is measured by the radiochemical purity (RCP) of the radiolabeled compound using ITLC or more preferably HPLC. The advantage of using HPLC is that radio-impurities caused by radiolytic degradation can be separated from the radiopharmaceutical under optimized chromatographic conditions. inproved stability over time for radiopharmaceutical compositions of this invention can be demonstrated by determining the change in RCP of the radiolabeled compound in samples taken at representative time points. The radiopharmaceutical compositions of this invention are effective in maintaining the long-term stability of samples that have been frozen, thawed, and re-tested up to 7 day post-labeling.

The initial RCP of a radiopharmaceutical is largely dependent on radiolabeling conditions such as pH, heating temperature and time. Once a radiopharmaceutical is prepared in high yield, the ability of an antioxidant to stabilize a radiopharmaceutical composition is measured by the RCP change over a certain period of time. For comparison purposes, sodium gentisate or sodium ascorbate was often used as the reference since they are known stabilizers for various radiopharmaceutical formulations of compositions.

The therapeutic radiopharmaceutical compositions are preferably stored at low temperatures (e.g., −20° C. or colder) to avoid extensive radiolysis during release and transportation. The amount of the stabilizer used in the therapeutic radiopharmaceutical composition can be adjusted according to the sensitivity of a specific radiolabeled compound towards radiolytic decomposition. In addition, the storage and/or transportation temperature of he therapeutic radiopharmaceutical composition can be adjusted according to the sensitivity of a specific radiolabeled compound towards radiolytic decomposition.

The therapeutic radiopharmaceutical compositions are preferably stored in a container having a head space. In one embodiment, the head space can typically be a vacuum or a partial vacuum (e.g., the atmospheric pressure of the head space can be from below about 1 atm. down to about 0 atm.). In an alternative embodiment, the atmospheric pressure of the head space can be from about 1 atm. to about 2 atm. When a gas is present in the head space, the gas will typically be an inert gas (e.g., helium, nitrogen, argon, etc.) or a combination thereof. It is preferred that the head space include little or no appreciable amounts of oxygen or unstable forms thereof (e.g., ozone). The absence of oxygen and unstable forms thereof will typically diminish the occurrence of radiolysis during release and transportation. In addition, the pressure of the head space as well as the presence or absence of specific inert gases during the storage and/or transportation can be adjusted according to the sensitivity of a specific radiolabeled compound towards radiolytic decomposition.

The radiopharmaceutical composition can optionally include one or more buffers. Any suitable buffer can be employed, provided the buffer can effectively maintain the desired pH range of the radiopharmaceutical composition over an extended period of time (e.g., up to several hours, up to a day, up to a week, or up to a month). Preferably, the anion is not a phosphate, carbonate, oxalate, or sulfate. It is also preferred that the cation is not a metallic ion. In addition, it is preferred that the buffer maintain the pH of the composition between about 4 and about 8. Suitable buffers include, e.g., ammonium acetate, tetraalkyl ammonium acetate, ammonium citrate, amino acids such as lysine and glycine, tris buffers, and triane buffers such as dicine buffer and bicine buffer.

The stable radiopharmaceutical compositions of the present invention are suitable for treating cancer, a thromboembolic disorder, atherosclerosis, an infection, inflammation, transplant rejection or a disease state that is associated with the following receptors: a cyclic IIb/IIIa receptor, a fibrinogen receptor, a myocardial receptor, a renal receptor, LTβ4, selectin, growth factor (PDGF, VEGF, EGF, FGF, TNF MCSF or an interleukin Il1-8), a receptor that is expressed or upregulated in angiogenic tumor vasculature, αvβ3, αvβ5, α5β1, α4β1, α1β1, or α2β2, α5β1, αvβ3, α5β1, or tyrosine kinases (e.g., epidermal growth factor receptor (EGFR) family in a mammal (e.g., human). In addition, the stable radiopharmaceutical compositions of the present invention are suitable for treating and/or imaging a tumor in or on a mammalian (e.g., human) tissue, in vivo or in vitro. As such, the stable radiopharmaceutical compositions of the present invention are suitable for treating and/or imaging a tumor in a mammal. The tumor can be located in any part of the mammal. Specifically, the tumor can be located in the breast, lung, thyroid, lymph node, genitourinary system (e.g., kidney, ureter, bladder, ovary, teste, or prostate), musculoskeletal system (e.g., bones, skeletal muscle, or bone marrow), gastrointestinal tract (e.g., stomach, esophagus, small bowel, colon, rectum, pancreas, liver, or smooth muscle), central or peripheral nervous system (e.g., brain, spinal cord, or nerves), head and neck tumors (e.g., ears, eyes, nasopharynx, oropharynx, or salivary glands), or the heart.

The radiopharmaceutical compositions of the present invention can be administered to a mammalian host, such as a human patient in a variety of forms adapted to the chosen route of administration, i.e., orally or parenterally (e.g., by intravenous, intramuscular, intraperitoneal). Preferably, the compounds are administered parenterally.

The radiopharmaceutical compositions may also be administered intravenously or intraperitoneally by infusion or injection. Solutions of the radiopharmaceutical compositions can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the radiopharmaceutical compositions which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the radiopharmaceutical composition in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the radiopharmaceutical composition plus any additional desired ingredient present in the previously sterile-filtered solutions.

For illustration, suitable concentrations of the radiopharmaceutical composition for use in therapy or diagnosis include concentrations of each of the stabilizers in the range of from about 0.1 to about 20 mg/mL As such, the compound of formula (I) can be present in the radiopharmaceutical composition at a concentration from about 0.1 mg/mL to about 20 mg/mL In addition, each additional stabilizer can be present in the radiopharmaceutical composition at a concentration from about 0.1 mg/mL to about 20 mg/mL.

For illustration, all of the radioisotopes (e.g., metallic and/or non-metallic radionuclides) of the radiopharmaceutical composition, for use in therapy or diagnosis, can be present such that the amount of radioactivity is about 10 mCi to about 2000 mCi, about 10 mCi to about 1000 mCi, or about 25 mCi to about 250 mCi. In addition, all of the radioisotopes (e.g., metallic and/or non-metallic radionuclides) of the radiopharmaceutical composition, for use in therapy or diagnosis, can be present at a combined concentration of greater than about 5 mCi/nL of the radiopharmaceutical composition.

[37] Another specific embodiment of the present invention is a composition of any one of embodiments [1] to [36] wherein all of the radioisotopes (e.g., metallic and/or non-metallic radionuclides) of the radiopharmaceutical composition, for use in therapy or diagnosis are present such that the amount of radioactivity is about 10 mCi to about 2000 mCi

[38] Another specific embodiment of the present invention is a composition of any one of embodiments [1] to [37] wherein all of the radioisotopes (e.g., metallic and/or non-metallic radionuclides) of the radiopharmaceutical composition, for use in therapy or diagnosis are present such that the amount of radioactivity is about 10 mCi to about 1000 mCi.

[39] Another specific embodiment of the present invention is a composition of any one of embodiments [1] to [38] wherein all of the radioisotopes (e.g., metallic and/or non-metallic radionuclides) of the radiopharmaceutical composition, for use in therapy or diagnosis, are present such that the amount of radioactivity is about 25 mCi to about 250 mCi.

[40] Another specific embodiment of the present invention is a composition of any one of embodiments [1] to [39] wherein all of the radioisotopes (e.g., metallic and/or non-metallic radionuclides) of the radiopharmaceutical composition, for use in therapy or diagnosis, are present at a combined concentration of greater than about 5 mCi/mL of the radiopharmaceutical composition.

[41] Another specific embodiment of the present invention is a composition of any one of embodiments [1] to [40] wherein any one of the radioisotopes (e.g., metallic and/or non-metallic radionuclides) of the radiopharmaceutical composition, for use in therapy or diagnosis, is ⁹⁰Y or ¹⁷⁷Lu.

[42] Another specific embodiment of the present invention is a composition of any one of embodiments [1] to [41] wherein each of the radioisotopes of the radiopharmaceutical composition, for use in therapy or diagnosis, are ⁹⁰Y or ¹⁷⁷Lu.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The compounds are preferably dissolved or dispersed in a nontoxic liquid vehicle, such as physiological saline or a similar aqueous vehicle, to the desired concentration. A preselected therapeutic unit dose is then administered to the test animal or human patient, by oral administration or ingestion or by parenteral administration, as by intravenous or intraperitoneal infusion or injection, to attain the desired in vivo concentration. Doses useful for treating or imaging the desired disease can be derived from those found to be effective to treat or image the diseases in humans in vitro or in animal models, or from dosages of other compounds, previously employed in animal therapy or imaging.

It is appreciated that certain features of the invention, which are, for clarity, described in the context of separate embodiments, may also be provided in combination in a single embodiment. Conversely, various features of the invention which are for brevity, described in the context of a single embodiment, may also be provided separately or in any subcombination.

The invention will now be illustrated by the following non-limiting examples.

### EXAMPLES

The integrity of a radiopharmaceutical is measured by the radiochemical purity (RCP) of the radiolabeled compound using ITLC or more preferably HPLC. The advantage of using HPLC is that radio-impurities caused by radiolytic degradation can be separated from the radiopharmaceutical under optimized chromatographic conditions. Improved stability over time for radiopharmaceutical compositions of this invention can be demonstrated by determining the change in RCP of the radiolabeled compound in samples taken at representative time points. The radiopharmaceutical compositions of this invention are effective in maintaining the long-term stability of samples that have been frozen, thawed, and re-tested periodically for 7 days.

The initial RCP of a radiopharmaceutical is largely dependent on radiolabeling conditions such as pH, heating temperature and time. Once a radiopharmaceutical is prepared in high yield, the stability of the radiopharmaceutical composition is measured by the` RCP change of the radiopharmaceutical over a certain period of time. For comparison purposes, sodium gentisate or sodium ascorbate was often used as the reference since they have been used in various radiopharmaceutical formulations.

Materials. Acetic acid (ultra-pure), ammonium hydroxide (ultra-pure), ascorbic acid (sodium salt), diethylenetriaminepentaacetic acid (DTPA), 3-hydroxy-4-aminobenzoic acid, 2,3,4-trihydroxybenzoic acid, 3,4,5-trihydroxybenzoic acid, 3,4,5-trihydroxybenzaldehyde, and sodium gentisate were purchased from either Aldrich or Sigma Chemical Co., and were used as received. ⁹⁰YCl₃ (in 0.05 N HCl) was purchased from NEN Life Sciences, N. Billerica, Mass. High specific activity ¹⁷⁷LuCl₃ was obtained from University of Missouri Research Reactor, Columbia, Mo.

Analytical Methods. HPLC method 1 used a HP-1100 HPLC system with a UV/visible detector (λ=220 nm), an IN-US radio-detector, and a Zorbax C₁₈ column (4.6 mm×250 mm, 80 Å pore size). The flow rate was 1 mL/min with the mobile phase being isocratic from 0 to 18 min using 87% solvent A (0.025 M ammonium acetate buffer, pH 6.8) and 13% solvent B (acetonitrile), followed by an isocratic wash using 40% of solvent A and 60% solvent B from 19 to 25 min.

HPLC method 2 used a HP-1100 HPLC system with a UV/visible detector (λ=220 nm), an IN-US radio-detector, and a Zorbax C₁₈ column (4.6 mm×250 mm, 80 Å pore size). The flow rate was 1 mL/min with a gradient mobile phase starting from 92% solvent A (0.025 M ammonium acetate buffer, pH 6.8) and 8% solvent B (acetonitrile) at 0 min to 90% solvent A and 10% solvent B at 18 min, followed by an isocratic wash using 40% of solvent A and 60% solvent B from 19 to 25 min.

HPLC method 3 used a HP-1100 HPLC system with a UV/visible detector (λ=220 nm), an IN-US radio-detector, and a Zorbax C₁₈ column (4.6 mm×250 mm, 80 Å pore size). The flow rate was 1 mL/min with a gradient mobile phase starting from 90% solvent A (0.025 M ammonium acetate buffer, pH 6.8) and 10% solvent B (acetonitrile) at 0 min to 88% solvent A and 12% solvent B at 18 min, followed by an isocratic wash using 40% of solvent A and 60% solvent B from 19 to 25 min.

The ITLC method used Gelman Sciences silica-gel ITLC paper strips and 1:1 mixture of acetone and saline as eluant. By this method, the radiolabeled compounds migrate to the solvent front while [⁹⁰Y]/¹⁷⁷Lu]colloid and [⁹⁰Y]/¹⁷⁷Lu]acetate remain at the origin.

In the following examples of stable radiopharmaceutical compositions of the present invention, the chelator-optional linker-biomolecule conjugates are:

Compound A

2-(1,4,7,10-tetraaza-4,7,10-tris(carboxymethyl)-1-cyclododecyl)acetyl-Glu(cyclo{Lys-Arg-Gly-Asp-D-Phe})-cyclo{Lys-Arg-Gly-Asp-D-Phe}

Compound B

2-[({4-[3-(N-{2-[(2R)-2-((2R)-3-Sulfo-2-{2-[1,4,7,10-tetraaza-4,7,10-tris (carboxymethyl)cyclododecyl]acetylamino}propyl)-3-sulfopropyl]ethyl}carbamoyl)propoxy]-2,6-dimethylphenyl}sulfonyl)amino](2S)-3-({7-[(imidazol-2-ylamino)methyl]-1-methyl-4-oxo(3-hydroquinolyl)}carbonylamino)propanoic Acid

Compound C

DOTA/2-{[(4-{3-[N-(2-{(2R)-2-[(2R)-2-(4-{N-[(1R)-1-(N-{(1R)-1-[N-(2-{4-[4-({[(1S)-1-Carboxy-2-({7-[(imidazol-2-ylamino)methyl]-1-methyl-4-oxo(3-hydroquinolyl)}carbonylamino)-1-carboxyethyl]amino}-sulfonyl)-3,5-dimethylphenoxy]butanoylamino}ethyl)carbamoyl]-2-sulfoethyl}carbamoyl)-2-sulfoethyl]carbamoyl}(2S)-2-aminobutanoylamino)-3-sulfopropyl]-3-sulfopropyl}ethyl)carbamoyl]propoxy}-2,6-dimethylphenyl)sulfonyl]amino}(2S)-3-({7-[(imidazol-2-ylamino)methyl]-1-methyl-4-oxo(3-hydroquinolyl)}carbonylamino)propanoic Acid Conjugate.

The syntheses of these conjugates are described in PCT Application No. WO 0035492.

Compound A

Synthesis of 2-(1,4,7,10-tetraaza-4,7,10-tris(carboxymethyl)-1-cyclododecyl)acetyl-Glu(cyclo{Lys-Arg-Gly-Asp-D-Phe})-cyclo{Lys-Arg-Gly-Asp-D-Phe}

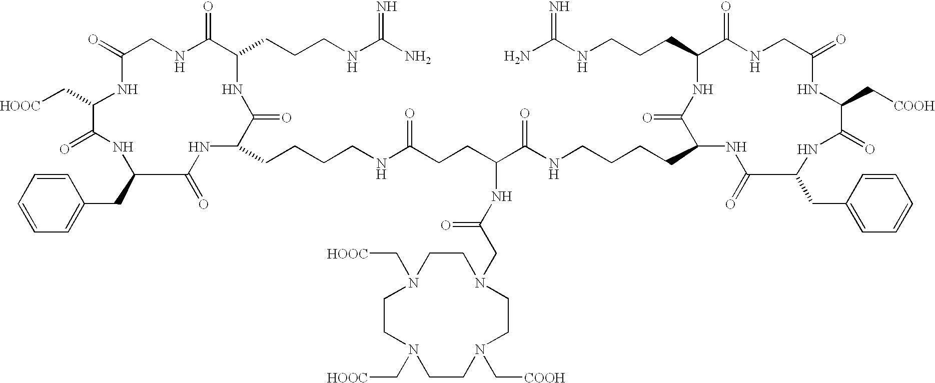

General Procedure for Solid Phase Peptide Synthesis Using Boc-Chemistry on the Oxime Resin for the Preparation of Cyclic Peptides

The appropriately protected cyclic peptides, described in the Examples, were prepared by manual solid phase peptide synthesis using Boc-teabag chemistry (Houghton, 1985) on a p-nitrobenzophenone oxime solid support (DeGrado, 1982, Scarr and Findeis, 1990). The 5.0 cm×5.0 cm teabags were made from 0.75 mm mesh polypropylene filters (Spectra Filters) and filled with 0.5 g (or 1 g) of the oxime resin. The coupling and deprotection steps were carried out in a polypropylene reactor using a table-top shaker for agitation. Synthesis of the protected pentapeptide-resin intermediate was achieved by first coupling Boc-Gly-OH to the oxime resin (substitution 0.69 mmol/g or 0.95 mmol/g). Attachment of Boc-Gly-OH onto the oxime resin was achieved by using five equivalents each of the amino acid, HBTU and diisopropylethylamine (DIPEA) in DMF. Coupling of the first amino acid generally occurred over 2-3 days. After thorough washing, substitution levels were determined using the picric acid assay (Stewart and Martin). Unreacted oxime groups on the resin were then capped with a solution of DIPEA and trimethylacetyl chloride in DMF. The boc-group was deprotected using 50% or 25% TFA in DCM (30 min). Coupling of the other protected boc-amino acids were performed in a similar manner by overnight shaking (1-2 days), and the coupling yields for each newly added amino acid was determined using the picric acid assay.

Part A. Preparation of Cyclo{Arg(Tos)-Gly-Asp(OBzl)-D-Phe-Lys(Cbz)}

The N-terminus Boc-protecting group of the peptide sequence Boc-Asp(OBzl)-D-Phe-Lys(Z)-Arg(Tos)-Gly-Oxime resin was removed using standard deprotection (25% TFA in CH₂Cl₂). After eight washes with DCM, the resin was treated with 10% DIEA/DCM (2×10 min.). The resin was subsequently washed with DCM (×5) and dried under high vacuum. The resin (1.7053 g, 0.44 mmol/g) was then suspended in dimethylformamide (15 mL). Glacial acetic acid (43.0 μL, 0.750 mmol) was added, and the reaction was heated to 60_C for 72 h. The resin was filtered, and washed with DMF (2×10 mL). The filtrate was concentrated to an oil under high vacuum. The resulting oil was triturated with ethyl acetate. The solid thus obtained was filtered, washed with ethyl acetate, and dried under high vacuum to give 510.3 mg of the desired product. ESMS: Calcd. for C₄₉H₅₉N₉O₁₁S, 981.40. Found, 982.6 [M+H]+1. Analytical HPLC, Method 1A, Rₜ=15.574 min, Purity=89%.

Part B. Preparation of cyclo{Arg-Gly-Asp-D-Phe-Lys}

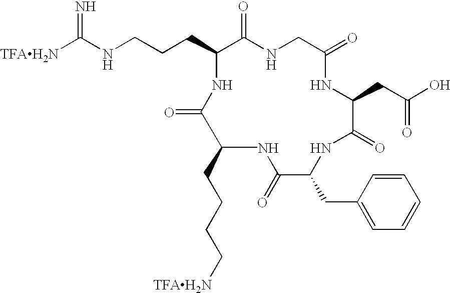

Cyclo{Arg(Tos)-Gly-Asp(OBzl)-D-Phe-Lys(Cbz)} (0.200 g, 0.204 mmol) was dissolved in trifluoracetic acid (0.6 mL) and cooled to −10° C. Trifluoromethanesulfonic acid (0.5 mL) was added dropwise, maintaining the temperature at −10° C. Anisole (0.1 mL) was added and the reaction was stirred at −10° C. for 3 h. Diethyl ether was added, the reaction was cooled to −50° C., and stirred for 1 h. The crude product was filtered, washed with diethyl ether, dried under high vacuum and purified by Preparative HPLC Method 1, to give 121.1 mg (71%) of the desired product as a lyophilized solid. HRMS: Calcd. for C₂₇H₄₁N₉O₇+H, 604.3207. Found, 604.3206. Analytical HPLC, Method 1B, Rₜ=11.197 min, Purity=100%.

Part C. Preparation of Boc-Glu(OSu)-OSu

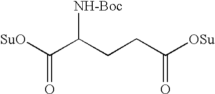

To a solution of Boc-Glu-OH (8.0 g, 32.25 mmol), N-hydroxysuccinimide (8.94 g, 77.64 mmol), and DMF (120 mL) was added 1-(3-dimethylaminopropyl)-3-ethylcarbodimide (14.88 g, 77.64 mmol). The reaction mixture was stirred at room temperature for 48 h. The mixture was concentrated under high vacuum and the residue was brought up in 0.1 N HCl and extracted with ethyl acetate (3×). The combined organic extracts were washed with water, saturated sodium bicarbonate and then saturated sodium chloride, dried over MgSO₄, and filtered. The filtrate was concentrated in vacuo and purified via reverse-phase HPLC (Vydac C18 column, 18 to 90% acetonitrile gradient containing 0.1% TFA, Rₜ=9.413 min) to afford 8.5 g (60%) of the desired product as a white powder. ¹H NMR (CDCl₃): 2.98-2.70 (m, 11H), 2.65-2.25 (m, 2H), 1.55-1.40 (s, 9H); ESMS: Calculated for C₁₈H₂₃N₃O₁₀, 441.1383. Found 459.2 [M+NH₄]+1.

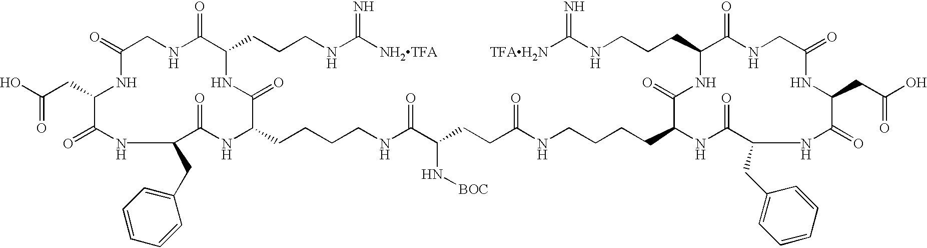

To a solution of cyclo(Lys-Arg-Gly-Asp-D-Phe) (0.050 g, 0.0601 mmol) in dimethylformamide (2 mL) was added triethylamine (25.1 μL, 0.183 mmol). After stirring for 5 minutes Boc-Glu(OSu)-OSu (0.0133 g, 0.0301 mmol) was added. The reaction mixture was stirred under N₂ for 20 h, then concentrated to an oil under high vacuum and triturated with ethyl acetate. The product thus obtained was filtered, washed with ethyl acetate, and dried under high vacuum to give 43.7 mg (44%) of the desired product. ESMS: Calcd. for C₆₄H₉₅N₁₉O₁₈, 1417.71. Found, 1418.8 [M+H]+1. Analytical HPLC, Method 1B, Rₜ=19.524 min, Purity=73%.

Part E. Preparation of Glu(cyclo{Lys-Arg-Gly-Asp-D-Phe})-cyclo{Lys-Arg-Gly-Asp-D-Phe} TFA salt.

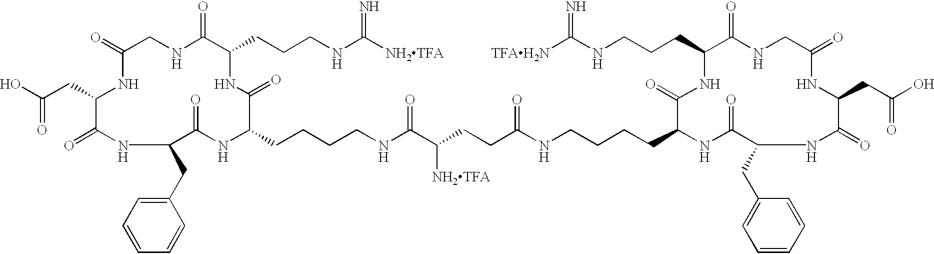

To a solution of Boc-Glu(cyclo{Lys-Arg-Gly-Asp-D-Phe})-cyclo{Lys-Arg-Gly-Asp-D-Phe}(0.040 g, 0.0243 mmol) in methylene chloride (1 mL) was added trifluoroacetic acid (1 mL). The reaction mixture was stirred for 2 h, concentrated to an oil under high vacuum and triturated with diethyl ether. The product was filtered, washed with diethyl ether, and dried under high vacuum to give 39.9 mg (100%) of the desired product. ESMS: Calcd. for C₅₉H₈₇N₁₉O₁₆, 1317.66. Found, 1318.9 [M+H]+1. Analytical HPLC, Method 1B, Rₜ=15.410 min, Purity=73%.

Part F. Preparation of 2-(1,4,7,10-teteaaza-4,7,10-tris(t-butoxycarbonylmethyl)-1-cyclododecyl)acetyl-Glu(cyclo{Lys-Arg-Gly-Asp-D-Phe})-cyclo{Lys-Arg-Gly-Asp-D-Phe}

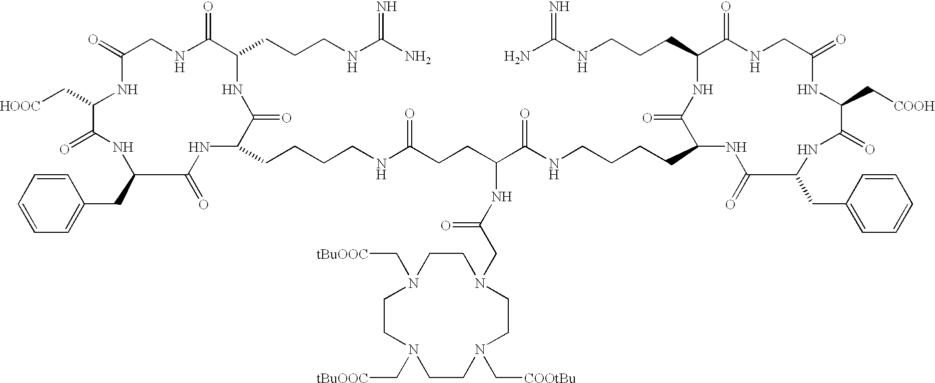

To a solution of tris(t-butyl)-1,4,7,10-tetra-azacyclododecane-1,4,7,10-tetraacetic acid (28 mg, 0.049 mmol, obtained from Macrocyclics, Inc., Richardson, TX) and Hunig's base (14 μL) in DMF (2 mL) was added HBTU (17 mg, 0.0456 mmol) and the mixture stirred for 5 min. To this was added a solution of Glu(cyclo{Lys-Arg-Gly-Asp-D-Phe})-cyclo{Lys-Arg-Gly-Asp-D-Phe} (54.1 mg, 0.0326 mmol) in DMF (1 mL) and the reaction mixture allowed to stir under nitrogen at room temperature for 4 h. The solvent was removed in vacuo and the residue purified by preparative RP-HPLC to give the product as a lyophilized solid (18.3 mg) (TFA salt). ESMS: Calcd. for C₈₇H₁₃₇N₂₃O₂₃, 1872.0. Found, 937.2 [M+2H]+2. Analytical HPLC, Method 1B, Rₜ=19.98 min, Purity=99%.

Part G. Preparation of 2-(1,4,7,10-tetraaza-4,7,10-tris(carboxymethyl)-1-cyclododecyl-Glu(cyclo{Lys-Arg-Gly-Asp-D-Phe})-cyclo{Lys-Arg-Gly-Asp-D-Phe}

A solution of 2-(1,4,7,10-tetraaza-4,7,10-tris(t-butoxycarbonylmethyl)-1-cyclododecyl)acetyl-Glu(cyclo{Lys-Arg-Gly-Asp-D-Phe})-cyclo{Lys-Arg-Gly-Asp-D-Phe} (18.3 mg, 8.71 mmol) in TFA (3 mL) was stirred at room temperature under nitrogen for 5 h. The solution was concentrated in vacuo and the residue was purified by preparative RP-HPLC to give 8 mg (45%) of the desired product as the lyophilized solid (TFA salt). ESMS: Calcd. for C₇₅H₁₁₃N₂₃O₂₃, 1703.8. Found, 853.0 [M+2H]+2. Analytical HPLC, Method 1B, Rₜ=13.13 min, Purity=99%.

| HPLC Method 1 | |
|---|---|
| Instrument: | HP 1050 |
| Column: | Vydac C18 (4.6 × 250 mm) |
| Detector: | Diode array detector 220 nm/500 ref |
| Flow Rate: | 1.0 mL/min. |
| Column Temp: | 50_C |
| Sample Size: | 15 uL |
| Mobile Phase: | A: 0.1% TFA in water |
| | B: 0.1% TFA in ACN/Water (9:1) |

| Gradient A: | Time (min) | % A | % B |
|---|---|---|---|
| | 0 | 80 | 20 |
| | 20 | 0 | 100 |
| | 30 | 0 | 100 |
| | 31 | 80 | 20 |
| Gradient B: | Time (min) | % A | % B |
| | 0 | 98 | 2 |
| | 16 | 63.2 | 36.8 |
| | 18 | 0 | 100 |
| | 28 | 0 | 100 |
| | 30 | 98 | 2 |

Compound B

Part A—Ethyl 4-(3,5-Dimethylphenoxy)butanoate

Sodium metal (17.12 g, 0.744 mol) was added to anhydrous EtOH (359 mL) abd stirred until dissolved. 3,5-Dimethylphenol was added and the solution was stirred 15 min at ambient temperatures. Ethyl 4-bromoacetate (58.7 mL, 0.41 mol) was added and the solution was stirred at ambient temperatures under a nitrogen atmosphere for 28 h. The EtOH was removed under vacuum and the oily solid was partitioned between water (1 L) and EtOAc (500 mL). The acqeous was extracted with additional EtOAc (500 mL). The combined EtOAc extracts were washed consecutively with saturated NaHCO3 (300 mL) and saturated NaCl (300 mL), dried (MgSO₄), and concentrated to give an amber liquid. The liquid was vacuum fractional distilled through a 15 cm Vigreux column. The main fraction was collected from 91-117° C./6 mm Hg to gave the title comounnd as a colorless liquid (77.77 g, 89%). ¹H NMR (CDCl₃): 6.59 (s, 1H), 6.52 (s, 2H), 4.16 (q, J -7.16 Hz, 2H), 3.98 (t, J=6.14 Hz, 2H), 2.49 (t,J=7,34 Hz, 2H), 2.28 (s, 6H), 2.11-2.07 (m, 2H), 1.26 (t, J=7.16 Hz, 3H); Anal. calcd for C₁₄H₂₀O₃: C,71.16; H, 8.53. Found: C,71.35; H, 8.59.

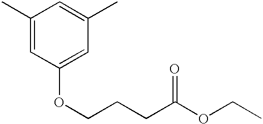

Part B—4-(3,5-Dimethylphenoxy)butanoic Acid

The product of part A, above (75.52 g, 0.320 mol) and KOH pellets (38.5 g, 0.584 mol) were dissolved in absolute EtOH (1.50 L) and heated at reflux for 3 h. The solution was concentrated to a colorless solid, which was taken up in water (2.0 L) and washed with ether (2×750 mL). The aqueous layer was adjusted to pH 1 with concd HCl (55 mL) and the resulting oily ppt was extracted into EtOAc (2×500 mL). The combined EtOAc extracts were washed consecutively with water (300 mL) and saturated NaCl, dried (MgSO₄), and concentrated to give a colorless solid (64.13 g). Recrystallization from hexanes (500 mL) gave the title compound as a colorless solid (59.51 g, 89%). MP: 66-68.5° C.; ¹H NMR (CDCl₃): 11.70 (bs, 1H), 6.59 (s, 1H), 6.52 (s, 2H), 3.99 (t, J=6.06 Hz, 2H), 2.57 (t, J=7.29 Hz, 2H), 2.28 (s, 6H), 2.12-2.08 (m, 2H); Anal. calcd for C₁₂H₁₁₆O₃: C, 69.21; H, 7.74. Found: C, 69.23; H, 7.40.

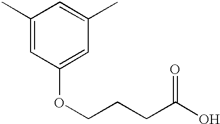

Part C—4-(4-(Chlorosulfonyl)-3,5-dimethylphenoxy)butanoic Acid

A solution of the product of Part B, above (20.8 g, 0.100 mol) in CHCl3 (100 mL) was cooled to 0° C. and treated with chlorosulfonic acid (36 mL, 0.54 mol) dropwise and with rapid stirring while keeping the temperature of the reaction at 0° C. The resulting gelatinous mixture was stirred an additional 10 min and poured onto an ice/water mixture (600 mL). The resulting solid ppt was collected by filtration, washed with water (3×75 mL), and dried under vacuum to give a colorless solid (12.52 g). MP: 114-115° C. (with decomp); ¹H NMR (CDCl₃): 13.84 (bs, 1H), 6.50 (s, 2H), 3.91 (t, J=6.48 Hz, 2H), 2.48 (s, 6H), 2.32 (t, J=7.32 Hz, 2H), 1.89-1.84 (m, 2H); IR (KBr cm⁻¹): 1705 (s), 1370

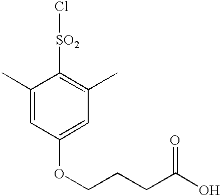

(s), 1175 (s); MS: m/e 305.1 [M-H].

Part D—4-(4-(((2-((tert-Butoxy)carbonylamino)-1-(methoxycarbonyl)ethyl)amino)sulfonyl)-3,5-dimethylphenoxy)butanoic Acid

A solution of N-b-Boc-L-a,b,-diaminopropionic acid methyl ester hydrochloride (568 mg, 2.10 mmol) and DIEA (0.73 mL, 4.2 mmol) in DCM (5 mL) was cooled to 0° C. and treated with a suspension of the product of Part C, above (656 mg, 2.10 mmol) in DCM (20 mL) in small portions over a 15 min period. The reaction was stirred at ambient temperatures under a nitrogen atmosphere for 18 h. The reaction was diluted with DCM (100 mL) and washed with water (3×75 mL). The organic phase was dried (MgSO₄), and concentrated to give crude product (698 mg), which was purified by preparative HPLC on a Vydac C-18 column (50×250 mm) using a 0.96%/min gradient of 18 to 58.5% ACN containing 0.1% TFA at a flow rate of 80 mL/min. The main product fraction eluting at 23.8 min was collected adjusted to pH 3, partially concentrated to remove ACN, and extracted with DCM (2×100 mL). The DCM extracts were dried (MgSO₄) and concentrated to give the title compound as a colorless solid (297 mg, 29%). ¹H NMR (CDCl₃): d 6.61 (s, 2H), 5.66 (d, J=7.2 Hz, 1H), 4.90 (s, 1H), 4.03 (bs, 2H), 3.86 (bs, 1H), 3.59 (s, 3H), 3.49 (bs, 2H), 2.62 (s, 6H), 2.58-2.51 (m, 2H), 2.18-2.07 (m, 2H), 1.41 (s, 9H); MS: m/e 489.4 [M+H]; High Resolution MS: Calcd for C₂₁H₃₃N₂O₉S [M+Na]: 511.1726. Found: 511.1747; Anal. calcd for C₂₁H₃₂N₂O₉S: C, 51.62; H, 6.61; N, 5.74, Found: C, 51.47; H, 6.27; N, 5.48.

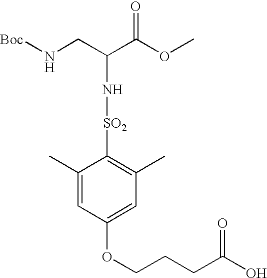

Part E—Preparation of Methyl (2S)-3-[(tert-Butoxy)carbonylamino]-2-[({2,6-dimethyl]-4-[3-(N-{2-[(phenylmethoxy)carbonylamino]ethyl}carbonyl)propoxy]-phenyl}sulfonyl)amino]propanoate

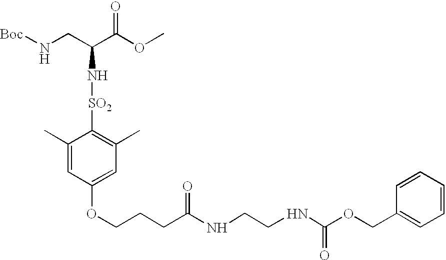

A solution of the product of Part D (369 mg, 0.756 mmol), DIEA (0.52 mL, 3.0 mmol), and HBTU (315 mg, 0.832 mmol) in anhydrous DMF (14 mL) was stirred at ambient temperatures under nitrogen for 5 min, and treated with benzyl N-(2-aminoethyl)carbamate hydrochloride (192 mg, 0.832 mmol), and stirred an additional 1 h. The DMF was removed under vacuum, and the oily residue was taken up in EtOAc (150 mL), washed consecutively with 0.1 N HCl (40 mL), water (40 mL), and saturated NaCl (40 mL), dried (MgSO₄), and concentrated to give a colorless viscous oil. Flash chromatography on a 3×16 cm silica gel column (EtOAc) gave the title compound as a colorless viscous oil (450 mg, 89.6%). ¹H NMR (CDCl₃): d 7.34-7.27 (m, 5H), 6.58 (s, 2H), 6.31 (bs, 1H), 5.86 (bs, 1H), 5.36 (bs, 1H), 5.14-5.03 (m, 3H), 3.96 (t, J=6.0 Hz, 2H), 3.88-3.83 (m, 1H), 3.56 (s, 3H), 3.47-3.25 (m, 6H), 2.59 (s, 6H), 2.31 (t, J=6.9 Hz, 2H), 2.05 (p, J=6.6 Hz, 2H), 1.39 (s, 9H); ¹³C NMR (CDCl₃): d 172.9, 170.5, 160.6, 157.3, 155.9, 141.8, 136.3, 128.5, 128.2, 128.0, 116.6, 79.9, 66.9, 55.5, 52.8, 43.1, 40.9, 40.3, 32.4, 28.2, 24.9, 23.3; MS: m/e 665.4 [M+H]; 687.3 [M+Na]; High Resolution MS: Calcd for C₃₁H₄₅N₄O₁₀S [M+H]: 665.2856. Found: 665.2883.

Part F—Preparation of Methyl (2S)-3-Amino-2-[({2,6-dimethyl-4-[3-(N-{2-[(phenylmethoxy)carbonylamino]ethyl}carbamoyl)propoxy]-phenyl}sulfonyl)amino]propanoate Trifluoroacetate Salt

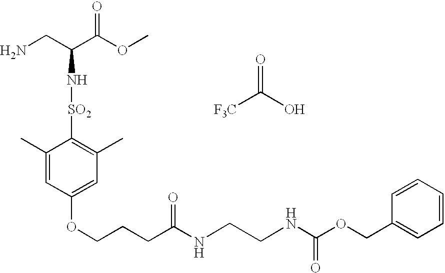

The product of Part E, above (420 mg, 0.632 mmol) was dissolved in 25/75 DCM/TFA (20 mL) and allowed to stand at ambient temperatures under nitrogen for 10 min. The solution was concentrated, and the resulting viscous oil was dissolved in 50% ACN and lyophilized to give the title compound as a colorless solid (437 mg, 102%). MS: m/e 565.3 [M+H].

Part G—Preparation of Methyl (2S)-2-[({2,6-Dimethyl-4-[3-(N-{2-[(phenylmethoxy)carbonylamino]ethyl}carbamoyl)-propoxy]phenyl}sulfonyl)amino]-3-{[1-methyl-4-oxo-7-({[1-(triphenylmethyl)imidazol-2-yl]amino}methyl)(3-hydroquinolyl)]carbonylamino}propanoate

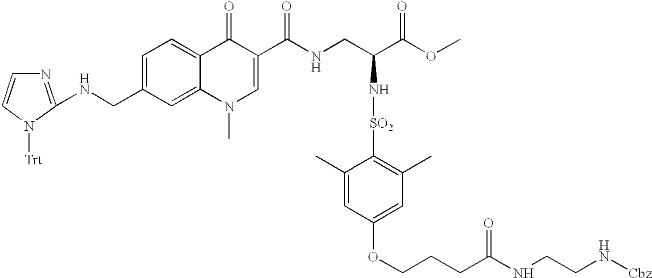

A solution of 1-methyl-4-oxo-7-(((1-(triphenylmethyl)imidazol-2-yl)amino)methyl)hydroquinoline-3-carboxylic acid (702 mg, 1.30 mmol), DIEA (0.678 mL, 3.90 mmol), and HBTU (542 mg, 1.43 mmol) in anhydrous DMF (60 mL) was stirred at ambient temperatures under nitrogen for 10 min, and treated with the product of Step F, above (881 mg, 1.30 mmol). After 75 min the DMF was removed under vacuum and the resulting oil was purified by HPLC on a Vydac C-18 column (50 x 250 mm) using a 1.24%/min gradient of 18 to 67.5% ACN containing 0.1% TFA at a flow rate of 80 mL/min. A peak eluting at 18.9 min was lyophilized to give unreacted 1-methyl-4-oxo-7-(((1-(triphenylmethyl)-imidazol-2-yl)amino) methyl)hydroquinoline-3-carboxylic acid (308 mg). The main product peak eluting at 23.7 min was lyophilized to give the title compound as a colorless solid (890 mg, 63.0%). ¹H NMR (CDCl₃/D₂O): d 8.50 (s, 1H), 8.18 (d, J=8.3 Hz, 1H), 7.70 (s, 11H), 7.51-7.25 (m, 15H), 7.25-7.12 (m, 5H), 6.97 (s, 1H), 6.58 (d, J=2.3 Hz, 1H), 6.34 (s, 2H), 6.32 (d, J=8.5 Hz, 1H), 5.09 (s, 2H), 4.65 (s, 2H), 4.29-4.23 (m, 1H), 3.88 (s, 3H), 3.80-3.50 (m, 7H), 3.41-3.28 (m, 4H), 2.61 (s, 6H), 2.26-2.11 (m, 2H), 1.92-1.76 (m, 2H); MS: m/e 1087.4 [M+H]; 845.3 [M+H-Tr]; High Resolution MS: Calcd for C₆₀H₆₃N₈0₁₀S [M+H]: 1087.4388. found: 1087.440.

Part H—Preparation of Methyl (2S)-2-{[(4-{3-[N-(2-Aminoethyyl)carbamoyl]propoxy}-2,6-dimethylphenyl)sulfonyl]amino}-3-{[1-methyl-4-oxo-7-({[1-triphenylmethyl)imidazol-2-yl]amino}methyl)(3-hydroquinolyl)]carbonylamino}propanoate

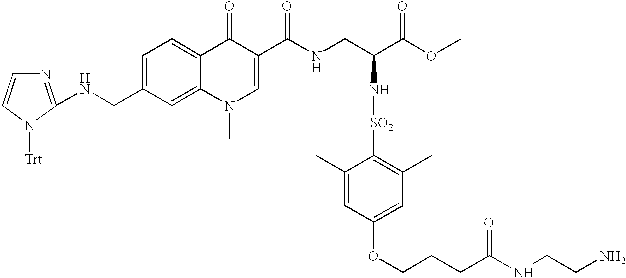

Hydrogenolysis of the product of Part G, above (468 mg, 0.431 mmol) was accomplished in MeOH (100 mL) over 10% Pd/C (95 mg) at 60 psi for 1 h. The catalyst was removed by filtration through Celite and the filtrate was concentrated to give the title compound as a pale amber oil (405 mg, 98.7%). MS: m/e 953.3 [M+H], 711.3 [M+H-Trityl].

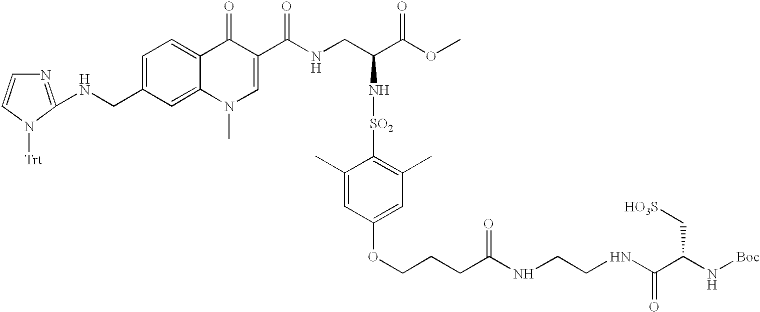

A solution of the product of Part H, above (405 mg, 0.425 mmol), the p-nitrophenyl ester of Boc-L-cysteic acid (425 mg, 1.03 mmol), and DIEA (0.435 mL, 2.55 mmol) in anhydrous DMF (20 mL) was stirred at ambient temperatures under nitrogen for 3 h. The DMF was removed under vacuum and the resulting oil was purified by HPLC on a Vydac C-18 column (50×250 mm) using a 1.12%/min gradient of 9 to 54% ACN containing 0.1% TFA at a flow rate of 80 mL/min. The main product peak eluting at 37.3 min was lyophilized to give the title compound as a colorless solid (410 mg, 80.2%). MS: m/e 1204.4 [M+H], 962.3 [M+H-Trt].

Part J—Preparation of (2R)-N-{2-[4-(4-{[n ((1S)-1-(Methoxycarbonyl)-2-{[1-methyl-4-oxo-7-({[1-(triphenylmethyl)imidazol-2-yl]amino}methyl)(3-hydroquinolyl)]carbonylamino}ethyl)amino]sulfonyl}-3,5-dimethylphenoxy)butanoylamino]ethyl}-2-aminopropanesulfonic Acid

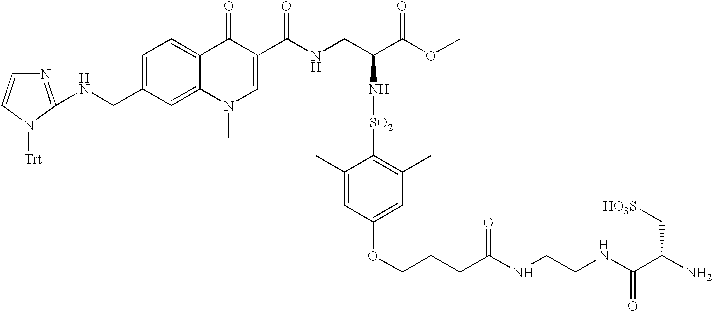

The product of Part I, above (410 mg, 0.341 mmol) was dissolved in 50/50 TFA/DCM (20 mL) and allowed to react at ambient temperatures for 10 min. The solution was concentrated and the resulting amber oil was dissolved in 50% ACN (50 mL) and lyophilized to give the title compound as a colorless solid (371 mg, 98.6%). MS: m/e 1104.4 [M+H], 862.3 [M+H-Trt]; High Resolution MS: Calcd for C₅₅H₆₂N₉O₁₂S₂ [M+H]: 1104.3959. Found: 1104.393.

Part K—Preparation of 2-({[4-(3-{N-[2-((2R)-2-Amino-3-sulfopropyl)ethyl]carbamoyl}propoxy)-2,6-dimethylphenyl]-sulfonyl}amino)(2S)-3-{[1-methyl-4-oxo-7-({[1-(triphenylmethyl)imidazol-2-yl]amino}methyl)(3-hydroquinolyl)]carbonylamino}propanoic Acid

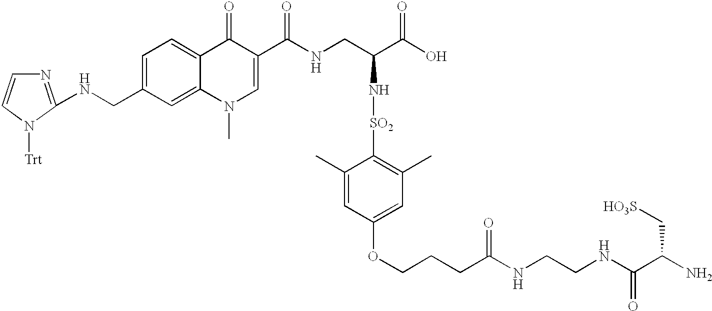

A mixture of the product of Part J (125 mg, 0.113 mmol), peroxide-free THF (3.8 mL), water (0.57 mL), and 3 N LiOH (0.38 mL, 1.13 mmol) was stirred at ambient temperatures under nitrogen for 1 h. The mixture was adjusted to pH 1 using 1 N HCl (0.70 mL) and concentrated to dryness under vacuum. The resulting solid was purified by HPLC on a Vydac C-18 column (50×250 mm) using a 0.90%/min gradient of 18 to 54% ACN containing 0.1% TFA at a flow rate of 80 mL/min. The main product peak eluting at 21.0 min was lyophilized to give the title compound as a colorless solid (96.0 mg, 77.9%). MS: m/e 1090.3 [M+H], 848.2 [M+H-Trt]; High Resolution MS: Calcd for C₅₄H₆₀N₉O₁₂S₂ [M+H]: 1090.3808. Found: 1090.381.

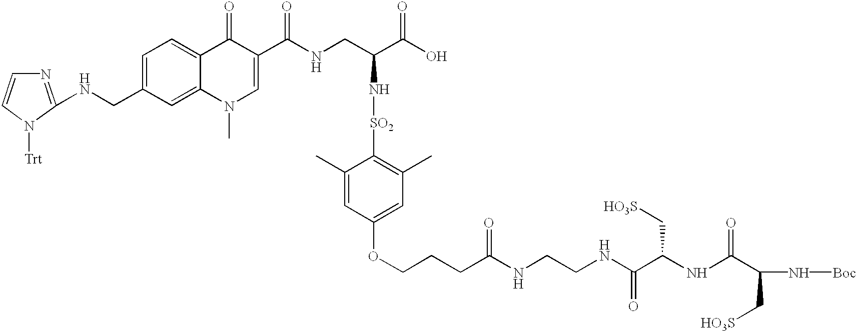

A solution of Boc-L-cysteic acid (37.0 mg, 0.128 mmol), DIEA (0.040 mL, 0.228 mmol), and PyBOP (53.0 mg, 0.102 mmol) in anhydrous DMF (1.0 mL) was stirred at ambient temperatures under nitrogen for 15 min, and added to a solution of the product of Part K, above (93.0 mg, 0.0854 mmol) and DIEA (0.045 mL, 0.256 mmol) in anhydrous DMF (3.0 mL). The resulting solution was stirred at ambient temperatures under nitrogen for 1.5 h and concentrated to a viscous amber oil. Purification by HPLC on a Vydac C-18 column (50×250 mm) using a 0.68%/min gradient of 18 to 45% ACN containing 0.1% TFA at a flow rate of 80 mL/min. The main product peak eluting at 36.4 min was lyophilized to give the title compound as a colorless solid (94.0 mg, 82.1%). MS: m/e 1341.2 [M+H], 1099.1 [M+H-Trt], 999.1 [M+H-Trt-Boc].

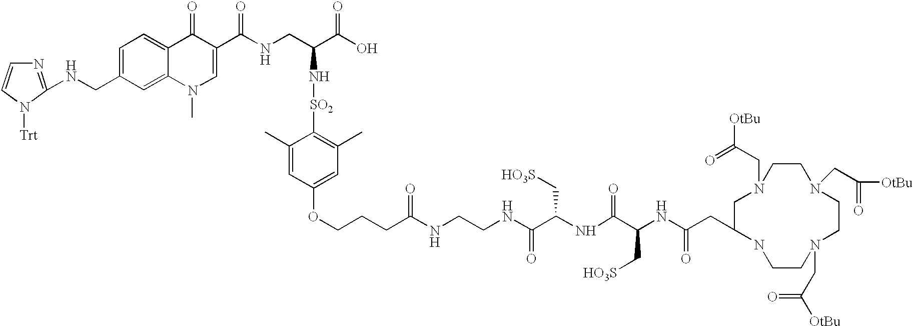

A solution of the product of Part L, above (90.0 mg, 0.0672 mmol) in 50/50 TFA/DCM (10.0 mL) was allowed to react at ambient temperatures under nitrogen for 10 min and concentrated under vacuum to give the intermediate amine as an amber oil. MS: m/e 1241.3 [M+H], 999.3 [M+H-Trt]; High Resolution MS: Calcd for C₅₇H₆₅N₁₀O₁₆S₃ [M+H]: 1241.3742. Found: 1241.375.

A solution of 2-(1,4,7,10-tetraaza-4,7,10-tris(((tert-butyl)-oxycarbonyl)methyl)cyclododecyl)acetic acid (123 mg, 0.134 mmol) (as described in DM-7003), DIEA (0.092 mL, 0.538 mmol), and PyBOP (52.4 mg, 0.101 mmol) in anhydrous DMF (1.5 mL) was stirred under nitrogen at ambient temperatures for 15 min, and added to a solution of the free amine produced above (90.0 mg, 0.0672 mmol) and DIEA (0.046 mL, 0.269 mmol) in anhydrous DMF (1.5 mL). The DMF was removed under vacuum after 1 h and the resulting amber oil was purified by HPLC on a Vydac C-18 column (50×250 mm) using a 0.288%/min gradient of 30.6 to 45% ACN containing 0.1% TFA at a flow rate of 80 mL/min. The main product peak eluting at 25.8 min was lyophilized to give the title compound as a colorless solid (92.0 mg, 76.3%). MS: m/e 1795.6 [M+H], 1553.5 [M+H-Trt]; High Resolution MS: Calcd for C₈₅H₁₁₅N₁₄O₂₃S₃ [M+H]: 1795.7422. Found: 1795.744.

Part N—Preparation of 2-[({4-[3-(N-{2-[(2R)-2-((2R)-3-Sulfo-2-{2-[1,4,7,10-tetraaza-4,7,10-tris(carboxymethyl)cyclododecyl]acetylamino}propyl)-3-sulfopropyl]ethyl}carbamoyl)propoxy]-2,6-dimethylphenyl}sulfonyl)amino](2S)-3-({7-[(imidazol-2-ylamino)methyl]-1-methyl-4-oxo(3-hydroquinolyl)}carbonylamino)propanoic Acid Trifluoroacetate Salt

A solution of the product of Part M, above (89.0 mg, 0.0496 mmol) in 97/3 TFA/Et3SiH (10.0 mL) was heated at 70° C. under nitrogen for 30 min and concentrated under vacuum. The resulting oily solid was purified by HPLC on a Vydac C-18 column (50×250 mm) using a 0.45%/min gradient of 4.5 to 22.5% ACN containing 0.1% TFA at a flow rate of 80 mL/min. The main product peak eluting at 19.5 min was lyophilized to give steochemically pure title compound as a colorless fluffy solid (65.0 mg, 87.5%). MS: m/e 1385.4 [M+H].

Compound C

Synthesis of DOTA/2-{[(4-{3-[N-(2-{(2R)-2-[(2R)-2-(4-{N-[(1R)-1-(N-{(1R)-1-[N-(2-{4-[4-({[(1S)-1-Carboxy-2-({7-[(imidazol-2-ylamino)methyl]-1-methyl-4-oxo(3-hydroquinolyl)}carbonylamino)1-carboxyethyl]amino}sulfonyl)-3,5-dimethylphenoxy]butanoylamino}ethyl)carbamoyl]-2-sulfoethyl}carbamoyl)-2-sulfoethyl]carbamoyl}(2S)-2-aminobutanoylamino)-3-sulfopropyl]-3-sulfopropyl}ethyl)carbamoyl]propoxy}-2,6-dimethylphenyl)dulfonyl]-amino}(2S)-3-({7-(imidazol-2-ylamino)methyl]-1-methyl-4-oxo(3-hydroquinolyl)}carbonylamino)propanoic Acid Conjugate

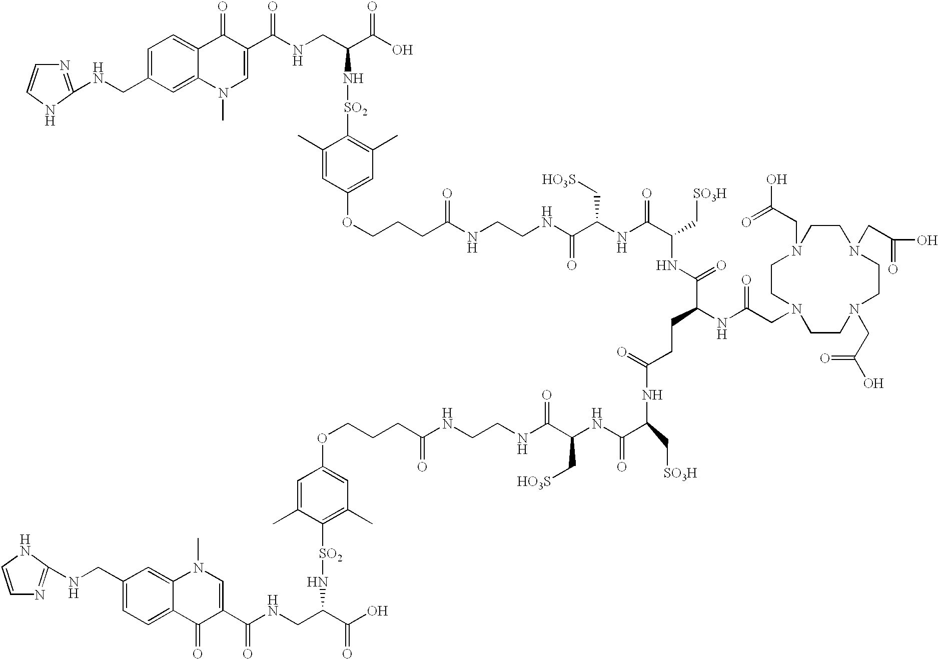

Part A—Preparation of Di-2,3,5,6-tetrafluorophenyl (2S)-2-[(tert-Butoxy)carbonylamino]pentane-1,5-dioate

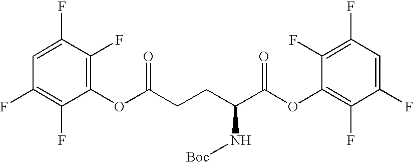

To a solution of Boc-L-Glu-OH (28.9 g, 117 mmol) in DMF (500 mL) at ambient temperatures and under nitrogen, was added a solution of 2,3,5,6-tetrafluorophenol (48.2 g, 290 mmol) in DMF (50 mL). After stirring for 10 min, EDC (55.6 g, 290 mmol) was added and the mixture was stirred for 96 h. The volatiles were removed under vacuum and the residue was triturated with 0.1 N HCl (750 mL). To this mixture was added EtOAc (600 mL) and the layers were separated. The aqueous layer was extracted with EtOAc (3×500 mL), and all EtOAc extracts were combined, washed consecutively with water (300 mL) and saturated NaCl (300 mL), dried (MgSO₃), and concentrated to give a tan solid (62 g). The tan solid was washed with ACN to give the title compound (45.5 g, 73.0%) in purified form. MS: m/e 566.0 [M+Na].

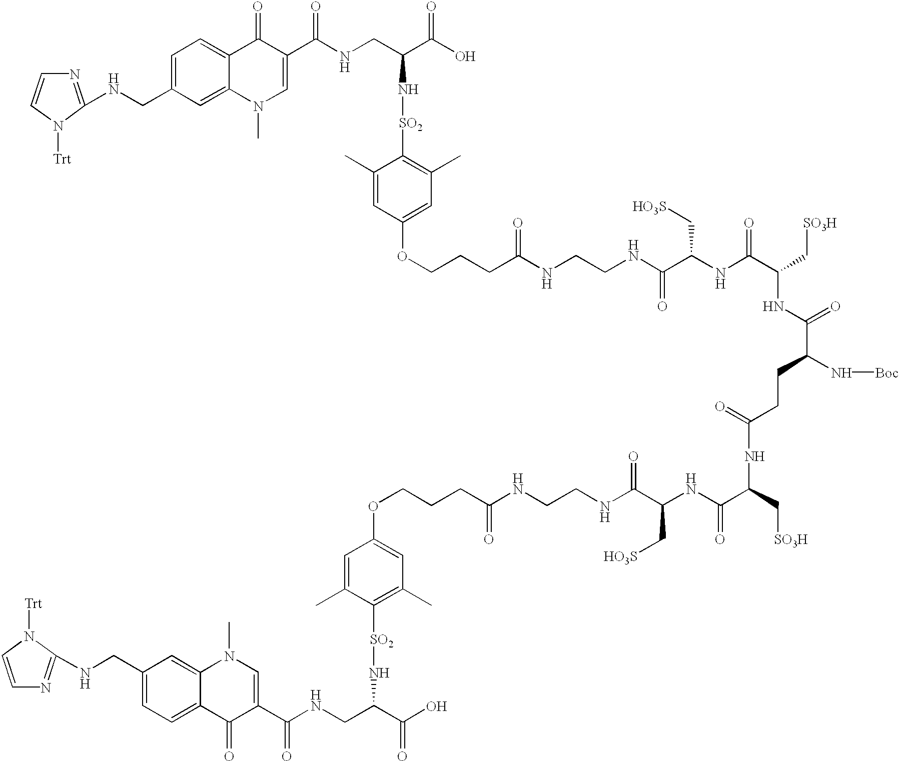

A solution of the product of the first half of the synthesis of Compound B, Part L (136 mg, 0.110 mmol), DIEA (0.076 mL, 0.44 mmol), and the product of Part A (26.2 mg, 0.050 mmol) in anhydrous DMF (3.0 mL) was stirred at ambient temperatures under nitrogen for 7 h. The DMF was removed under vacuum and the viscous amber oil was purified by HPLC on a Vydac C-18 column (50×250 mm) using a 0.45%/min gradient of 27 to 45% ACN, followed by a 0.72% gradient of 45-63% ACN containing 0.1% TFA at a flow rate of 80 mL/min. The main product peak eluting at 75.2 min was lyophilized to give the title compound as a colorless solid (129 mg, 47.9%). MS: m/e 1347.3 [M+2H].

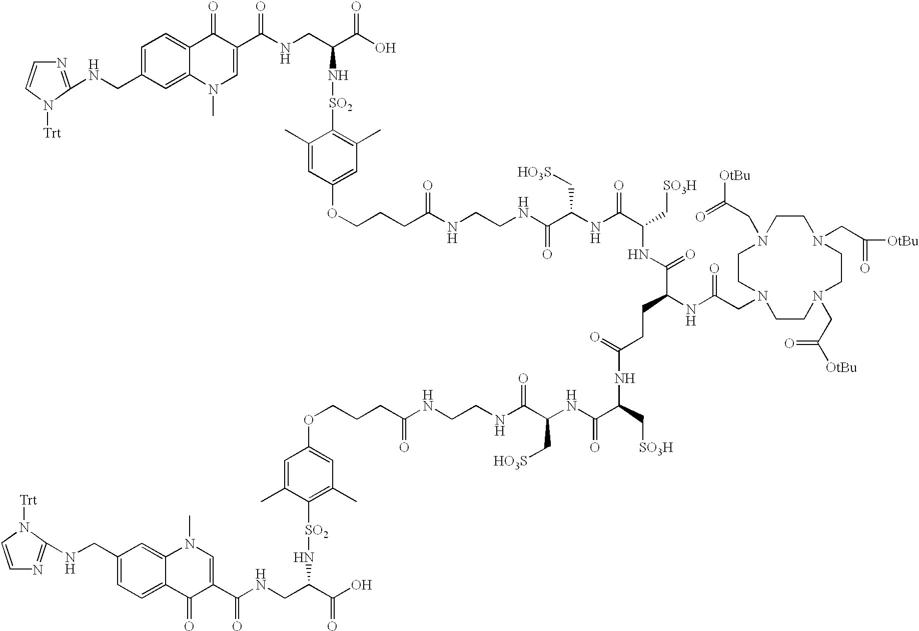

The product of Part B, above (34.0 mg, 0.0126 mmol) was dissolved in 50/50 TFA/DCM (12 mL) and allowed to react at ambient temperatures under nitrogen for 10 min. The solution was concentrated and the resulting amber oil was dried under vacuum.

A solution of 2-(1,4,7,10-tetraaza-4,7,10-tris(((tert-butyl)-oxycarbonyl)methyl)cyclododecyl)acetic acid (23.1 mg, 0.0253 mmol), DIEA (0.020 mL, 0.115 mmol), and PyBOP (9.8 mg, 0.019 mmol) in anhydrous DMF (2.0 mL) was stirred under nitrogen at ambient temperatures for 15 min, and added to a solution of the product from the deprotection reaction, above and DIEA (0.020 mL, 0.115 mmol) in anhydrous DMF (2.0 mL). The DMF was removed under vacuum after 2 h, and the resulting residue was purified by HPLC on a Vydac C-18 column (50×250 mm) using a 0.45%/min gradient of 27 to 49.5% ACN containing 0.1% TFA at a flow rate of 80 mL/min. The main product peak eluting at 43.8 min was lyophilized to give the title compound as a colorless solid (16.0 mg, 40.4%). MS: m/e 1574.8 [M+2H], 1453.7 [M+2H-Trt], 1332.2 [M+2H-2Trt].

Part D—Preparation of DOTA/2-{[(4-{3-[N-(2-{(2R)-2-[(2R)-2-(4-{N-[(1R)-1-(N-{(1R)-1-[N-(2-{4-[4-({[(1S)-1-Carboxy-2-({7-[(imidazol-2-ylamino)methyl]-1-methyl-4-oxo(3-hydroquinolyl)}carbonylamino)-1-carboxyethyl]amino}-sulfonyl)-3,5-dimethylphenoxy]butanoylamino}ethyl)carbamoyl]-2-sulfoethyl}carbamoyl)-2-sulfoethyl]carbamoyl}(2S)-2-aminobutanoylamino)-3-sulfopropyl]-3-sulfopropyl}ethyl)carbamoyl]propoxy}-2,6-dimethylphenyl)sulfonyl]amino}(2S)-3-({7-[(imidazol-2-ylamino)methyl]-1-methyl-4-oxo(3-hydroquinolyl)}carbonylamino)propanoic Acid Conjugate

The product of Part C, above (14.0 mg, 0.00445 mmol) was dissolved in 95/5 TFA/Et3SiH (8.0 mL) and heated at 70° C. under nitrogen for 1 h. The solution was concentrated under vacuum and the resulting yellow solid was purified by HPLC on a Vydac C-18 column (22×250 mm) using a 0.9%/min gradient of 0 to 27% ACN containing 0.1% TFA at a flow rate of 20 mL/min. The main product peak eluting at 24.5 min was lyophilized to give the title compound as a colorless solid (8.2 mg, 73.9%). MS: m/e 1247.7 [M+2H].

The radioisotope-chelator-optional linker-biomolecule complexes are designated by the isotope-compound letter, e.g. ⁹⁰Y-A is the ⁹⁰Y complex of 2-(1,4,7,10-tetraaza-4,7,10-tris(carboxymethyl)-1-cyclododecyl)acetyl-Glu(cyclo{Lys-Arg-Gly-Asp-D-Phe})-cyclo{Lys-Arg-Gly-Asp-D-Phe}.

### Example 1

### Preparation and Solution Stability of ⁹⁰Y-A Using Sodium Gentisate (GA, 10 mCi/mg) as the Stabilizer.

To a lead-shielded clean 5 mL vial containing 100 μg of COMPOUND A and 2 mg of sodium gentisate (GA) dissolved in 0.5 mL of 0.5 M ammonium acetate buffer (pH=7.5) was added 20 μL of ⁹⁰YCl₃ solution (˜20 mCi) in 0.05 N HCl. The reaction mixture was heated at 100° C. for 10 min. A sample of the resulting solution was first diluted 20-fold with 2 mM DTPA solution (pH=5), and was then analyzed by HPLC (Method 1, injection volume=1-2 μL) and ITLC. ITLC showed no [⁹⁰Y]colloid and [⁹⁰Y]acetate impurities. The resulting solution was kept at room temperature for 6 days. Samples were analyzed at t=24, 48, 120, and 144 h. The RCP data are listed in Table 1.

### Example 2

Preparation and Solution Stability of ⁹⁰Y-A Using 3,4,5-Trihydroxybenzaldehyde (3,4,5-THBAD, 10 mCi/mg) as the Stabilizer.

To a lead-shielded clean 5 mL vial containing 100 μg of COMPOUND A and 2 mg of 3,4,5-trihydroxy-benzaldehyde (3,4,5-THBAD) dissolved in 0.5 mL of 0.5 M ammonium acetate buffer (pH=7.5) was added 20 pL of ⁹⁰YCl₃ solution (˜20 mCi) in 0.05 N HCl. The reaction mixture was heated at 100° C. for 10 min. A sample of the resulting solution was first diluted 20-fold with 2 mM DTPA solution (pH=5), and was then analyzed by HPLC (Method 1, injection volume=1-2 μL) and ITLC. ITLC showed no [⁹⁰Y]colloid and [⁹⁰Y]acetate impurities. The resulting solution was kept at room temperature for 6 days. Samples were analyzed at t=24, 48, 120, and 144 h. RCP data are listed in Table 1.

### Example 3

Preparation and Solution Stability of ⁹⁰Y-A Using 3,4,5-Trihydroxybenzoic Acid (3,4,5-THBA, 10 mCi/mg) as the Stabilizer.

To a lead-shielded clean 5 mL vial containing 100 μg of COMPOUND A and 2 mg of 3,4,5-trihydroxybenzoic acid (3,4,5-THBA) dissolved in 0.5 mL of 0.5 M ammonium acetate buffer (pH=7.5) was added 20 pL of ⁹⁰YCl₃ solution (˜20 mCi) in 0.05 N HCl. The reaction mixture was heated at 100° C. for 10 min. A sample of the resulting solution was first diluted 20-fold with 2 mM DTPA solution (pH=5), and was then analyzed by HPLC (Method 1, injection volume=1-2 μL) and ITLC. ITLC showed no [⁹⁰Y]colloid and [⁹⁰Y]acetate impurities. The resulting solution was kept at room temperature for 6 days. Samples were analyzed at t=24, 48, 120, and 144 h. RCP data are listed in Table 1.

TABLE 1

RCP data for ⁹⁰Y-A prepared using 2 mg of sodium gentisate
(GA), 3,4,5-trihydroxybenzaldehyde (3,4,5-THBAD), and 3,4,5-
trihydroxybenzoic acid (3 ,4,5-THBA) as stabilizers.

| | RCP (%) | RCP (%) | RCP (%) | RCP (%) | RCP (%) |
|---|---|---|---|---|---|
| Stabilizer | t = 0 h | t = 24 h | t = 48 h | t = 120 h | t = 144 h |
| GA | 94.6 | 84.5 | 80.7 | 71.2 | 69.7 |
| 3,4,5- | 95.5 | 92.8 | 87.4 | 84.9 | 80.7 |
| THBAD | | | | | |
| 3,4,5- | 95.3 | 89.3 | 80.6 | 74.3 | 73.4 |
| THBA | | | | | |

The results demonstrated that 3,4,5-THBAD and 3,4,5-THBA are better stabilizers than GA for the ⁹⁰Y-labeled peptide vitronectin receptor antagonist at room temperature.

### Example 4

Preparation and Solution Stability of ⁹⁰Y-A Using Sodium Gentisate (GA, 2 mCi/mg) as the Stabilizer.

To a lead-shielded clean 5 mL vial containing 100 μg of COMPOUND A and 10 mg of sodium gentisate (GA) dissolved in 0.5 mL of 0.5 M ammonium acetate buffer (pH=7.5) was added 20 μL of ⁹⁰YCl₃ solution ( 20 mCi) in 0.05 N HCl. The reaction mixture was heated at 100° C. for 10 min. A sample of the resulting solution was first diluted 20-fold with 2 mM DTPA solution (pH=5), and was then analyzed by HPLC (Method 1, injection volume 1-2 μL) and ITLC. ITLC showed no [⁹⁰Y]colloid and [⁹⁰Y]acetate impurities. The resulting solution was kept at room temperature for 7 days. Samples were analyzed at t=24, 72, and 168 h. RCP data are listed in Table 2.

### Example 5

Preparation and Solution Stability of ⁹⁰Y-A Using 3,4,5-Trihydroxybenzaldehyde (3,4,5-THBAD, 2 mCi/mg) as the Stabilizer.

To a lead-shielded clean 5 mL vial containing 100 μg of COMPOUND A and 10 mg of 3,4,5-trihydroxy-benzaldehyde (3,4,5-THBAD) dissolved in 0.5 mL of 0.5 M ammonium acetate buffer (pH=7.5) was added 20 pL of ⁹⁰YCl₃ solution (˜20 mCi) in 0.05 N HCl. The reaction mixture was heated at 100° C. for 10 min. A sample of the resulting solution was first diluted 20-fold with 2 mM DTPA solution (pH=5), and was then analyzed by HPLC (Method 1, injection volume=1-2 μL) and ITLC. ITLC showed no [⁹⁰Y]colloid and [⁹⁰Y]acetate impurities. The resulting solution was kept at room temperature for 7 days. Samples were analyzed at t=24, 72, and 168 h. RCP data are listed in Table 2.

### Example 6

Preparation and Solution Stability of ⁹⁰Y-A Using 3,4,5-Trihydroxybenzoic Acid (3,4,5-THBA 2 mCi/mg) as the Stabilizer.

To a lead-shielded clean 5 mL vial containing 100 μg of COMPOUND A and 10 mg of 3,4,5-trihydroxybenzoic acid (3,4,5-THBA) dissolved in 0.5 mL of 0.5 M ammonium acetate buffer (pH=7.5) was added 20 pL of ⁹⁰YCl₃ solution (˜20 mCi) in 0.05 N HCl. The reaction mixture was heated at 100° C. for 10 min. A sample of the resulting solution was first diluted 20-fold with 2 mM DTPA solution (pH=5), and was then analyzed by HPLC (Method 1, injection volume=1-2 μL) and ITLC. ITLC showed no [⁹⁰Y]colloid and [⁹⁰Y]acetate impurities. The resulting solution was kept at room temperature for 7 days. Samples were analyzed at t=24, 72, and 168 h. RCP data are listed in Table 2.

### Example 7

Preparation and Solution Stability of ⁹⁰Y-A Using 2,4-Disulfonatobenzeneamine (DSA 2 mCi/mg) as the Stabilizer.

To a lead-shielded clean 5 mL vial containing 100 μg of COMPOUND A and 10 mg of 3,4,5-trihydroxy-benzoic acid (3,4,5-THBA) dissolved in 0.5 mL of 0.5 M ammonium acetate buffer (pH=7.5) was added 20 pL of ⁹⁰YCl₃ solution (˜20 mCi) in 0.05 N HCl. The reaction mixture was heated at 100° C. for 10 min. A sample of the resulting solution was first diluted 20-fold with 2 mM DTPA solution (pH=5), and was then analyzed by HPLC (Method 1, injection volume=1-2 μL) and ITLC. ITLC showed no [⁹⁰Y]colloid and [⁹⁰Y]acetate impurities. The resulting solution was kept at room temperature for 7 days. Samples were analyzed at t=24, 72, and 168 h. RCP data are listed in Table 2.

TABLE 2

RCP data for ⁹⁰Y-A prepared using 10 mg of sodium gentisate
(GA), 3,4,5-trihydroxybenzaldehyde (3,4,5-THBAD), 3,4,5-
trihydroxybenzoic acid (3,4,5-THBA), and 2,4-
disulfonatobenzeneamine (DSA) as stabilizers.

| | RCP (%) | RCP (%) | RCP (%) | RCP (%) | RCP (%) |
|---|---|---|---|---|---|
| Stabilizer | t = 0 h | t = 24 h | t = 72 h | t = 148 h | change |
| GA | 96.3 | 92.6 | 83.8 | 77.3 | 19 |
| 3,4,5- | 88.1* | 82.3 | 77.3 | 68.4 | 19.7 |
| THBAD | | | | | |
| 3,4,5- | 93.3 | 92.4 | 88 | 79.5 | 13.8 |
| THBA | | | | | |
| DSA | 92.2 | 89.3 | 82.8 | 80.5 | 11.7 |

### Example 8

Solution Stability of ⁹⁰Y-A at −78° C. Using Sodium Gentisate (GA, 2 mCi/mg) as the Stabilizer.

⁹⁰Y-A was prepared using the procedure described in example 4. The resulting solution was the kept in a dry-ice box (−78° C.) for 3 days. Samples were analyzed at t=0, 24, 48, and 72 h. RCP data are listed in Table 3.

### Example 9

Solution Stability of ⁹⁰Y-A at −78° C. Using 3,4,5-Trihydroxybenzaldehyde (3,4,5-THBAD, 2 mCi/mg).

⁹⁰Y-A was prepared using the procedure described in example 4. The resulting solution was the kept in a dry-ice box (−78° C.) for 3 days. Samples were analyzed at t=0, 24, 48, and 72 h. RCP data are listed in Table 3.

### Example 10

Solution Stability of ⁹⁰Y-A at −78° C. Using 3,4,5-Trihydroxybenzoic Acid (3,4,5-THBA, 2 mCi/mg) as the Stabilizer.

⁹⁰Y-A was prepared using the procedure described in example 4. The resulting solution was the kept in a dry-ice box (−78° C.) for 3 days. Samples were analyzed at t=0, 24, 48, and 72 h. RCP data are listed in Table 3.

### Example 11

Solution Stability of ⁹⁰Y-A at −78° C. Using 2,4-Disulfonatobenzeneamine (DSA, 2 mCi/mg) as the Stabilizer.

⁹⁰Y-A was prepared using the procedure described in example 4. The resulting solution was the kept in a dry-ice box (−78° C.) for 3 days. Samples were analyzed at t=0, 24, 48, and 72 h. RCP data are listed in Table 3.

TABLE 3

RCP data for ⁹⁰Y-A prepared using 10 mg of sodium gentisate
(GA),3,4,5-trihydroxybenzaldehyde (3,4,5-THBAD), 3,4,5-
trihydroxybenzoic acid (3,4,5-THBA), and 2,4-
disulfonatobenzeneamine (DSA) as stabilizers.

| | RCP (%) | RCP (%) | RCP (%) | RCP (%) | RCP (%) |
|---|---|---|---|---|---|
| Stabilizer | t = 0 h | t = 24 h | t = 48 h | t = 72 h | change |
| GA | 96.4 | 96.7 | 95.4 | 94.6 | 1.8 |
| 3,4,5- | 88.2 | 86.4 | 87.3 | 87.4 | 0.8 |
| THBAD | | | | | |
| 3,4,5- | 93.3 | 92.4 | 93.5 | 93.5 | 0.2 |
| THBA | | | | | |
| DSA | 94.4 | 94.0 | 94.1 | 94.2 | 0.2 |

### Example 12

Preparation and Solution Stability of ⁹⁰Y-A with 3,4,5-trihydroxybenzoic Acid (3,4,5-THBA, 2 mCi/mg) as the Stabilizer.

To a lead-shielded clean 5 mL vial containing 100 μg of COMPOUND A and 2 mg of 3,4,5-trihydroxybenzoic acid (3,4,5-THBA) dissolved in 0.5 mL of 0.5 M ammonium acetate buffer (pH=8.0) was added 20 μL Of ⁹⁰YCl₃ solution (˜20 mCi) in 0.05 N HCl. The reaction mixture was heated at 100° C. for 5 min. After cooling to room temperature, 0.5 mL of 3,4,5-THBA solution (20 mg/mL) in 0.5 M ammonium acetate buffer (pH=8.0) was added to the solution above. A sample of the resulting mixture was then diluted 20-fold with GA solution (10 mg/mL, pH=7.4), and analyzed by HPLC (Method 1, injection volume=1 μL) and ITLC. ITLC showed no [⁹⁰Y]colloid and [⁹⁰Y]acetate impurities. The resulting mixture was kept at −78° C. for 3 days. Samples were analyzed at t=0, 1, 2, and 3 days. RCP data are listed in Table 4.

### Example 13

Preparation and Solution Stability of ⁹⁰Y-A with 3,4,5-trihydroxybenzoic Acid (3,4,5-THBA 2 mCi/mg and 2,3,4-trihydroxybenzoic Acid (2,3,4-THBA) as Stabilizers.

Synthesis of ⁹⁰Y-A was carried out according to the procedure described in example 12. After cooling to room temperature, 0.5 mL of 2,3,4-THBA solution (20 mg/mL) in 0.5 M ammonium acetate buffer (pH=8.0) was added to the solution containing ⁹⁰Y-A. The resulting mixture was kept at -78 oC for 3 days. Samples were analyzed at t=0, 1, 2, and 3 days. RCP data are listed in Table 4.

### Example 14

Preparation and Solution Stability of ⁹⁰Y-A with 3,4,5-trihydroxybenzoic Acid (3,4,5-THBA, 2 mCi/mg and 2,4-disulfonatobenzeneamibe (DSA, 5 mCi/mg) Stabilizers.

Synthesis of ⁹⁰Y-A was carried out according to the procedure described in example 12. After cooling to room temperature, 0.5 mL of DSA solution (20 mg/mL) in 0.5 M ammonium acetate buffer (pH=8.0) was added to the solution containing ⁹⁰Y-A. The resulting mixture was kept at −78° C. for 3 days. Samples were analyzed at t=0, 1, 2, and 3 days. RCP data are listed in Table 4.

### Example 15

Preparation and Solution Stability of ⁹⁰Y-A with 3,4,5-trihydroxybenzoic Acid (3,4,5-THBA, 2 mCi/mg and 3-hydroxy-4-aminobenzoic Acid (HABA, 5 mCi/mg) as Stabilizers.

Synthesis of ⁹⁰Y-A was carried out according to the procedure described in example 12. After cooling to room temperature, 0.5 mL of HABA solution (20 mg/mL) in 0.5 M ammonium acetate buffer (pH=8.0) was added to the solution containing ⁹⁰Y-A. The resulting mixture was kept at −78° C. for 3 days. Samples were analyzed at t=0, 1, 2, and 3 days. RCP data are listed in Table 4.

TABLE 4

RCP data for ⁹⁰Y-A prepared using 2 mg of 3,4,5- 3,4,5-
trihydroxybenzoic acid (3,4,5-THBA). The second stabilizers,
including 3,4,5-THBA, 2,3,4-THBA, DSA and 3-hydroxy-4-
aminobenzoic acid (HABA), were added after radiolabeling.

| | RCP (%) | RCP (%) | RCP (%) | RCP (%) | RCP (%) |
|---|---|---|---|---|---|
| Stabilizer | t = 0 h | t = 24 h | t = 48 h | t = 72 h | change |
| 3,4,5- | 97.5 | 98.4 | 98.3 | 96.6 | 0.9 |
| THBA | | | | | |
| 2,3,4- | 98.1 | 99.2 | 99.0 | 98.2 | 0 |
| THBA | | | | | |
| DSA | 98.5 | 98.9 | 96.7 | 96.7 | 1.8 |
| HABA | 98.5 | 98.3 | 98.7 | 98.1 | 0.4 |

### Example 16

Preparation and Solution Stability of ⁹⁰Y-A Using 200 mCi of ⁹⁰Y and 5 mL Total Volume.

To a lead-shielded clean 30 mL vial containing 1000 μg of COMPOUND A and 100 mg of sodium gentisate (GA) dissolved in 5 mL of 0.5 M ammonium acetate buffer (pH=7.5) was added ˜250 μL Of ⁹⁰YCl₃ solution (˜200 mCi) in 0.05 N HCl. The reaction mixture was heated at 100° C. for 15 min. A sample of the resulting solution was first diluted 20-fold with 2 mm DTPA solution (pH=5), and was then analyzed by HPLC (Method 1, injection volume=1-2 μL) and ITLC. ITLC showed no [⁹⁰Y]colloid and [⁹⁰Y] acetate impurities. The resulting solution was kept at room temperature for 7 days. Samples were analyzed at t=72, and 168 h. RCP data are listed in Table 5.

### Example 17

Preparation and Solution Stability of ⁹⁰Y-A Using 200 mCi of ⁹⁰Y and 20 mL Total Volume.

To a lead-shielded clean 30 mL vial containing 1000 μg of COMPOUND A and 100 mg of sodium gentisate (GA) dissolved in 5 mL of 0.5 M ammonium acetate buffer (pH=7.5) was added ˜250 μL of ⁹⁰YCl₃ solution (˜200 mCi) in 0.05 N HCl. The reaction mixture was heated at 100° C. for 15 min. A sample of the resulting solution was analyzed and ITLC. The resulting solution was diluted to 20 mL and was then kept at −78° C. for 7 days. For comparison purpose, the second vial was made using 1000 μg of COMPOUND A, 100 mg of GA in 5 mL of 0.5 M ammonium acetate buffer (pH=7.5), and 200 mCi of ⁹⁰YCl₃. After radiolabeling, the reaction mixture was kept at −78° C. for 7 days without further dilution. Samples were analyzed at t=72 and 168 h. RCP data are listed in Table 5.

TABLE 5
RCP data for ⁹⁰Y − A prepared at 200 mCi activity level.

| Activity | RCP (%) | RCP (%) | RCP (%) |
|---|---|---|---|
| Concentration | t = 0 h | t = 72 h | t = 144 h |
| 200 mCi/5 mL | 94.8 | 93.0 | 94.3 |
| 200 mCi/20 mL | 94.0 | 93.7 | 94.0 |

The results demonstrated that: (1) the ⁹⁰Y-labeled peptide vitronectin receptor antagonist (90Y-A) can be prepared in high yield (RCP>90%) at 200 mCi activity level, (2) 90Y-A could be stabilized using a combination of sodium gentisate and low temperature (−78° C.) storage, and (3) the total volume did not have a significant effect on the solution stability of the radiopharmaceutical composition.

### Example 18

Preparation and Solution Stability of ⁹⁰Y-B in the Presence of Air.

To a lead-shielded clean 5 mL vial containing 100 μg of COMPOUND B and 10 mg of sodium gentisate (GA) dissolved in 0.5 mL of 0.5 M ammonium acetate buffer (pH=8.0) was added ˜20 μL of ⁹⁰YCl₃ solution (˜20 mCi) in 0.05 N HCl. The reaction mixture was heated at 100° C. for 5 min. After cooling to room temperature, a sample of the resulting solution was first diluted 20-fold with a GA solution (10 mg/mL in saline), and was then analyzed by HPLC (Method 2, injection volume=1-2 μL) and ITLC. ITLC showed no [⁹⁰Y]colloid and [⁹⁰Y]acetate impurities. The resulting solution was kept in a dry-ice box (−78° C.) for 7 days. Samples were analyzed at t=72 and 168 h. RCP data are listed in Table 6.

### Example 19

Preparation and Solution Stability of ⁹⁰Y-B with Exclusion of Air.

The 0.5 M ammonium acetate buffer (pH 8.0) was first degassed under vacuum for 1-2 min. COMPOUND B and sodium gentisate (GA) were dissolved in the degassed buffer to give a concentration of 200 μg/mL for COMPOUND B and 20 mg/mL for GA. The resulting solution was immediately degassed under vacuum for another 1-2 min. To a clean sealed 5 mL vial was added 0.5 mL of the degassed 0.5 M ammonium acetate buffer (pH 8.0) containing 100 μg COMPOUND B and 10 mg GA. The solution was degassed again under vacuum. Upon addition of 20-30 μL of ⁹⁰YCl₃ solution (20±2 mCi) in 0.05 N HCl, the reaction mixture was heated at 100° C. for 5 min. After cooling to room temperature, a sample of the resulting solution was diluted 20-fold with saline containing gentisate (10 mg/mL), and was then analyzed by HPLC (Method 2, injection volume 1-2 μL) and ITLC. ITLC showed no [⁹⁰Y]colloid and [⁹⁰Y]acetate impurities. The resulting solution was then kept in a dry-ice box (−78° C.) for 4 days. Samples were analyzed at t=24, 48 and 72 h. RCP data are listed in Table 6.

TABLE 6

RCP data for ⁹⁰Y-B prepared using 10 mg of sodium gentisate
(GA) as the stabilizer.

Buffer
Condition

| for radio- | RCP (%) | RCP (%) | RCP (%) | RCP (%) | RCP (%) |
|---|---|---|---|---|---|
| labeling | t = 0 h | t = 24 h | t = 48 h | t = 72 h | change |
| Non- | 90.5 | 86.5 | 84.5 | 84.3 | 6.2 |
| degassed | | | | | |
| Degassed | 93.9 | 92.5 | — | 90.5 | 3.4 |

The results clearly demonstrated that: (1) exclusion of air is beneficial for high yield preparation of the ⁹⁰Y-labeled vitronectin receptor antagonist (⁹⁰Y-B); (2) the presence of air in the radiopharmaceutical composition also has significant impact on the solution stability of ⁹⁰Y-B even at −78° C., and (3) a second stabilizer is beneficial for further enhancement of the solution stability of ⁹⁰Y-B.

### Example 20

Preparation and Solution Stability of ⁹⁰Y-B Using Sodium Gentisate (GA, 10 mCi/mg) as the Stabilizer and Sodium Ascorbate (AA, 1 mCi/mg) as the Second Stabilizer.

The 0.5 M ammonium acetate buffer (pH 8.0) was first degassed under vacuum for 1-2 min. COMPOUND B and gentisic acid (sodium salt, GA) were dissolved in the degassed buffer to give a concentration of 100 μg/mL for COMPOUND B and 20 mg/mL for GA. The resulting solution was immediately degassed under vacuum for another 1-2 min. To a clean sealed 5 mL vial was added 1.0 mL of the degassed 0.5 M ammonium acetate buffer (pH 8.0) containing 100 μg COMPOUND B and 10 mg GA. The solution was degassed again under vacuum. Upon addition of ˜20 μL of ⁹⁰YCl₃ solution (˜20 mCi) in 0.05 N HCl, the reaction mixture was heated at 100° C. for 5 min. After cooling to room temperature, a sample of the resulting solution was diluted 20-fold with saline containing gentisate (10 mg/mL), and was then analyzed by HPLC (Method 2, injection volume=1-2 μL) and ITLC. ITLC showed no [⁹⁰Y]colloid and [⁹⁰Y]acetate impurities. To the solution above was added 0.5 mL of sodium ascorbate solution (40 mg/mL in 0.5 M ammonium acetate buffer, pH=7.4). The resulting mixture was then kept in a dry-ice box (−78° C.) for 7 days. Samples were analyzed at t=0, 3 and 7 days. RCP data are listed in Table 7.

### Example 21

Preparation and Solution Stability of ⁹⁰Y-B Using Sodium Gentisate (GA, 10 mCi/mg) and 2,3,4-trihydroxybenzoic Acid (2,3,4-THBA, 2 mCi/mg) as the Stabilizers.

The 0.5 M ammonium acetate buffer (pH 8.0) was first degassed under vacuum for 1-2 min. COMPOUND B and gentisic acid (sodium salt, GA) were dissolved in the degassed buffer to give a concentration of 100 μg/mL for COMPOUND B and 20 mg/mL for GA. The resulting solution was immediately degassed under vacuum for another 1-2 min. To a clean sealed 5 mL vial was added 1.0 mL of the degassed 0.5 M ammonium acetate buffer (pH 8.0) containing 100 μg COMPOUND B and 10 mg GA. The solution was degassed again under vacuum. Upon addition of ˜20 μL of ⁹⁰YCl₃ solution (˜20 mCi) in 0.05 N HCl, the reaction mixture was heated at 100° C. for 5 min. After cooling to room temperature, a sample of the resulting solution was diluted 20-fold with saline containing gentisate (10 mg/mL), and was then analyzed by HPLC (Method 2, injection volume=1-2 μL) and ITLC. ITLC showed no [⁹⁰Y]colloid and [⁹⁰Y]acetate impurities. To the solution above was added 0.5 mL of 2,3,4-trihydroxybenzoic acid solution (20 mg/mL in 0.5 M ammonium acetate buffer, pH=6.5). The resulting mixture was then kept in a dry-ice box (−78° C.) for 7 days. Samples were analyzed at t=0, 3 and 7 days. RCP data are listed in Table 7.

### Example 22

Preparation and Solution Stability of ⁹⁰Y-B Using Sodium Gentisate (GA, 10 mCi/mg) and Mono Potassium 2,4-Disulfonatobenzeneamine (DSA, 1 mCi/mg) as the Stabilizers.

The 0.5 M ammonium acetate buffer (pH 8.0) was first degassed under vacuum for 1-2 min. COMPOUND B and gentisic acid (sodium salt, GA) were dissolved in the degassed buffer to give a concentration of 100 μg/mL for COMPOUND B and 20 mg/mL for GA. The resulting solution was immediately degassed under vacuum for another 1-2 min. To a clean sealed 5 mL vial was added 1.0 mL of the degassed 0.5 M ammonium acetate buffer (pH 8.0) containing 100 μg COMPOUND B and 10 mg GA. The solution was degassed again under vacuum. Upon addition of ˜20 μL of ⁹⁰YCl₃ solution (−20 mCi) in 0.05 N HCl, the reaction mixture was heated at 100° C. for 5 min. After cooling to room temperature, a sample of the resulting solution was diluted 20-fold with saline containing gentisate (10 mg/mL), and was then analyzed by HPLC (Method 2, injection volume=1-2 μL) and ITLC. ITLC showed no [⁹⁰Y]colloid and [⁹⁰Y]acetate impurities. To the solution above was added 0.5 nL of DSA solution (40 mg/mL in 0.5 M ammonium acetate buffer, pH=6.5). The resulting mixture was then kept in a dry-ice box (−78° C.) for 7 days. Samples were analyzed at t=0, 3 and 7 days. RCP data are listed in Table 7.

### Example 23

Preparation and Solution Stability of ⁹⁰Y-B Using Sodium Gentisate (GA, 10 mCi/mg) and 3-Amino-4-hydroxybenzoic acid (HABA, 2 mCi/mg) as the Stabilizers.

The 0.5 M ammonium acetate buffer (pH 8.0) was first degassed under vacuum for 1-2 min. COMPOUND B and gentisic acid (sodium salt, GA) were dissolved in the degassed buffer to give a concentration of 100 μg/mL for COMPOUND B and 20 mg/mL for GA. The resulting solution was immediately degassed under vacuum for another 1-2 min. To a clean sealed 5 mL vial was added 1.0 mL of the degassed 0.5 M ammonium acetate buffer (pH 8.0) containing 100 μg COMPOUND B and 10 mg GA. The solution was degassed again under vacuum. Upon addition of 20 μL of ⁹⁰YCl₃ solution (˜20 mCi) in 0.05 N HCl, the reaction mixture was heated at 100° C. for 5 min. After cooling to room temperature, a sample of the resulting solution was diluted 20-fold with saline containing gentisate (10 mg/mL), and was then analyzed by HPLC (Method 2, injection volume=1-2 μL) and ITLC. ITLC showed no [⁹⁰Y]colloid and [⁹⁰Y]acetate impurities. To the solution above was added 0.5 mL of HASA solution (20 mg/mL in 0.5 M ammonium acetate buffer, pH=6.5). The resulting mixture was then kept in a dry-ice box (−78° C.) for 7 days. Samples were analyzed at t=0, 3 and 7 days. RCP data are listed in Table 7.

TABLE 7

RCP data for ⁹⁰Y-B prepared using 2 mg of sodium gentisate
(GA) as the stabilizer and sodium ascorbate (AA, 1 mCi/mg), 2,4-
disulfonatobenzene-amine (mono potassium salt, DSA, 1
mCi/mg), 2,3,4-trihydroxybenzoic acid (2,3,4-THBA, 2 mCi/mg),
and 3-Amino-4-hydroxybenzoic acid (HABA, 2 mCi/mg).

| | RCP (%) | RCP (%) | RCP (%) | RCP (%) |
|---|---|---|---|---|
| Second Stabilizer | t = 0 | t = 3 days | t = 7 days | change |
| No second stabilizer | 93.9 | 90.5 | 88 | 5.9 |
| 20 mg AA | 95.0 | 93.9 | 94.5 | 0.5 |
| 20 mg DSA | 94.4 | 94.8 | 94.5 | 0.1 |
| 10 mg 2,3,4-THBA | 94.2 | 93.6 | 93.9 | 0.3 |
| 10 mg HABA | 94.8 | 94.5 | 94.0 | 0.8 |

These results demonstrated that: (1) the ⁹⁰Y-labeled vitronectin receptor antagonist (⁹⁰Y-B) could be prepared with exclusion of air; and (2) ⁹⁰Y-B remained stable at −78° C. for at least 7 days by the post-labeling addition of a second stabilizer such as AA, DSA, 2,3,4-THBA, and HABA.

### Example 24

Preparation and Solution Stability of ¹⁷⁷Lu-C Using Sodium Gentisate (GA, 10 mCi/mg) and Sodium Ascorbate (AA, 1 mCi/mg) as the Stabilizers.

The 0.5 M ammonium acetate buffer (pH 8.0) was first degassed under vacuum for 1-2 min. COMPOUND C and gentisic acid (sodium salt, GA) were dissolved in the degassed buffer to give a concentration of 100 μg/mL for COMPOUND C and 2 mg/mL for GA. The resulting solution was immediately degassed under vacuum for another 1-2 min. To a clean sealed 5 mL vial was added 1.0 mL of the degassed 0.5 M ammonium acetate buffer (pH 8.0) containing 100 μg COMPOUND C and 2 mg GA. The solution was degassed again under vacuum. Upon addition of ˜20 μL of ¹⁷⁷LuCl₃ solution (˜20 mCi) in 0.05 N HCl, the reaction mixture was heated at 100° C. for 5 min. After cooling to room temperature, a sample of the resulting solution was analyzed by HPLC (Method 3, injection volume=5 μL) and ITLC. ITLC showed no [¹⁷⁷Lu]colloid and [¹⁷⁷Lu]acetate radioimpurities. To the solution above was added 0.5 mL of sodium ascorbate solution (40 mg/mL in 0.5 M ammonium acetate buffer, pH=7.4). The resulting mixture was then kept in a dry-ice box (−78° C.) for 7 days. Samples were analyzed at t=0, 3 and 7 days. RCP data are listed in Table 8.

### Example 25

Preparation and Solution Stability of ¹⁷⁷Lu-C Using Sodium Gentisate (GA, 10 mCi/mg) and 2,3,4-trihydroxybenzoic Acid (2,3,4-THBA, 2 mCi/mg) as the Stabilizers.

COMPOUND C and gentisic acid (sodium salt, GA) were dissolved in the degassed buffer to give a concentration of 100 μg/mL for COMPOUND C and 2 mg/mL for GA. The resulting solution was immediately degassed under vacuum for another 1-2 min. To a clean sealed 5 mL vial was added 1.0 μL of the degassed 0.5 M ammonium acetate buffer (pH 8.0) containing 100 μg COMPOUND C and 2 mg GA. The solution was degassed again under vacuum. Upon addition of ˜20 μL of ¹⁷⁷LuCl₃ solution (˜20 mCi) in 0.05 N HCl, the reaction mixture was heated at 100° C. for 5 min. After cooling to room temperature, a sample of the resulting solution was analyzed by HPLC (Method 3, injection volume=5 μL) and ITLC. ITLC showed no [¹⁷⁷Lu]colloid and [¹⁷⁷ Lu]acetate radioimpurities. To the solution above was added 0.5 mL of 2,3,4-THBA solution (20 mg/mL in 0.5 M ammonium acetate buffer, pH=7.4). The resulting mixture was then kept in a dry-ice box (−78° C.) for 7 days. Samples were analyzed at t=0, 3 and 7 days. RCP data are listed in Table 8.

### Example 26

Preparation and Solution Stabilitv of ¹⁷⁷Lu-C Using Sodium Gentisate (GA 10 mCi/mg) and Mono Potassium 2,4-Disulfonatobenzeneamine (DSA, 1 mCi/mg) as the Stabilizers.

COMPOUND C and gentisic acid (sodium salt, GA) were dissolved in the degassed buffer to give a concentration of 100 μg/mL for COMPOUND C and 2 mg/mL for GA. The resulting solution was immediately degassed under vacuum for another 1-2 min. To a clean sealed 5 mL vial was added 1.0 mL of the degassed 0.5 M ammonium acetate buffer (pH 8.0) containing 100 μg COMPOUND C and 2 mg GA. The solution was degassed again under vacuum. Upon addition of ˜20 μL of ¹⁷⁷LuCl₃ solution (˜20 mCi) in 0.05 N HCl, the reaction mixture was heated at 100° C. for 5 min. After cooling to room temperature, a sample of the resulting solution was analyzed by HPLC (Method 3, injection volume=5 μL) and ITLC. ITLC showed no [¹⁷⁷Lu]colloid and [¹⁷⁷ Lu]acetate radioimpurities. To the solution above was added 0.5 mL of DSA solution (20 mg/mL in 0.5 M ammonium acetate buffer, pH=7.4). The resulting mixture was then kept in a dry-ice box (−78° C.) for 7 days. Samples were analyzed at t=0, 3 and 7 days. RCP data are listed in Table 8.

### Example 27

Preparation and Solution Stability of ¹⁷⁷Lu-C Using Sodium Gentisate (GA, 10 mCi/mg) and 3,4,5-trihydroxybenzoic Acid (3,4,5-THBA, 2 mCi/mg) as the Stabilizers.

COMPOUND C and gentisic acid (sodium salt, GA) were dissolved in the degassed buffer to give a concentration of 100 μg/mL for COMPOUND C and 2 mg/mL for GA. The resulting solution was immediately degassed under vacuum for another 1-2 min. To a clean sealed 5 mL vial was added 1.0 mL of the degassed 0.5 M ammonium acetate buffer (pH 8.0) containing 100 μg COMPOUND C and 2 mg GA. The solution was degassed again under vacuum. Upon addition of ˜20 μL of ¹⁷⁷LuCl₃ solution (˜20 mCi) in 0.05 N HCl, the reaction mixture was heated at 100° C. for 5 min. After cooling to room temperature, a sample of the resulting solution was analyzed by HPLC (Method 3, injection volume=5 μL) and ITLC. ITLC showed no [¹⁷⁷Lu]colloid and [¹⁷⁷Lu]acetate radioimpurities. To the solution above was added 0.5 mL of 3,4,5-THBA solution (10 mg/mL in 0.5 M ammonium acetate buffer, pH=6.5). The resulting mixture was then kept in a dry-ice box (−78° C.) for 7 days. Samples were analyzed at t=0, 3 and 7 days. RCP data are listed in Table 8.

TABLE 8

RCP data for ¹⁷⁷prepared using 2 mg of sodium gentisate
(GA) as the stabilizer and sodium ascorbate (AA, 1 mCi/mg), 2,4-
disulfonatobenzene-amine (mono potassium salt, DSA, 1
mCi/mg), 2,3,4-trihydroxybenzoic acid (2,3,4-THBA, 2 mCi/mg),
and 3,4,5-trihydroxybenzoic acid (3,4,5-TUBA, 2 mCi/mg).

| Second | RCP (%) | RCP (%) | RCP (%) | RCP (%) | RCP (%) |
|---|---|---|---|---|---|
| Stabilizer | t = 0 | t = 1 day | t = 3 days | t = 7 days | change |
| 20 mg AA | 97.6 | 97.7 | 97.5 | 93.0 | 4.6 |
| 20 mg DSA | 97.4 | 97.4 | 95.9 | 93.0 | 4.4 |
| 10 mg 2,3,4- | 97.4 | 96.6 | 96.5 | 95.4 | 2.0 |
| THBA | | | | | |
| 10 mg 3,4,5- | 97.2 | 97.9 | 96.7 | 95.0 | 2.2 |
| THBA | | | | | |

These results demonstrated that: (1) the ¹⁷⁷Lu-labeled vitronectin receptor antagonist (¹⁷⁷Lu-C) could be prepared in high yield (RCP>95%) with exclusion of air, (2) ¹⁷⁷Lu-C remained stable at −78° C. for at least 7 days by the post-labeling addition of a second stabilizer such as AA, DSA, 2,3,4-THBA, and 3,4,5-THBA, and (3) 2,3,4-THBA and 3,4,5-THBA provided better stabilization for the ¹⁷⁷Lu-labeled vitronectin receptor antagonist (¹⁷⁷Lu-C) than AA and DSA.

### Example 28

Preparation and Solution Stability of ⁹⁰Y-C Using Sodium Gentisate (GA, 10 mCi/mg) as the Stabilizer.

0.5 M ammonium acetate buffer (pH 8.0) was first degassed under vacuum for 1-2 min. COMPOUND C and gentisic acid (sodium salt, GA) were dissolved in the degassed buffer to give a concentration of 100 μg/mL for COMPOUND C and 2 mg/mL for GA. The resulting solution was immediately degassed under vacuum for another 1-2 min. To a clean sealed 5 mL vial was added 1.0 mL of the degassed 0.5 M ammonium acetate buffer (pH 8.0) containing 100 μg COMPOUND B and 2 mg GA. The solution was degassed again under vacuum. Upon addition of ˜20 μL of ⁹⁰YCl₃ solution (˜20 mCi) in 0.05 N HCl, the reaction mixture was heated at 100° C. for 5 min. After cooling to room temperature, a sample of the resulting solution was analyzed by HPLC (Method 3, injection volume=5 μL) and ITLC. The RCP was 91.3%. ITLC showed no [90Y]colloid and [90Y]acetate radio-impurities.

All publications, patents, patent applications, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A pharmaceutical composition comprising:
(1.) a radiolabeled pharmaceutical agent of the formula (II)
RI-Ch-Lₙ-(BM)ₓ (II); and
(2.) an effective stabilizing amount of a compound of formula (I): 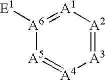
wherein
RI is ^{99m}Tc, ¹³¹I, ¹²⁵I, ¹²³I, ^{117m}Sn, ¹¹¹In, ⁹⁷Ru, ²⁰³Pb, ⁶⁷Ga, ⁶⁸Ga, ⁸⁹Zr, ⁹⁰Y, ¹⁷⁷Lu, ¹⁴⁹Pm, ¹⁵³Sm, ¹⁶⁶Ho, ¹³¹I, ³²P, ²¹¹At, ⁴⁷Sc, ¹⁰⁹Pd, ¹⁰⁵Rh, ¹⁸⁶Re, ¹⁸⁸Re, ⁶⁰Cu, ⁶²Cu, ⁶⁴Cu or ⁶⁷Cu;
Cₕ is a metal chelator or is a direct linkage;
Lₙ is a linking group or is a direct linkage;
each BM is independently a peptidomimetic or a non-peptide;
x is 1 to about 10;
E¹ is NH₂ or OH;
A¹, A², A³, A⁴ and A⁵ are each independently N, C(OH) or CR¹;
provided at least one of A¹, A², A³, A⁴ and A⁵ is not CH;
each R¹ is independently H, C(O)R², C(O)OR², NHC(═O)NHR², NHC(═S)NHR², OC(═O)R², OC(═O)OR², S(O)₂OR², C(O)NR³R⁴, C(O)NR³OR⁴, C(O)NR²NR³R⁴, NR³R⁴, NR³C(O)R⁴, PO(OR³)(OR⁴), S(O)₂NR³R⁴, S(O)₂NR²NR³R⁴, S(O)₂NR³OR⁴, C₁-C₁₀ alkyl substituted with 0-5 R⁵, C₃-C₁₀ cycloalkyl substituted with 0-5 R⁵, C₂-C₁₀ alkenyl substituted with 0-5 R⁵, or aryl substituted with 0-5 R⁵;
R², R³, and R⁴ are each independently H, C₁-C₆ alkyl, C₃-C₆ cycloalkyl, C₁-C₆ alkenyl, benzyl, or phenyl; or R³ and R⁴ together form C₃-C₁₀ cycloalkyl or C₃-C₁₀ cycloalkenyl, optionally interrupted with O, S, NH, S(═O), S(O)₂, P(═O)(OH), C(═O)NH, NHC(═O), NHC(═O)NH, or NHC(═S)NH; and
each R⁵ is independently H, NH₂, OH, CO₂H, C(═O)NH₂, C(═O)NHOH, C(═O)NHNH₂, NHC(═NH)NH₂, NHC(═O)NH₂, NHC(═S)NH₂, PO₃H₂, SO₃H, or S(O)₂NH₂;
or a pharmaceutically acceptable salt thereof.

2. The composition of claim 1 wherein
E¹ is OH;
A¹, A², A³, and A⁴ are each independently C(OH) or CR¹;
A⁵ is C(OH);
each R⁵ is independently H, C(O)R², C(O)OR², NHC(═O)NHR², NHC(═S)NHR², OC(═O)R², OC(═O)OR², S(O)₂OR², C(O)NR³R⁴, C(O)NR³OR⁴, C(O)NR²NR³R⁴, NR³R⁴, NR³C(O)R⁴, PO(OR³)(OR⁴), S(O)₂NR³R⁴, S(O)₂NR²NR³R⁴, S(O)₂NR³OR⁴, C₁-C₁₀ alkyl substituted with 0-3 R⁵, C₃-C₁₀ cycloalkyl substituted with 0-3 R⁵, C₂-C₁₀ alkenyl substituted with 0-3 R⁵, or aryl substituted with 0-5 R⁵;
R², R³, and R⁴ are each independently H, C₁-C₆ alkyl, C₃-C₆ cycloalkyl, benzyl, or phenyl; or R³ and R⁴ together form C₃-C₁₀ cycloalkyl optionally interrupted with O, S, NH, S(═O), S(O)₂, P(═O)(OH), C(═O)NH, NHC(═O), NHC(═O)NH, or NHC(═S)NH; and
each R⁵ is independently H, NH₂, OH, CO₂H, C(═O)NH₂, PO₃H₂, SO₃H, or S(O)₂NH₂.

3. The composition of claim 2 wherein,
A⁴ is C(OH); and
each R¹ is independently C(O)H, C(O)NH₂, C(O)NHNH₂, CO₂H, NHC(═O)NH₂, NHC(═S)NH₂, PO₃H₂, SO₃H, or S(O)₂NH₂.

4. The composition of claim 3 wherein the compound of formula (I) is: 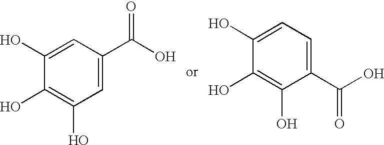
or a pharmaceutically acceptable salt thereof.

5. The composition of claim 1 wherein
E¹ is NH₂;
A¹, A², A³, and A⁴ are each independently C(OH) or CR¹;
A⁵ is C(OH);
each R¹ is independently H, C(O)R², C(O)OR², NHC(═O)NHR², NHC(═S)NHR², OC(═O)R², OC(═O)OR², S(O)₂OR², C(O)NR³R⁴, C(O)NR³OR⁴, C(O)NR²NR³R⁴, NR³R⁴, NR³C(O)R⁴, PO(OR³)(OR⁴), S(O)₂NR³R⁴, S(O)₂NR²NR³R⁴, S(O)₂NR³OR⁴, C₁-C₁₀ alkyl substituted with 0-3 R⁵, C₃-C₁₀ cycloalkyl substituted with 0-3 R⁵, C₂-C₁₀ alkenyl substituted with 0-3 R⁵, or aryl substituted with 0-5 R⁵;
R², R³, and R⁴ are each independently H, C₁-C₆ alkyl, C₃-C₆ cycloalkyl, benzyl, or phenyl; or R³ and R⁴ together form C₃-C₁₀ cycloalkyl optionally interrupted with O, S, NH, S(═O), S(O)₂, P(═O)(OH), C(═O)NH, NHC(═O), NHC(═O)NH, or NHC(═S)NH; and
each R⁵ is independently H, NH₂, OH, CO₂H, C(═O)NH₂, PO₃H₂, SO₃H, or S(O)₂NH₂.

6. The composition of claim 5 wherein
each R¹ is independently C(O)H, C(O)NH₂, C(O)NHNH₂, CO₂H, NHC(═O)NH₂, NHC(═S)NH₂, PO₃H₂, SO₃H, or S(O)₂NH₂.

7. The composition of claim 6 wherein compound of the formula (I) is a compound of the formula: 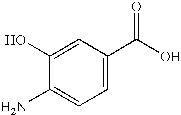
or a pharmaceutically acceptable salt thereof.

8. The composition of claim 1 wherein
A¹, A², A³, A⁴, and A⁵ are each independently N, C(OH) or CR¹; provided that A⁵ is not C(OH);
each R¹ is independently H, C(O)R , C(O)OR , NHC(═O)NHR², NHC(═S)NHR², OC(═O)R², OC(═O)OR², S(O)₂OR², C(O)NR³R⁴, C(O)NR³OR⁴,C(O)NR²NR³R⁴, NR³R⁴, NR³C(O)R⁴, PO(OR³)(OR⁴), S(O)₂NR³R⁴, S(O)₂NR²NR³R⁴, S(O)₂NR³OR⁴, C₁-C₁₀ alkyl substituted with 0-5 R⁵, C₃-C₁₀ cycloalkyl substituted with 0-5 R⁵, C₂-C₁₀ alkenyl substituted with 0-5 R⁵, or aryl substituted with 0-5 R⁵;
R², R³, and R⁴ are each independently H, C₁-C₆ alkyl, C₃-C₆ cycloalkyl, C₁-C₆ alkenyl, benzyl, or phenyl; or R³ and R⁴ together form C₃-C₁₀ cycloalkyl or C₃-C₁₀ cycloalkenyl optionally interrupted with O, S, NH, S(═O), S(O)₂, P(═O)(OH), C(═O)NH, NHC(═O), NHC(═O)NH, or NHC(═S)NH; and
each R⁵ is independently H, NH₂, OH, CO₂H, C(═O)NH₂, C(═O)NHOH, C(═O)NHNH₂, NHC(═NH)NH₂, NHC(═O)NH₂, NHC(═S)NH₂, PO₃H₂, SO₃H, or S(O)₂NH₂.

9. The composition of claim 8 wherein
A¹, A², A³, A⁴, and A⁵ are each independently CR¹;
each R¹ is independently H, C(O)R², C(O)OR², NHC(═O)NHR², NHC(═S)NHR², OC(═O)R², OC(═O)OR², S(O)₂OR², C(O)NR³R⁴, C(O)NR³OR⁴, C(O)NR²NR³R⁴, NR³R⁴, NR³C(O)R⁴, PO(OR³)(OR⁴), S(O)₂NR³R⁴, S(O)₂NR²NR³R⁴, S(O)₂NR³OR⁴, C₁-C₁₀ alkyl substituted with 0-3 R⁵, C₃-C₁₀ cycloalkyl substituted with 0-3 R⁵, C₂-C₁₀ alkenyl substituted with 0-3 R⁵, or aryl substituted with 0-5 R⁵;
R², R³, and R⁴ are each independently H, C₁-C₆ alkyl, C₃-C₆ cycloalkyl, benzyl, or phenyl; or R³ and R⁴ together form C₃-C₁₀ cycloalkyl optionally interrupted with O, S, NH, S(═O), S(O)₂, P(═O)(OH), C(═O)NH, NHC(═O), NHC(═O)NH, or NHC(═S)NH; and
each R⁵ is independently H, NH₂, OH, CO₂H, C(═O)NH₂, PO₃H₂, SO₃H, or S(O)₂NH₂.

10. The composition of claim 9 wherein
each R¹ is independently C(O)H, C(O)NH₂, C(O)NHNH₂, CO₂H, NHC(═O)NH₂, NHC(═S)NH₂, PO₃H₂, SO₃H, or S(O)₂NH₂.

11. The composition of claim 10 wherein the compound of formula 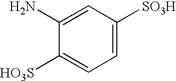
(I) is a compound of the formula:
or a pharmaceutically acceptable salt thereof.

12. The composition of claim 1 wherein the compound of formula (I) is present at a concentration of about 0.1 mg/mL to about 20 mg/mL.

13. The composition of claim 12 wherein the radioisotope is present at a level of about 20 mCi to about 2000 mCi and is present at a concentration of greater than about 5 mCi/mL of the radiopharmaceutical composition.

14. The composition of claim 13 wherein the radioisotope is ⁹⁰Y or ¹⁷⁷Lu.

15. The composition of claim 1 wherein the biomolecule is a peptidomimetic.

16. The composition of claim 1 wherein the biomolecule is a non-peptide.

17. The composition of claim 1 further comprising an effective stabilizing amount of a second stabilizer selected from the group consisting of ascorbic acid, benzyl alcohol, gentisic acid, an ester of gentisic acid, gentisyl alcohol, an ester of gentisyl alcohol, p-aminobenzoic acid, cystamine, cystamine, 5-amino-2-hydroxybenzoic acid, nicotinic acid, nicotinamide, propylene glycol, dextran, inositol, a compound of formula (I): 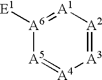
wherein,
E¹ is NH₂ or OH;
A¹, A², A³, A⁴ and A⁵ are each independently N, C(OH) or CR¹;
provided at least one of A¹, A², A³, A⁴ and A⁵ is not CH;
each R¹ is independently H, C(O)R², C(O)OR², NHC(═O)NHR², NHC(═S)NHR², OC(═O)R², OC(═O)OR², S(O)₂OR², C(O)NR³R⁴, C(O)NR³OR⁴, C(O)NR²NR³R⁴, NR³R⁴, NR³C(O)R⁴, PO(OR³)(OR⁴), S(O)₂NR³R⁴, S(O)₂NR²NR³R⁴, S(O)₂NR³OR⁴, C₁-C₁₀ alkyl substituted with 0-5 R⁵, C₃-C₁₀ cycloalkyl substituted with 0-5 R⁵, C₂-C₁o alkenyl substituted with 0-5 R⁵, or aryl substituted with 0-5 R⁵;
R², R³, and R⁴ are each independently H, C₁-C₆ alkyl, C₃-C₆ cycloalkyl, C₁-C₆ alkenyl, benzyl, or phenyl; or R³ and R⁴ together form C₃-C₁₀ cycloalkyl or C₃-C₁₀ cycloalkenyl, optionally interrupted with O, S, NH, S(═O), S(O)₂, P(═O)(OH), C(═O)NH, NHC(═O), NHC(═O)NH, or NHC(═S)NH; and
each R⁵ is independently H, NH₂, OH, CO₂H, C(═O)NH₂, C(═O)NHOH, C(═O)NHNH₂, NHC(═NH)NH₂, NHC(═O)NH₂, NHC(═S)NH₂, PO₃H₂, SO₃H, or S(O)₂NH₂;
or a pharmaceutically acceptable salt thereof.

18. The composition of claim 17 wherein the second stabilizer is present at a concentration of about 0.1 mg/mL to about 20 mg/mL.

19. A pharmaceutical composition comprising:
(1.) a radiolabeled pharmaceutical agent of the formula (II)
RI-Ch-Lₙ-(BM)ₓ (II); and
(2.) an effective stabilizing amount of a compound of formula (I): 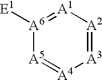
wherein
RI is ^{99m}Tc, ¹³¹I, ¹²⁵I, ¹²³I, ^{117m}Sn, ¹¹¹In, ⁹⁷Ru, ²⁰³Pb, ⁶⁷Ga, ⁶⁸Ga, ⁸⁹Zr, ⁹⁰Y, ¹⁷⁷Lu, ¹⁴⁹Pm, ¹⁵³Sm, ¹⁶⁶Ho, ¹³¹I, ³²P, ²¹¹At, ⁴⁷Sc, ¹⁰⁹Pd, ¹⁰⁵Rh, ¹⁸⁶Re, ¹⁸⁸Re, ⁶⁰Cu, ⁶²Cu, ⁶⁴Cu or ⁶⁷Cu;
Cₕ is a metal chelator or is a direct linkage;
Lₙ is a linking group or is a direct linkage;
each BM is independently an antibody, an antibody fragment, a peptide, a peptidomimetic, or a non-peptide;
x is 1 to about 10;
E¹ is NH₂ or OH;
A¹, A², A³, A⁴ and A⁵ are each independently N, C(OH) or CR¹;
provided at least one of A¹, A², A³, A⁴ and A⁵ is not CH;
each R¹ is independently H, C(O)R², C(O)OR², NHC(═O)NHR², NHC(═S)NHR², OC(═O)R², OC(═O)OR², S(O)₂OR², C(O)NR³R⁴, C(O)NR³OR⁴, C(O)NR²NR³R⁴, NR³R⁴, NR³C(O)R⁴, PO(OR³)(OR⁴) S(O)₂NR³R⁴, S(O)₂NR²NR³R⁴, S(O)₂NR³OR⁴, C₁-C₁₀ alkyl substituted with 0-5 R⁵, C₃-C₁₀ cycloalkyl substituted with 0-5 R⁵, C₂-C₁₀ alkenyl substituted with 0-5 R⁵, or aryl substituted with 0-5 R⁵;
R², R³, and R⁴ are each independently H, C₁-C₆ alkyl, C₃-C₆ cycloalkyl, C₁-C₆ alkenyl, benzyl, or phenyl; or R³ and R⁴ together form C₃-C₁₀ cycloalkyl or C₃-C₁₀ cycloalkenyl, optionally interrupted with O, S, NH, S(═O), S(O)₂, P(═O)(OH), C(═O)NH, NHC(═O), NHC(═O)NH, or NHC(═S)NH; and
each R⁵ is independently H, NH₂, OH, CO₂H, C(═O)NH₂, C(═O)NHOH, C(═O)NHNH₂, NHC(═NH)NH₂, NHC(═O)NH₂, NHC(═S)NH₂, PO₃H₂, SO₃H, or S(O)₂NH₂;
or a pharmaceutically acceptable salt thereof;
provided the compound of formula (I) is not (1) a substituted monohydroxyl aromatic compound; (2) a substituted dihydroxyl aromatic compound, in which the two hydroxyl groups are not adjacent to each other; (3) a substituted monohydroxyl-monoamino aromatic compound, in which the hydroxyl group and amino group are not adjacent to each other; or (4) an ortho, meta, or para aminobenzioc acid.

20. The composition of claim 19 wherein
E¹ is OH;
A¹, A², A³, and A⁴ are each independently C(OH) or CR¹;
A⁵ is C(OH);
each R¹ is independently H, C(O)R², C(O)OR², NHC(═O)NHR², NHC(═S)NHR², OC(═O)R², OC(═O)OR², S(O)₂OR², C(O)NR³R⁴, C(O)NR³OR⁴, C(O)NR²NR³R⁴, NR³R⁴, NR³C(O)R⁴, PO(OR³)(OR⁴), S(O)₂NR³R⁴, S(O)₂NR²NR³R⁴, S(O)₂NR³OR⁴, C₁-C₁₀ alkyl substituted with 0-3 R⁵, C₃-C₁₀ cycloalkyl substituted with 0-3 R⁵, C₂-C₁₀ alkenyl substituted with 0-3 R⁵, or aryl substituted with 0-5 R⁵;
R², R³, and R⁴ are each independently H, C₁-C₆ alkyl, C₃-C₆ cycloalkyl, benzyl, or phenyl; or R³ and R⁴ together form C₃-C₁₀ cycloalkyl optionally interrupted with O, S, NH, S(═O), S(O)₂, P(═O)(OH), C(═O)NH, NHC(═O), NHC(═O)NH, or NHC(═S)NH; and
each R⁵ is independently H, NH₂, OH, CO₂H, C(═O)NH₂, PO₃H₂, SO₃H, or S(O)₂NH₂.

21. The composition of claim 20 wherein,
A⁴ is C(OH); and
each R¹ is independently C(O)H, C(O)NH₂, C(O)NHNH₂, CO₂H, NHC(═O)NH₂, NHC(═S)NH₂, PO₃H₂, SO₃H, or S(O)₂NH₂.

22. The composition of claim 21 wherein the compound of formula (I) is: 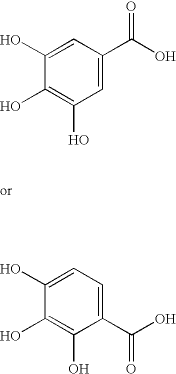
or a pharmaceutically acceptable salt thereof.

23. The composition of claim 19 wherein
E¹ is NH₂;
A¹, A², A³, and A⁴ are each independently C(OH) or CR¹;
A⁵ is C(OH);
each R¹ is independently H, C(O)R², C(O)OR², NHC(═O)NHR², NHC(═S)NHR², OC(═O)R², OC(═O)OR², S(O)₂OR², C(O)NR³R⁴, C(O)NR³OR⁴, C(O)NR²NR³R⁴, NR³R⁴, NR³C(O)R⁴ PO(OR³)(OR⁴), S(O)₂NR³R⁴, S(O)₂NR²NR³R⁴, S(O)₂NR³OR⁴, C₁-C₁₀ alkyl substituted with 0-3 R⁵, C₃-C₁₀ cycloalkyl substituted with 0-3 R⁵, C₂-C₁₀ alkenyl substituted with 0-3 R⁵, or aryl substituted with 0-5 R⁵;
R², R³, and R⁴ are each independently H, C₁-C₆ alkyl, C₃-C₆ cycloalkyl, benzyl, or phenyl; or R³ and R⁴ together form C₃-C₁₀ cycloalkyl optionally interrupted with O, S, NH, S(═O), S(O)₂, P(═O)(OH), C(═O)NH, NHC(═O), NHC(═O)NH, or NHC(═S)NH; and
each R⁵ is independently H, NH₂, OH, CO₂H, C(═O)NH₂, PO₃H₂, SO₃H, or S(O)₂NH₂.

24. The composition of claim 23 wherein
each R¹ is independently C(O)H, C(O)NH₂, C(O)NHNH₂, CO₂H, NHC(═O)NH₂, NHC(═S)NH₂, PO₃H₂, SO₃H, or S(O)₂NH₂.

25. The composition of claim 24 wherein compound of the formula (I) is a compound of the formula: 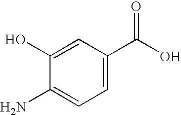
or a pharmaceutically acceptable salt thereof.

26. The composition of claim 19 wherein
A¹, A², A³, A⁴, and A⁵ are each independently N, C(OH) or CR¹; provided that A⁵ is not C(OH);
each R¹ is independently H, C(O)R², C(O)OR², NHC(═O)NHR², NHC(═S)NHR², OC(═O)R², OC(═O)OR², S(O)₂OR², C(O)NR³R⁴, C(O)NR³OR⁴, C(O)NR²NR³R⁴, NR³R⁴, NR³C(O)R⁴, PO(OR³)(OR⁴), S(O)₂NR³R⁴, S(O)₂NR²NR³R⁴, S(O)₂NR³OR⁴, C₁-C₁₀ alkyl substituted with 0-5 R⁵, C₃-C₁₀ cycloalkyl substituted with 0-5 R⁵, C₂-C₁₀ alkenyl substituted with 0-5 R⁵, or aryl substituted with 0-5 R⁵;
R², R³, and R⁴ are each independently H, C₁-C₆ alkyl, C₃-C₆ cycloalkyl, C₁-C₆ alkenyl, benzyl, or phenyl; or R³ and R⁴ together form C₃-C₁₀ cycloalkyl or C₃-C₁₀ cycloalkenyl optionally interrupted with O, S, NH, S(═O), S(O)₂, P(═O)(OH), C(═O)NH, NHC(═O), NHC(═O)NH, or NHC(═S)NH; and
each R⁵ is independently H, NH₂, OH, CO₂H, C(═O)NH₂, C(═O)NHOH, C(═O)NHNH₂, NHC(═NH)NH₂, NHC(═O)NH₂, NHC(═S)NH₂, PO₃H₂, SO₃H, or S(O)₂NH₂.

27. The composition of claim 26 wherein
A¹, A², A³, A⁴, and A⁵ are each independently CR¹;
each R¹ is independently H, C(O)R², C(O)OR², NHC(═O)NHR, NHC(═S)NHR², OC(═O)R², OC(═O)OR², S(O)₂OR², C(O)NR³R⁴, C(O)NR³OR⁴, C(O)NR²NR³R⁴, NR³R⁴, NR³C(O)R⁴, PO(OR³)(OR⁴), S(O)₂NR³R⁴, S(O)₂NR²NR³R⁴, S(O)₂NR³OR⁴, C₁-C₁₀ alkyl substituted with 0-3 R⁵, C₃-C₁₀ cycloalkyl substituted with 0-3 R⁵, C₂-C₁₀ alkenyl substituted with 0-3 R⁵, or aryl substituted with 0-5 R⁵;
R², R³, and R⁴ are each independently H, C₁-C₆ alkyl, C₃-C₆ cycloalkyl, benzyl, or phenyl; or R³ and R⁴ together form C₃-C₁₀ cycloalkyl optionally interrupted with O, S, NH, S(═O), S(O)₂, P(═O)(OH), C(═O)NH, NHC(═O), NHC(═O)NH, or NHC(═S)NH; and
each R⁵ is independently H, NH₂, OH, CO₂H, C(═O)NH₂, PO₃H₂, SO₃H, or S(O)₂NH₂.

28. The composition of claim 27 wherein
each R¹ is independently C(O)H, C(O)NH₂, C(O)NHNH₂, CO₂H, NHC(═O)NH₂, NHC(═S)NH₂, PO₃H₂, SO₃H, or S(O)₂NH₂.

29. The composition of claim 28 wherein the compound of formula 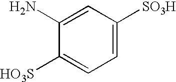
(I) is a compound of the formula:
or a pharmaceutically acceptable salt thereof.

30. The composition of claim 19 wherein the compound of formula (I) is present at a concentration of about 0.1 mg/mL to about 20 mg/mL.

31. The composition of claim 30 wherein the radioisotope is present at a level of about 20 mCi to about 2000 mCi and at a concentration of greater than about 5 mCi/mL of the radiopharmaceutical composition.

32. The composition of claim 31 wherein the radioisotope is ⁹⁰Y or ¹⁷⁷Lu.

33. The composition of claim 19 wherein the biomolecule is a peptide.

34. The composition of claim 19 wherein the biomolecule is a non-peptide.

35. The composition of claim 19 wherein the biomolecule is a peptidomimetic.

36. The composition of claim 19 wherein the biomolecule is an antibody.

37. The composition of claim 19 wherein the biomolecule is an antibody fragment.

38. The composition of claim 19 further comprising an effective stabilizing amount of a second stabilizer selected from the group consisting of ascorbic acid, benzyl alcohol, gentisic acid, an ester of gentisic acid, gentisyl alcohol, an ester of gentisyl alcohol, p-aminobenzoic acid, cystamine, cystamine, 5-amino-2-hydroxybenzoic acid, nicotinic acid, nicotinamide, propylene glycol, dextran, inositol, a compound of formula (I): 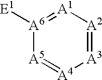
wherein,
E¹ is NH₂ or OH;
A¹, A², A³, A⁴ and A⁵ are each independently N, C(OH) or CR¹;
provided at least one of A¹, A², A³, A⁴ and A⁵ is not CH;
each R¹ is independently H, C(O)R², C(O)OR², NHC(═O)NHR², NHC(═S)NHR², OC(═O)R², OC(═O)OR², S(O)₂OR², C(O)NR³R⁴, C(O)NR³OR⁴, C(O)NR²NR³R⁴, NR³R⁴, NR³C(O)R⁴, PO(OR³)(OR⁴), S(O)₂NR³R⁴, S(O)₂NR²NR³R⁴, S(O)₂NR³OR⁴, C₁-C₁₀ alkyl substituted with 0-5 R⁵, C₃-C₁₀ cycloalkyl substituted with 0-5 R⁵, C₂-C₁₀ alkenyl substituted with 0-5 R⁵, or aryl substituted with 0-5 R⁵;
R², R³, and R⁴ are each independently H, C₁-C₆ alkyl, C₃-C₆ cycloalkyl, C₁-C₆ alkenyl, benzyl, or phenyl; or R³ and R⁴ together form C₃-C₁₀ cycloalkyl or C₃-C₁₀ cycloalkenyl, optionally interrupted with O, S, NH, S(═O), S(O)₂, P(═O)(OH), C(═O)NH, NHC(═O), NHC(═O)NH, or NHC(═S)NH; and
each R⁵ is independently H, NH₂, OH, CO₂H, C(═O)NH₂, C(═O)NHOH, C(═O)NHNH₂, NHC(═NH)NH₂, NHC(═O)NH₂, NHC(═S)NH₂, PO₃H₂, SO₃H, or S(O)₂NH₂;
or a pharmaceutically acceptable salt thereof.

39. The composition of claim 38 wherein the second stabilizer is present at a concentration of about 0.1 mg/mL to about 20 mg/mL.

40. A method for preparing a stable radiopharmaceutical composition of claim 1 comprising:
combining in the absence of oxygen, the radiolabeled pharmaceutical agent of the formula (II):
RI-Ch-Lₙ-(BM)ₓ (II); and
an effective stabilizing amount of the stabilizer of the formula (I).

41. The method of claim 40 wherein the radiolabeled pharmaceutical agent and the stabilizer are combined in a container.

42. The method of claim 41 wherein an oxygen free head-space is maintained in the container.

43. The method of claim 40 further comprising cooling to a temperature of less than about −20° C.

44. The method of claim 40 further comprising storing to a temperature of less than about −20° C.

45. A method for preparing a stable radiopharmaceutical composition of claim 1 comprising:
combining in a container, in the absence of oxygen, the radiolabeled pharmaceutical agent of the formula RI-Ch-Lₙ-(BM)ₓ and an effective stabilizing amount of the stabilizer of the formula (I);
maintaining an oxygen free head-space in the container;
cooling the container to a temperature of less than about −20° C.; and
storing the container to a temperature of less than about −20° C.

46. A method for treating or preventing thromboembolic disorders, atherosclerosis, infection, inflammation, transplant rejection, cancer or a disease state that is associated with the following receptors: a cyclic IIb/IIIa receptor, a fibrinogen receptor, a myocardial receptor, a renal receptor, LTβ4, selectin, growth factor (PDGF, VEGF, EGF, FGF, TNF MCSF or an interleukin Il1-8), a receptor that is expressed or upregulated in angiogenic tumor vasculature, αvβ3, αvβ5, α5β1, α4β1, α1β1, or α2β2, α5β1, αvβ3, α5β1, or tyrosine kinases (e.g., epidermal growth factor receptor (EGFR) family in a mammalian tissue inflicted with or at risk thereof comprising contacting the mammalian tissue with an effective amount of a composition of claim 1.

47. The method of claim 46 wherein the mammal is a human.

48. The method of claim 46 wherein the contacting is in vivo.

49. The method of claim 46 wherein the contacting is in vitro.

50. A method for treating or prev enting cancer,thr omboembolic disorders, atherosclerosis, infection, inflammation, transplant rejection, cancer or a disease state that is associated with the following receptors: a cyclic IIb/IIIa receptor, a fibrinogen receptor, a myocardial receptor, a renal receptor, LTβ4, selectin, growth factor (PDGF, VEGF, EGF, FGF, TNF MCSF or an interleukin Il1-8), a receptor that is expressed or upregulated in angiogenic tumor vasculature, αvβ3, αvβ5, α5β1, α4β1, α1β1, or α2β2, α5β1, αvβ3, α5β1, or tyrosine kinases (e.g., epidermal growth factor receptor (EGFR) family in a patient (e.g., mammal) inflicted with or at risk thereof comprising administering to the mammal in need of such treatment or prevention an effective amount of a composition of claim 1.

51. The method of claim 50 wherein the mammal is a human.

52. A method for imaging a tumor on or in a mammalian tissue inflicted with a tumor comprising contacting the mammalian tissue with an effective amount of a composition of claim 1; and detecting the presence of the radiolabeled pharmaceutical; wherein the ligand has an affinity for tumor cells.

53. The method of claim 52 wherein the mammal is a human.

54. The method of claim 52 wherein the contacting is in vivo.

55. The method of claim 52 wherein the contacting is in vitro.

56. A method for imaging a tumor in a mammal inflicted with a tumor comprising administering to the mammal an effective amount of a composition of claim 1; and detecting the presence of the radiolabeled pharmaceutical.

57. The method of claim 56 wherein the mammal is a human.

58. The method of claim 56 wherein the tumor is located in the breast, lung, thyroid, lymph node, kidney, ureter, bladder, ovary, teste, prostate, bone, skeletal muscle, bone marrow, stomach, esophagus, small bowel, colon, rectum, pancreas, liver, smooth muscle, brain, spinal cord, nerves, ear, eye, nasopharynx, oropharynx, salivary gland, or the heart.

59. A pharmaceutical composition of claim 1 for use in medical therapy or diagnosis.

60. The use of a pharmaceutical composition of claim 1 for the manufacture of a medicament for imaging or treating a tumor in a mammal.

61. The use of a pharmaceutical composition of claim 1 for the manufacture of a medicament for treating a tumor, a thromboembolic disorder, atherosclerosis, an infection, inflammation, transplant rejection or a disease state that is associated with the following receptors: a cyclic IIb/IIIa receptor, a fibrinogen receptor, a myocardial receptor, a renal receptor, LTβ4, selectin, growth factor (PDGF, VEGF, EGF, FGF, TNF MCSF or an interleukin Il1-8), a receptor that is expressed or upregulated in angiogenic tumor vasculature, αvβ3, αvβ5, α5β1, α4β1, α1β1, or α2β2, α5β1, αvβ3, α5β1, or tyrosine kinases (e.g., epidermal growth factor receptor (EGFR) family in a mammal.

62. A diagnostic composition comprising an effective diagnostic amount of a radiolabeled agent RI-Ch-Ln-(BM)ₓ, an effective stabilizing amount of a compound of formula (I) of claim 1, and a physiologically acceptable carrier or excipient.

63. A compound of formula (I) of claim 1 for use in preparing a stable radio-imaging composition comprising an effective diagnostic amount of a radiolabeled agent RI-Ch-Ln-(BM)ₓ, an effective stabilizing amount of a compound of formula (I) of claim 1, and a physiologically acceptable carrier or excipient.

64. A scintigraphic diagnostic composition comprising an effective stabilizing amount of a compound of formula (I)and a radiolabeled agent RI-Ch-Ln-(BM)ₓ of claim 1.

65. A method of in vivo radio-imaging comprising:
(a) introducing a radioisotope (RI) to a solution comprising a compound Ch-Ln-(BM)ₓ and an effective stabilizing amount of a compound of formula (I) of claim 1, to form a labeled solution;
(b) administering the labeled solution in vivo; and
(c) detecting localization of the radioisotope in vivo.

66. A method of in vitro radio-imaging a targeted receptor of a tissue comprising:
(a) administering an effective diagnostic amount of a composition according to claim 62 to the tissue; and
(b) detecting localization of the radiolabeled agent at the targeted receptor.

67. The method according to claim 66 wherein the targeted receptor is selected from the group consisting of a cyclic IIb/IIIa receptor, a fibrinogen receptor, a myocardial receptor, a renal receptor, LTβ4, selectin, growth factor (PDGF, VEGF, EGF, FGF, TNF MCSF or an interleukin Il1-8), a receptor that is expressed or upregulated in angiogenic tumor vasculature, αvβ3, αvβ5, α5β1, α4β1, α1β1, or α2β2, α5β1, αvβ3, α5β1, and tyrosine kinases (e.g., epidermal growth factor receptor (EGFR) family.

68. A method of radio-imaging a targeted site within a patient's body comprising:
(a) administering an effective diagnostic amount of a composition according to claim 62 to the patient; and
(b) detecting localization of the radiolabeled agent at the targeted site.

69. A method of radio-imaging for prostate cancer or other tissues having an androgen receptor in a patient comprising:
(a) administering an effective diagnostic amount of a composition according to claim 62; and
(b) detecting the presence of the radiolabeled agent RI-Ch-Ln-(BM)ₓ bound to the androgen receptor.

70. A method of radio-imaging metastasized cancer cells comprising contacting an effective diagnostic amount of a radiolabeled agent RI-Ch-Ln-(BM)ₓ and an effective stabilizing amount of a compound of formula (I) of claim 1, with a composition comprising ST receptor wherein said radiolabeled agent is capable of targeting a ST receptor.

71. A method of radio-imaging a patient's organ comprising:
(a) administering an effective diagnostic amount of a radiolabeled agent RI-Ch-Ln-(BM)ₓ, and an effective stabilizing amount of a compound of formula (I) of claim 1 to a patient in need of such radioimaging; and
(b) and detecting the presence of the radiolabeled agent bound to said organ.

72. The method of claim 71 wherein the organ is selected from the group consisting of the breast, lung, thyroid, lymph node, kidney, ureter, bladder, ovary, teste, prostate, bone, skeletal muscle, bone marrow, stomach, esophagus, small bowel, colon, rectum, pancreas, liver, smooth muscle, brain, spinal cord, nerves, ear, eye, nasopharynx, oropharynx, salivary gland, and the heart.

73. A method of delivering a radionuclide to a target location, comprising:
providing a radiolabeled agent RI-Ch-Ln-(BM)ₓ and providing an effective stabilizing amount of a compound of formula (I) of claim 1.

74. The method of claim 73 wherein the target location is a cancer cell.

75. A kit for preparing a radio-imaging composition, the kit comprising a sealed vial containing a predetermined quantity of a radiolabeled agent RI-Ch-Ln-(BM)ₓ and an effective stabilizing amount of a compound of formula (I) of claim 1.

76. A kit comprising a plurality-vial system of a radio-imaging composition of claim 62 and a diluent, comprising:
(a) a first vial comprising a predetermined quantity of a radiolabelled agent RI-Ch-Ln-(BM)ₓ and an effective stabilizing amount of a compound of formula (I); and
(b) a second vial comprising a pharmaceutically acceptable carrier or diluent.

77. A pharmaceutical composition comprising a radiolabeled agent RI-Ch-Ln-(BM)ₓ, an effective stabilizing amount of a compound of formula (I) of claim 1, and optionally an effective stabilizing amount of a second stabilizer compound selected from the group consisting of of ascorbic acid, benzyl alcohol, gentisic acid, an ester of gentisic acid, gentisyl alcohol, an ester of gentisyl alcohol, p-aminobenzoic acid, cystamine, cystamine, 5-amino-2-hydroxybenzoic acid, nicotinic acid, nicotinamide, propylene glycol, dextran, inositol, a compound of formula (I), or a pharmaceutically acceptable salt thereof, and a pharmaceutically acceptable carrier.

78. A method of preparing a stable radiopharmaceutical composition, comprising:
providing a radiolabeled agent RI-Ch-Ln-(BM)ₓ and providing an effective stabilizing amount of a compound of formula (I) of claim 1.

79. A method of treating cancer, comprising administering to a patient, in need thereof, a therapeutically effective amount of a pharmaceutical composition according to claim 77 and optionally at least one agent selected from the group consisting of a chemotherapeutic agent and a radiosensitizer agent, or a pharmaceutically acceptable salt thereof.

80. The method according to claim 79 wherein administering is concurrent.

81. The method according to claim 79 wherein administering is sequential.

82. The method of treating cancer according to claim 79 wherein the cancer is a vascularized tumor (i.e. a solid tumor).

83. The method according to claim 79 wherein the cancer is selected from the group consisting of carcinomas of the lung, breast, ovary, stomach, pancreas, larynx, esophagus, testes, liver, parotid, biliary tract, colon, rectum, cervix, uterus, endometrium, kidney, bladder, prostate, thyroid, squamous cell carcinomas, adenocarcinomas, small cell carcinomas, melanomas, gliomas, and neuroblastomas.

84. The method according to claim 79 wherein the chemotherapeutic agent is selected from the group consisting of mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma, colony stimulating factor-1, colony stimulating factor-2, denileukin diftitox, interleukin-2, and leutinizing hormone releasing factor.

85. The method according to claim 79 wherein the radiosensitizer agent is selected from the group consiting of 2-(3-nitro-1,2,4-triazol-1-yl)-N-(2-methoxyethyl)acetamide, N-(3-nitro-4-quinolinyl)-4-morpholinecarboxamidine, 3-amino-1,2,4-benzotriazine-1,4-dioxide, N-(2-hydroxyethyl)-2-nitroimidazole-1-acetamide, 1-(2-nitroimidazol-1-yl)-3-(1-piperidinyl)-2-propanol, and 1-(2-nitro-1-imidazolyl)-3-(1-aziridino)-2-propanol.

86. A kit for treating cancer, comprising a therapeutically effective amount of a pharmaceutical composition according to claim 77 and optionally at least one agent selected from the group consisting of a chemotherapeutic agent and a radiosensitizer agent,or a pharmaceutically acceptable salt thereof.

87. The kit according to claim 86 wherein said kit comprises a plurality of separate containers, wherein at least one of said containers contains a therapeutically effective amount of a pharmaceutical composition according to claim 77, and at least another of said containers contains one or more agents selected from the group consisting of a chemotherapeutic agent and a radiosensitizer agent, or a pharmaceutically acceptable salt thereof, and a pharmaceutically acceptable carrier.

88. The kit according to claim 86, wherein the chemotherapeutic agent is selected from the group consisting of mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma, colony stimulating factor-1, colony stimulating factor-2, denileukin diftitox, interleukin-2, and leutinizing hormone releasing factor.

89. The kit according to claim 86, wherein the chemotherapeutic agent is selected from the group consisting of mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, and lisuride.

90. The kit according to claim 86 wherein the chemotherapeutic agent is selected from the group consisting of oxymetholone, tamoxifen, progesterone, mepitiostane, epitiostanol, and formestane.

91. The kit according to claim 86 wherein the chemotherapeutic agent is selected from the group consisting of interferon-alpha, interferon-2 alpha, interferon-beta, interferon-gamma, colony stimulating factor-1, colony stimulating factor-2, denileukin diftitox, interleukin-2, and leutinizing hormone releasing factor.

92. The kit according to claim 86, wherein the radiosensitizer agent is selected from the group consiting of 2-(3-nitro-1,2,4-triazol-1-yl)-N-(2-methoxyethyl)acetamide, N-(3-nitro-4-quinolinyl)-4-morpholinecarboxamidine, 3-amino-1,2,4-benzotriazine-1,4-dioxide, N-(2-hydroxyethyl)-2-nitroimidazole-1-acetamide, 1-(2-nitroimidazol-1-yl)-3-(1-piperidinyl)-2-propanol, and 1-(2-nitro-1-imidazolyl)-3-(1-aziridino)-2-propanol.
